(19) United States
(12) Patent Application Publication
(10) Pub. No.: US 2025/0184679 A1
(43) Pub. Date: Jun. 5, 2025

(54) HEARING HEALTH USER INTERFACES

(71) Applicant: Apple Inc., Cupertino, CA (US)

(72) Inventors: Christopher D. LAURITZEN, San Francisco, CA (US); Nicholas D. FELTON, Sunnyvale, CA (US); Ruchi N. GOSWAMI, Sunnyvale, CA (US); Christopher J. ROMNEY, San Jose, CA (US)

(21) Appl. No.: 18/963,199

(22) Filed: Nov. 27, 2024

Related U.S. Application Data
(60) Provisional application No. 63/724,238, filed on Nov. 22, 2024.
(60) Provisional application No. 63/691,213, filed on Sep. 5, 2024.
(60) Provisional application No. 63/606,046, filed on Dec. 4, 2023.

(51) Int. Cl. H04R 25/00; H04R 29/00

(52) U.S. Cl. CPC H04R 25/70; H04R 25/554; H04R 25/558; H04R 29/001; H04R 29/008; H04R 2225/41; H04R 2225/55

(57) ABSTRACT

The present disclosure generally relates to user interfaces for hearing health. In some embodiments, a computer system prompts a user to initiate a hearing test. In some embodiments, different types of audio are modified using different audio characteristics. In some embodiments, a visual indication of a current level of environmental noise is displayed. In some embodiments, the computer system detects a user input indicating the faintest volume at which a user can hear test audio.

### CROSS-REFERENCE TO RELATED APPLICATIONS

This application relates to U.S. Provisional Patent Application No. 63/724,238, entitled “HEARING HEALTH USER INTERFACES” and filed on Nov. 22, 2024, and U.S. Provisional Patent Application No. 63/691,213, entitled “HEARING HEALTH USER INTERFACES” and filed on Sep. 5, 2024 and U.S. Provisional Patent Application No. 63/606,046, entitled “HEARING HEALTH USER INTERFACES” and filed on Dec. 4, 2023, the contents of each of which are hereby incorporated by reference in their entireties.

### FIELD

The present disclosure relates generally to computer user interfaces, and more specifically to techniques for testing and/or adjusting for hearing loss.

### BACKGROUND

Some individuals experience varying levels of hearing loss in one or both ears. Individuals can participate in a hearing test, which gauges how well the user can hear. Many hearing tests involve wearing earphones that play short tones at different volumes and at different pitches, usually into one ear at a time. The tones are used to test whether the individual can hear high-pitched or low-pitched sounds and quiet or loud sounds. The results of the test indicate whether there is hearing loss and the hearing loss is often categorized, such as by mild, moderate, severe, and profound hearing loss.

### BRIEF SUMMARY

Some techniques for managing hearing health using electronic devices, however, are generally cumbersome and inefficient. For example, some existing techniques use a complex and time-consuming user interface, which may include multiple key presses or keystrokes. Existing techniques require more time than necessary, wasting user time and device energy. This latter consideration is particularly important in battery-operated devices.

Accordingly, the present technique provides electronic devices with faster, more efficient methods and interfaces for managing hearing health. Such methods and interfaces optionally complement or replace other methods for managing hearing health. Such methods and interfaces reduce the cognitive burden on a user and produce a more efficient human-machine interface. For battery-operated computing devices, such methods and interfaces conserve power and increase the time between battery charges.

In some embodiments, a method is described. The method comprises: at a computer system that is in communication with one or more input devices: while not paired with a first audio device, detecting, via the one or more input devices, a request to pair the first audio device with the computer system; and in response to detecting the request to pair the first audio device with the computer system, initiating a process to pair the first audio device with the computer system, wherein the process to pair the first audio device with the computer system includes: pairing the first audio device with the computer system; in accordance with a determination that the first audio device is a first type of audio device, providing an option to perform a hearing test that uses the first audio device; and in accordance with a determination that the first audio device is a second type of audio device that is different from the first type of audio device, forgoing providing the option to perform the hearing test that uses the first audio device.

In some embodiments, a non-transitory computer-readable storage medium is described. The non-transitory computer-readable storage medium stores one or more programs configured to be executed by one or more processors of a computer system that is in communication with one or more input devices, the one or more programs including instructions for: while not paired with a first audio device, detecting, via the one or more input devices, a request to pair the first audio device with the computer system; and in response to detecting the request to pair the first audio device with the computer system, initiating a process to pair the first audio device with the computer system, wherein the process to pair the first audio device with the computer system includes: pairing the first audio device with the computer system; in accordance with a determination that the first audio device is a first type of audio device, providing an option to perform a hearing test that uses the first audio device; and in accordance with a determination that the first audio device is a second type of audio device that is different from the first type of audio device, forgoing providing the option to perform the hearing test that uses the first audio device.

In some embodiments, a transitory computer-readable storage medium is described. The transitory computer-readable storage medium stores one or more programs configured to be executed by one or more processors of a computer system that is in communication with one or more input devices, the one or more programs including instructions for: while not paired with a first audio device, detecting, via the one or more input devices, a request to pair the first audio device with the computer system; and in response to detecting the request to pair the first audio device with the computer system, initiating a process to pair the first audio device with the computer system, wherein the process to pair the first audio device with the computer system includes: pairing the first audio device with the computer system; in accordance with a determination that the first audio device is a first type of audio device, providing an option to perform a hearing test that uses the first audio device; and in accordance with a determination that the first audio device is a second type of audio device that is different from the first type of audio device, forgoing providing the option to perform the hearing test that uses the first audio device.

In some embodiments, a computer system is described. The computer system is configured to communicate with one or more input devices. The computer system comprises: one or more processors; and memory storing one or more programs configured to be executed by the one or more processors, the one or more programs including instructions for: while not paired with a first audio device, detecting, via the one or more input devices, a request to pair the first audio device with the computer system; and in response to detecting the request to pair the first audio device with the computer system, initiating a process to pair the first audio device with the computer system, wherein the process to pair the first audio device with the computer system includes: pairing the first audio device with the computer system; in accordance with a determination that the first audio device is a first type of audio device, providing an option to perform a hearing test that uses the first audio device; and in accordance with a determination that the first audio device is a second type of audio device that is different from the first type of audio device, forgoing providing the option to perform the hearing test that uses the first audio device.

In some embodiments, a computer system is described. The computer system is configured to communicate with one or more input devices. The computer system comprises: means, while not paired with a first audio device, for detecting, via the one or more input devices, a request to pair the first audio device with the computer system; and means, responsive to detecting the request to pair the first audio device with the computer system, for initiating a process to pair the first audio device with the computer system, wherein the process to pair the first audio device with the computer system includes: pairing the first audio device with the computer system; in accordance with a determination that the first audio device is a first type of audio device, providing an option to perform a hearing test that uses the first audio device; and in accordance with a determination that the first audio device is a second type of audio device that is different from the first type of audio device, forgoing providing the option to perform the hearing test that uses the first audio device.

In some embodiments, a computer program product is described. The computer program product comprises one or more programs configured to be executed by one or more processors of a computer system that is in communication with one or more input devices, the one or more programs including instructions for: while not paired with a first audio device, detecting, via the one or more input devices, a request to pair the first audio device with the computer system; and in response to detecting the request to pair the first audio device with the computer system, initiating a process to pair the first audio device with the computer system, wherein the process to pair the first audio device with the computer system includes: pairing the first audio device with the computer system; in accordance with a determination that the first audio device is a first type of audio device, providing an option to perform a hearing test that uses the first audio device; and in accordance with a determination that the first audio device is a second type of audio device that is different from the first type of audio device, forgoing providing the option to perform the hearing test that uses the first audio device.

In some embodiments, a method is performed. The method comprises: at a computer system that includes a first audio device with one or more speakers: accessing a first set of audio characteristics that is based on a hearing test; accessing a second set of audio characteristics that is different from the first set of audio characteristics and is based on the hearing test; receiving first audio; and in response to receiving the first audio: in accordance with a determination that the first audio is a first type of audio, outputting, via the one or more speakers of the first audio device, first modified audio of the first audio that has been modified based on the first set of audio characteristics; and in accordance with a determination that the first audio is a second type of audio that is different from the first type of audio, outputting, via the one or more speakers of the first audio device, second modified audio of the first audio that has been modified based on the second set of audio characteristics.

In some embodiments, a non-transitory computer-readable storage medium is described. The non-transitory computer-readable storage medium stores one or more programs configured to be executed by one or more processors of a computer system that includes a first audio device with one or more speakers, the one or more programs including instructions for: accessing a first set of audio characteristics that is based on a hearing test; accessing a second set of audio characteristics that is different from the first set of audio characteristics and is based on the hearing test; receiving first audio; and in response to receiving the first audio: in accordance with a determination that the first audio is a first type of audio, outputting, via the one or more speakers of the first audio device, first modified audio of the first audio that has been modified based on the first set of audio characteristics; and in accordance with a determination that the first audio is a second type of audio that is different from the first type of audio, outputting, via the one or more speakers of the first audio device, second modified audio of the first audio that has been modified based on the second set of audio characteristics.

In some embodiments, a transitory computer-readable storage medium is described. The transitory computer-readable storage medium stores one or more programs configured to be executed by one or more processors of a computer system that includes a first audio device with one or more speakers, the one or more programs including instructions for: accessing a first set of audio characteristics that is based on a hearing test; accessing a second set of audio characteristics that is different from the first set of audio characteristics and is based on the hearing test; receiving first audio; and in response to receiving the first audio: in accordance with a determination that the first audio is a first type of audio, outputting, via the one or more speakers of the first audio device, first modified audio of the first audio that has been modified based on the first set of audio characteristics; and in accordance with a determination that the first audio is a second type of audio that is different from the first type of audio, outputting, via the one or more speakers of the first audio device, second modified audio of the first audio that has been modified based on the second set of audio characteristics.

In some embodiments, a computer system is described. The computer system includes a first audio device with one or more speakers, and comprises: one or more processors; and memory storing one or more programs configured to be executed by the one or more processors, the one or more programs including instructions for: accessing a first set of audio characteristics that is based on a hearing test; accessing a second set of audio characteristics that is different from the first set of audio characteristics and is based on the hearing test; receiving first audio; and in response to receiving the first audio: in accordance with a determination that the first audio is a first type of audio, outputting, via the one or more speakers of the first audio device, first modified audio of the first audio that has been modified based on the first set of audio characteristics; and in accordance with a determination that the first audio is a second type of audio that is different from the first type of audio, outputting, via the one or more speakers of the first audio device, second modified audio of the first audio that has been modified based on the second set of audio characteristics.

In some embodiments, a computer system is described. The computer system includes a first audio device with one or more speakers, and comprises: means for accessing a first set of audio characteristics that is based on a hearing test; means for accessing a second set of audio characteristics that is different from the first set of audio characteristics and is based on the hearing test; means for receiving first audio; and means, responsive to receiving the first audio, for: in accordance with a determination that the first audio is a first type of audio, outputting, via the one or more speakers of the first audio device, first modified audio of the first audio that has been modified based on the first set of audio characteristics; and in accordance with a determination that the first audio is a second type of audio that is different from the first type of audio, outputting, via the one or more speakers of the first audio device, second modified audio of the first audio that has been modified based on the second set of audio characteristics.

In some embodiments, a computer program product is described. The computer program product comprises one or more programs configured to be executed by one or more processors of a computer system that includes a first audio device with one or more speakers, the one or more programs including instructions for: accessing a first set of audio characteristics that is based on a hearing test; accessing a second set of audio characteristics that is different from the first set of audio characteristics and is based on the hearing test; receiving first audio; and in response to receiving the first audio: in accordance with a determination that the first audio is a first type of audio, outputting, via the one or more speakers of the first audio device, first modified audio of the first audio that has been modified based on the first set of audio characteristics; and in accordance with a determination that the first audio is a second type of audio that is different from the first type of audio, outputting, via the one or more speakers of the first audio device, second modified audio of the first audio that has been modified based on the second set of audio characteristics.

In some embodiments, a method is described. The method comprises: at a computer system that is in communication with a display generation component and one or more audio devices: during a process to perform a hearing test: displaying, via the display generation component, a visual indication of a current level of environmental noise, wherein the visual indication visually changes as the current level of environmental noise changes; while displaying the visual indication of the current level of environmental noise, detecting, via the one or more audio devices, a change in the current level of environmental noise; and in response to detecting the change in the current level of environmental noise, updating display, via the display generation component, of the visual indication of the current level of environmental noise to correspond to the current level of environmental noise.

In some embodiments, a non-transitory computer-readable storage medium is described. The non-transitory computer-readable storage medium stores one or more programs configured to be executed by one or more processors of a computer system that is in communication with a display generation component and one or more audio devices, the one or more programs including instructions for: during a process to perform a hearing test: displaying, via the display generation component, a visual indication of a current level of environmental noise, wherein the visual indication visually changes as the current level of environmental noise changes; while displaying the visual indication of the current level of environmental noise, detecting, via the one or more audio devices, a change in the current level of environmental noise; and in response to detecting the change in the current level of environmental noise, updating display, via the display generation component, of the visual indication of the current level of environmental noise to correspond to the current level of environmental noise.

In some embodiments, a transitory computer-readable storage medium is described. The transitory computer-readable storage medium stores one or more programs configured to be executed by one or more processors of a computer system that is in communication with a display generation component and one or more audio devices, the one or more programs including instructions for: during a process to perform a hearing test: displaying, via the display generation component, a visual indication of a current level of environmental noise, wherein the visual indication visually changes as the current level of environmental noise changes; while displaying the visual indication of the current level of environmental noise, detecting, via the one or more audio devices, a change in the current level of environmental noise; and in response to detecting the change in the current level of environmental noise, updating display, via the display generation component, of the visual indication of the current level of environmental noise to correspond to the current level of environmental noise.

In some embodiments, a computer system is described. The computer system is configured to communicate with a display generation component and one or more audio devices, comprising: one or more processors; and memory storing one or more programs configured to be executed by the one or more processors, the one or more programs including instructions for: during a process to perform a hearing test: displaying, via the display generation component, a visual indication of a current level of environmental noise, wherein the visual indication visually changes as the current level of environmental noise changes; while displaying the visual indication of the current level of environmental noise, detecting, via the one or more audio devices, a change in the current level of environmental noise; and in response to detecting the change in the current level of environmental noise, updating display, via the display generation component, of the visual indication of the current level of environmental noise to correspond to the current level of environmental noise.

In some embodiments, a computer system is described. The computer system is configured to communicate with a display generation component and one or more audio devices, comprising: means, during a process to perform a hearing test, for: displaying, via the display generation component, a visual indication of a current level of environmental noise, wherein the visual indication visually changes as the current level of environmental noise changes; while displaying the visual indication of the current level of environmental noise, detecting, via the one or more audio devices, a change in the current level of environmental noise; and in response to detecting the change in the current level of environmental noise, updating display, via the display generation component, of the visual indication of the current level of environmental noise to correspond to the current level of environmental noise.

In some embodiments, a computer program product is described. The computer program product comprises one or more programs configured to be executed by one or more processors of a computer system that is in communication with a display generation component and one or more audio devices, the one or more programs including instructions for: during a process to perform a hearing test: displaying, via the display generation component, a visual indication of a current level of environmental noise, wherein the visual indication visually changes as the current level of environmental noise changes; while displaying the visual indication of the current level of environmental noise, detecting, via the one or more audio devices, a change in the current level of environmental noise; and in response to detecting the change in the current level of environmental noise, updating display, via the display generation component, of the visual indication of the current level of environmental noise to correspond to the current level of environmental noise.

In accordance with some embodiments, a method is described. The method comprises: at a computer system that is in communication with one or more display generation components, one or more input devices, and one or more audio devices: during a process to perform a hearing test: displaying, via the one or more display generation components, a user interface that includes a plurality of discrete volume indicators and a visual indication of a current volume for outputting audio during the hearing test, wherein the plurality of discrete volume indicators include: a volume indicator that corresponds to a minimum volume for outputting audio during the hearing test, a volume indicator that corresponds to a maximum volume for outputting audio during the hearing test, and a volume indicator that corresponds to the current volume for outputting audio during the hearing test; while displaying the user interface that includes the plurality of discrete volume indicators and the visual indication of a current volume for outputting audio during the hearing test, outputting, via a first speaker of the one or more audio devices, a first audio with a first volume; while outputting, via the first speaker of the one or more audio devices, the first audio, detecting, via the one or more input devices, a user input directed to the user interface; and in response to detecting the user input directed to the user interface: changing, based on the user input, a volume of the first audio from the first volume to a second volume that is different from the first volume; and updating, via the one or more display generation components, the user interface to indicate that the current volume for outputting audio during the hearing test is the second volume.

In accordance with some embodiments, a non-transitory computer-readable storage medium is described. The non-transitory computer-readable storage medium stores one or more programs configured to be executed by one or more processors of a computer system that is in communication with one or more display generation components, one or more input devices, and one or more audio devices, the one or more programs including instructions for: during a process to perform a hearing test: displaying, via the one or more display generation components, a user interface that includes a plurality of discrete volume indicators and a visual indication of a current volume for outputting audio during the hearing test, wherein the plurality of discrete volume indicators include: a volume indicator that corresponds to a minimum volume for outputting audio during the hearing test, a volume indicator that corresponds to a maximum volume for outputting audio during the hearing test, and a volume indicator that corresponds to the current volume for outputting audio during the hearing test; while displaying the user interface that includes the plurality of discrete volume indicators and the visual indication of a current volume for outputting audio during the hearing test, outputting, via a first speaker of the one or more audio devices, a first audio with a first volume; and while outputting, via the first speaker of the one or more audio devices, the first audio, detecting, via the one or more input devices, a user input directed to the user interface; and in response to detecting the user input directed to the user interface: changing, based on the user input, a volume of the first audio from the first volume to a second volume that is different from the first volume; and updating, via the one or more display generation components, the user interface to indicate that the current volume for outputting audio during the hearing test is the second volume.

In accordance with some embodiments, a transitory computer-readable storage medium is described. The transitory computer-readable storage medium stores one or more programs configured to be executed by one or more processors of a computer system that is in communication with one or more display generation components, one or more input devices, and one or more audio devices, the one or more programs including instructions for: during a process to perform a hearing test: displaying, via the one or more display generation components, a user interface that includes a plurality of discrete volume indicators and a visual indication of a current volume for outputting audio during the hearing test, wherein the plurality of discrete volume indicators include: a volume indicator that corresponds to a minimum volume for outputting audio during the hearing test, a volume indicator that corresponds to a maximum volume for outputting audio during the hearing test, and a volume indicator that corresponds to the current volume for outputting audio during the hearing test; while displaying the user interface that includes the plurality of discrete volume indicators and the visual indication of a current volume for outputting audio during the hearing test, outputting, via a first speaker of the one or more audio devices, a first audio with a first volume; and while outputting, via the first speaker of the one or more audio devices, the first audio, detecting, via the one or more input devices, a user input directed to the user interface; and in response to detecting the user input directed to the user interface: changing, based on the user input, a volume of the first audio from the first volume to a second volume that is different from the first volume; and updating, via the one or more display generation components, the user interface to indicate that the current volume for outputting audio during the hearing test is the second volume.

In accordance with some embodiments, a computer system is described. The computer system is configured to communicate with one or more display generation components, one or more input devices, and one or more audio devices, and comprises: one or more processors; and memory storing one or more programs configured to be executed by the one or more processors, the one or more programs including instructions for: during a process to perform a hearing test: displaying, via the one or more display generation components, a user interface that includes a plurality of discrete volume indicators and a visual indication of a current volume for outputting audio during the hearing test, wherein the plurality of discrete volume indicators include: a volume indicator that corresponds to a minimum volume for outputting audio during the hearing test, a volume indicator that corresponds to a maximum volume for outputting audio during the hearing test, and a volume indicator that corresponds to the current volume for outputting audio during the hearing test; while displaying the user interface that includes the plurality of discrete volume indicators and the visual indication of a current volume for outputting audio during the hearing test, outputting, via a first speaker of the one or more audio devices, a first audio with a first volume; and while outputting, via the first speaker of the one or more audio devices, the first audio, detecting, via the one or more input devices, a user input directed to the user interface; and in response to detecting the user input directed to the user interface: changing, based on the user input, a volume of the first audio from the first volume to a second volume that is different from the first volume; and updating, via the one or more display generation components, the user interface to indicate that the current volume for outputting audio during the hearing test is the second volume.

In accordance with some embodiments, a computer system is described. The computer system is configured to communicate with one or more display generation components, one or more input devices, and one or more audio devices, and comprises: during a process to perform a hearing test: means for displaying, via the one or more display generation components, a user interface that includes a plurality of discrete volume indicators and a visual indication of a current volume for outputting audio during the hearing test, wherein the plurality of discrete volume indicators include: a volume indicator that corresponds to a minimum volume for outputting audio during the hearing test, a volume indicator that corresponds to a maximum volume for outputting audio during the hearing test, and a volume indicator that corresponds to the current volume for outputting audio during the hearing test; means, while displaying the user interface that includes the plurality of discrete volume indicators and the visual indication of a current volume for outputting audio during the hearing test, for outputting, via a first speaker of the one or more audio devices, a first audio with a first volume; and means, while outputting, via the first speaker of the one or more audio devices, the first audio, for detecting, via the one or more input devices, a user input directed to the user interface; and means, responsive to detecting the user input directed to the user interface, for: changing, based on the user input, a volume of the first audio from the first volume to a second volume that is different from the first volume; and updating, via the one or more display generation components, the user interface to indicate that the current volume for outputting audio during the hearing test is the second volume.

In accordance with some embodiments, a computer program product is described. The computer program product comprises one or more programs configured to be executed by one or more processors of a computer system that is in communication with one or more display generation components, one or more input devices, and one or more audio devices, the one or more programs including instructions for: during a process to perform a hearing test: displaying, via the one or more display generation components, a user interface that includes a plurality of discrete volume indicators and a visual indication of a current volume for outputting audio during the hearing test, wherein the plurality of discrete volume indicators include: a volume indicator that corresponds to a minimum volume for outputting audio during the hearing test, a volume indicator that corresponds to a maximum volume for outputting audio during the hearing test, and a volume indicator that corresponds to the current volume for outputting audio during the hearing test; while displaying the user interface that includes the plurality of discrete volume indicators and the visual indication of a current volume for outputting audio during the hearing test, outputting, via a first speaker of the one or more audio devices, a first audio with a first volume; and while outputting, via the first speaker of the one or more audio devices, the first audio, detecting, via the one or more input devices, a user input directed to the user interface; and in response to detecting the user input directed to the user interface: changing, based on the user input, a volume of the first audio from the first volume to a second volume that is different from the first volume; and updating, via the one or more display generation components, the user interface to indicate that the current volume for outputting audio during the hearing test is the second volume.

Executable instructions for performing these functions are, optionally, included in a non-transitory computer-readable storage medium or other computer program product configured for execution by one or more processors. Executable instructions for performing these functions are, optionally, included in a transitory computer-readable storage medium or other computer program product configured for execution by one or more processors.

Thus, devices are provided with faster, more efficient methods and interfaces for managing hearing health, thereby increasing the effectiveness, efficiency, and user satisfaction with such devices. Such methods and interfaces may complement or replace other methods for managing hearing health.

### DESCRIPTION OF THE FIGURES

For a better understanding of the various described embodiments, reference should be made to the Description of Embodiments below, in conjunction with the following drawings in which like reference numerals refer to corresponding parts throughout the figures.

FIG. 1A is a block diagram illustrating a portable multifunction device with a touch-sensitive display in accordance with some embodiments.

FIG. 1B is a block diagram illustrating exemplary components for event handling in accordance with some embodiments.

FIG. 2 illustrates a portable multifunction device having a touch screen in accordance with some embodiments.

FIG. 3A is a block diagram of an exemplary multifunction device with a display and a touch-sensitive surface in accordance with some embodiments.

FIGS. 3B-3G illustrate the use of Application Programming Interfaces (APIs) to perform operations.

FIG. 4A illustrates an exemplary user interface for a menu of applications on a portable multifunction device in accordance with some embodiments.

FIG. 4B illustrates an exemplary user interface for a multifunction device with a touch-sensitive surface that is separate from the display in accordance with some embodiments.

FIG. 5A illustrates a personal electronic device in accordance with some embodiments.

FIG. 5B is a block diagram illustrating a personal electronic device in accordance with some embodiments.

FIGS. 6A-6BH illustrates exemplary devices and user interfaces for managing hearing health, in accordance with some embodiments.

FIG. 7 is a flow diagram illustrating methods of prompting a user to initiate a hearing test, in accordance with some embodiments.

FIG. 8 is a flow diagram illustrating methods of modifying audio characteristics of different types of audio, in accordance with some embodiments.

FIGS. 9A-9D illustrate example user interfaces for displaying a visual indication of a current level of environmental noise, in accordance with some embodiments.

FIG. 10 is a flow diagram illustrating methods of displaying a visual indication of a current level of environmental noise, in accordance with some embodiments.

FIGS. 11A-11N illustrate example user interfaces for performing hearing tests, in accordance with some embodiments.

FIGS. 12A-12B are a flow diagram illustrating methods of performing hearing tests, in accordance with some embodiments.

### DESCRIPTION OF EMBODIMENTS

The following description sets forth exemplary methods, parameters, and the like. It should be recognized, however, that such description is not intended as a limitation on the scope of the present disclosure but is instead provided as a description of exemplary embodiments.

There is a need for electronic devices that provide efficient methods and interfaces for managing hearing health. For example, offering a hearing test to a user during a process to pair headphones with a computer system enables the computer system to quickly and efficiently initiate a hearing test that uses the headphones. For another example, the computer system using user requests to adjusts different types of audio (e.g., environment audio, phone call audio, and/or application audio) enables the computer system to provide audio that is better suited to the user. Such techniques can reduce the cognitive burden on a user who want to manage their hearing health, thereby enhancing productivity. Further, such techniques can reduce processor and battery power otherwise wasted on redundant user inputs.

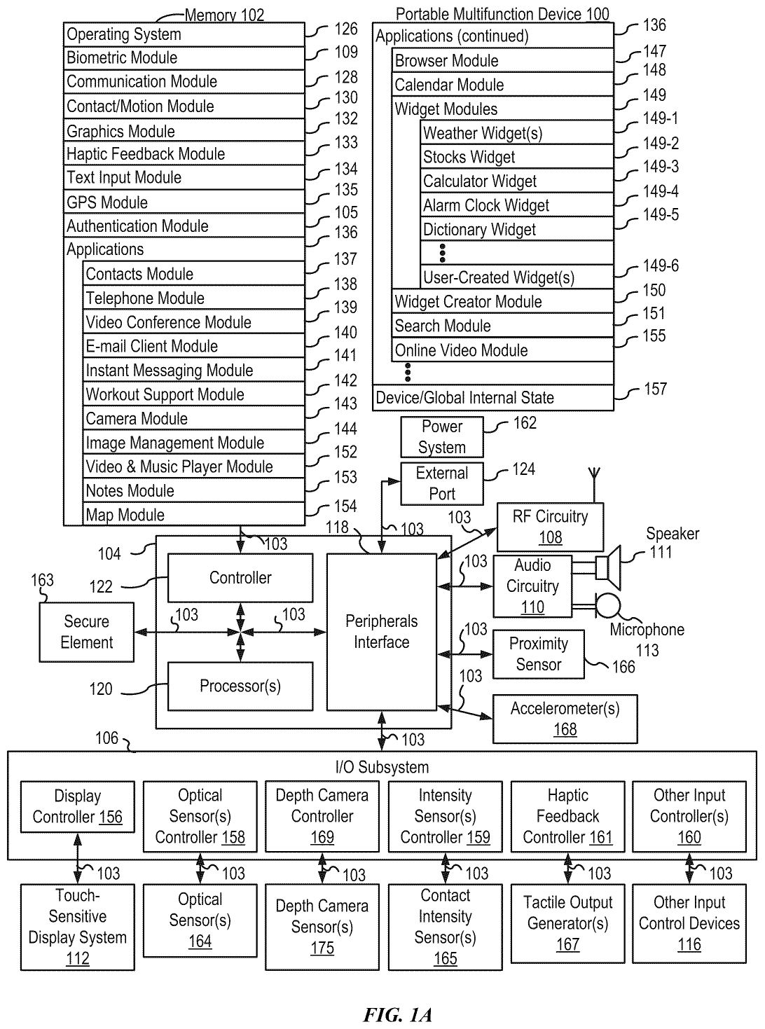

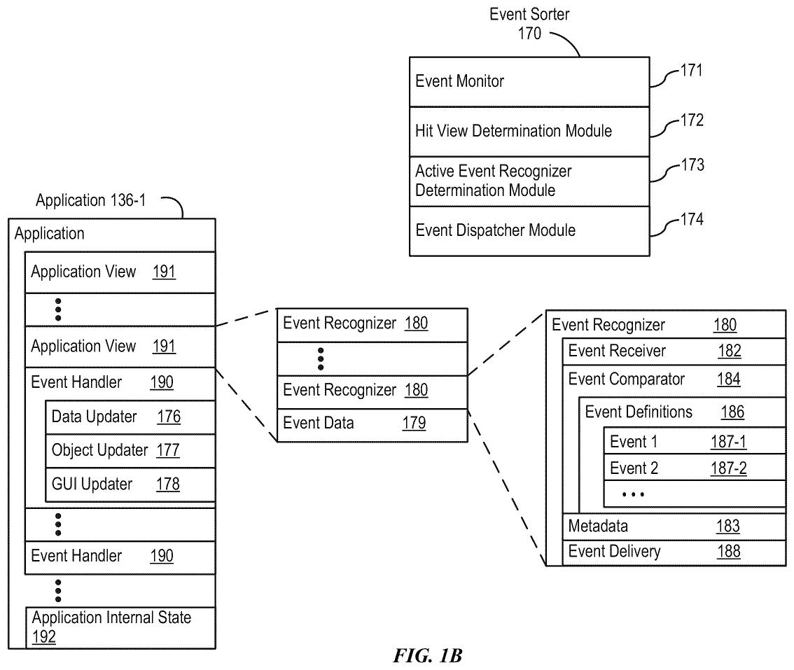

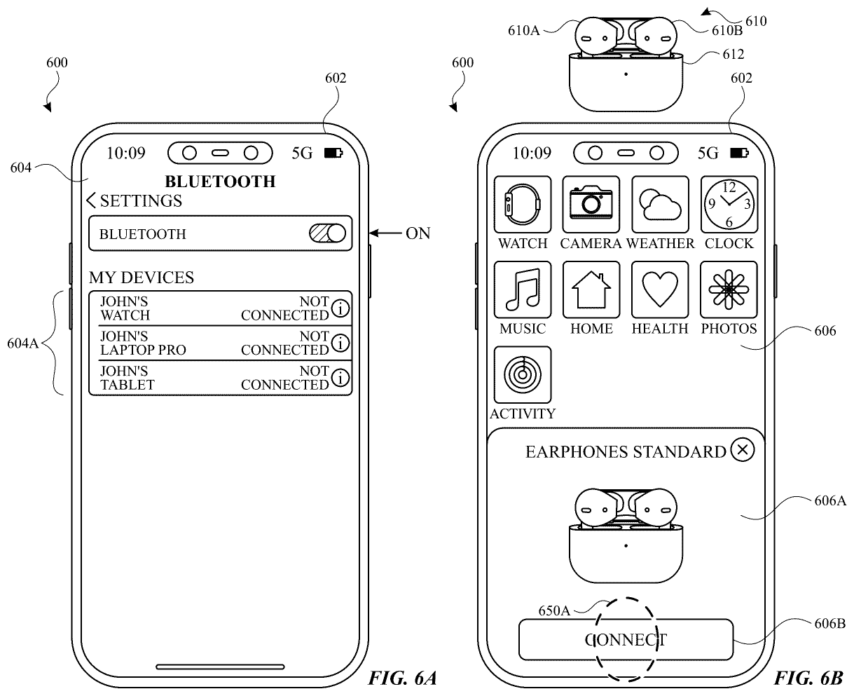

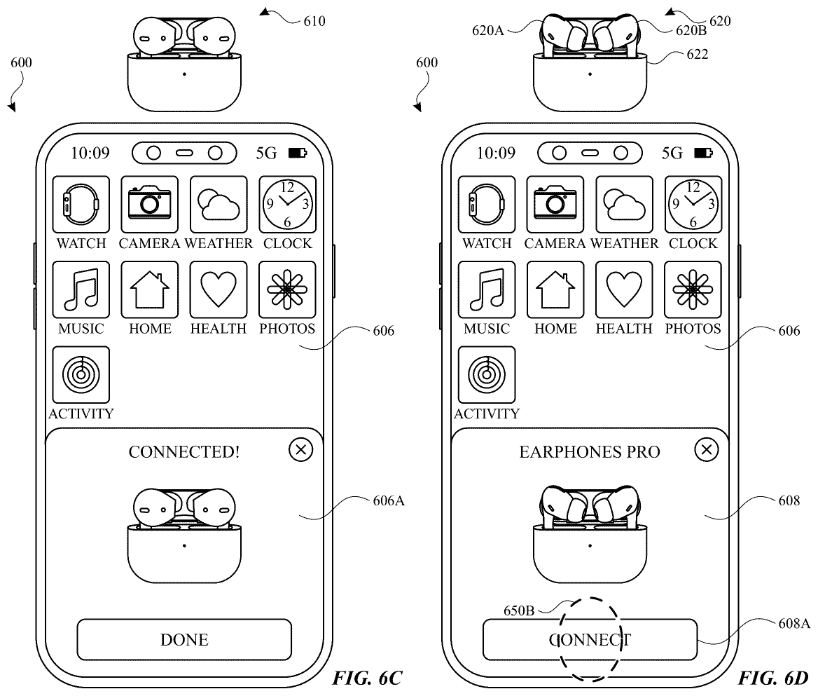

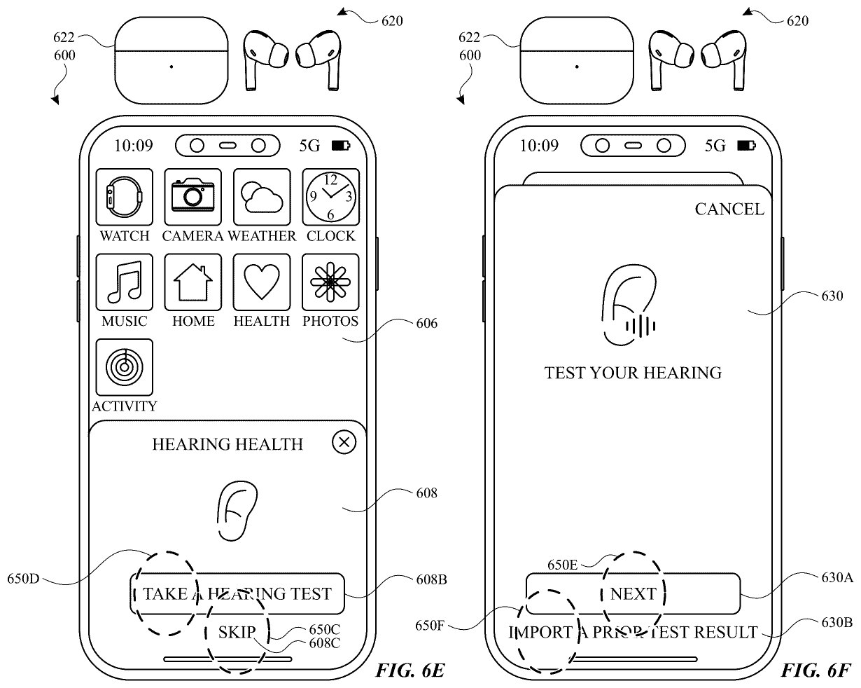

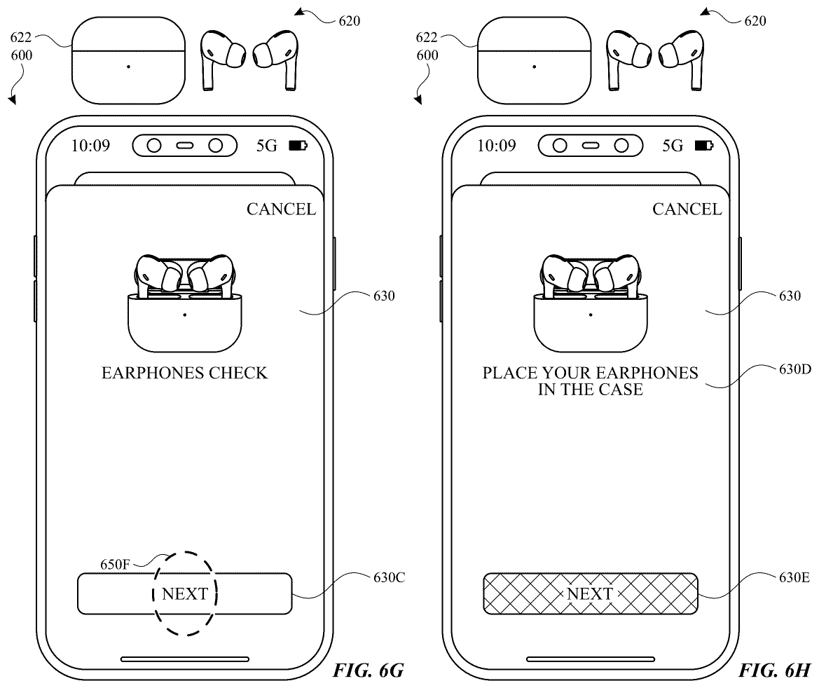

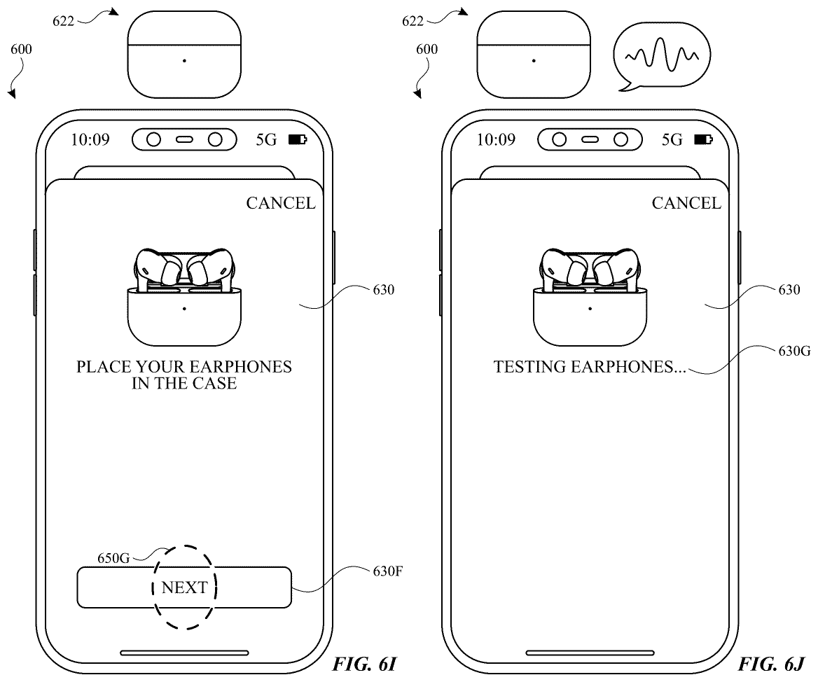

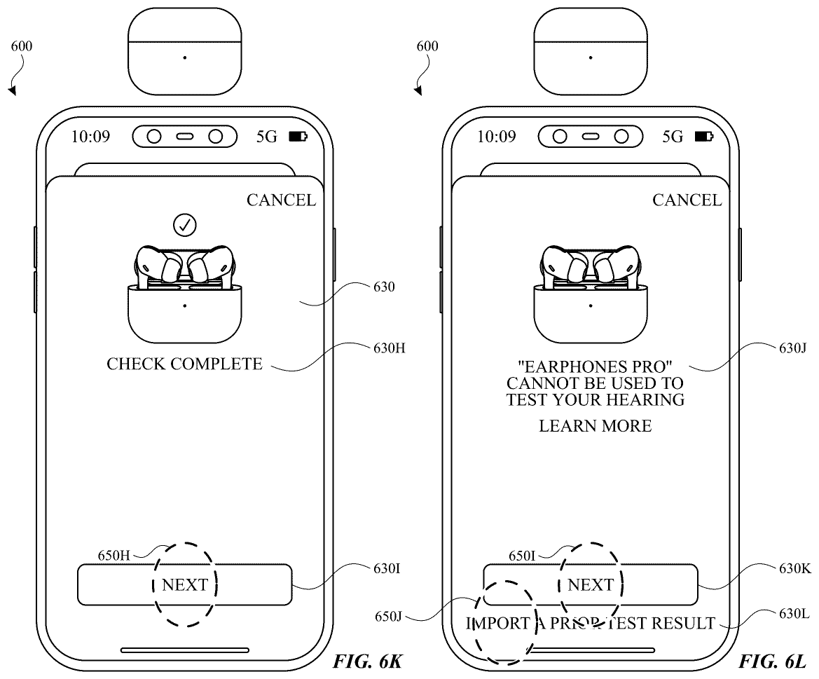

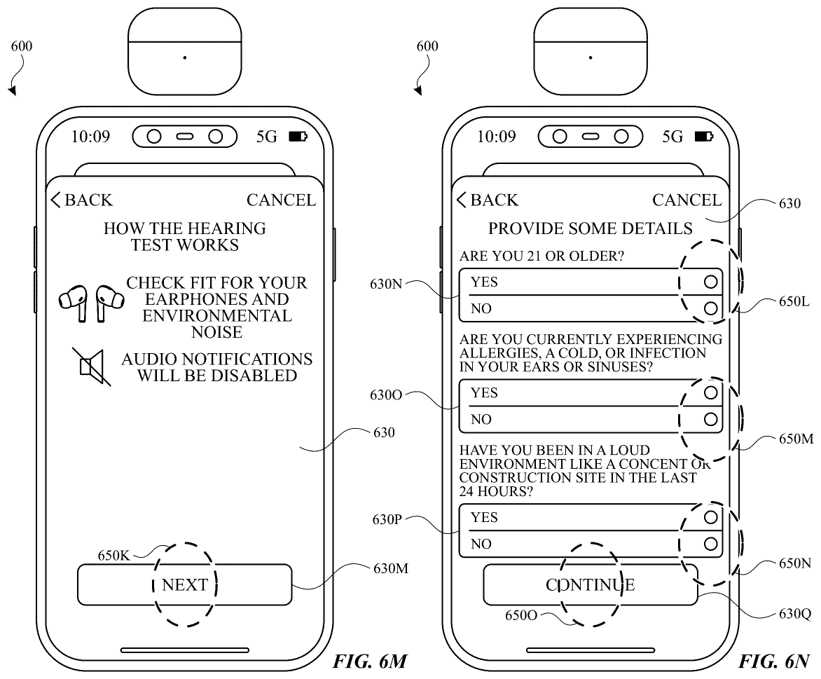

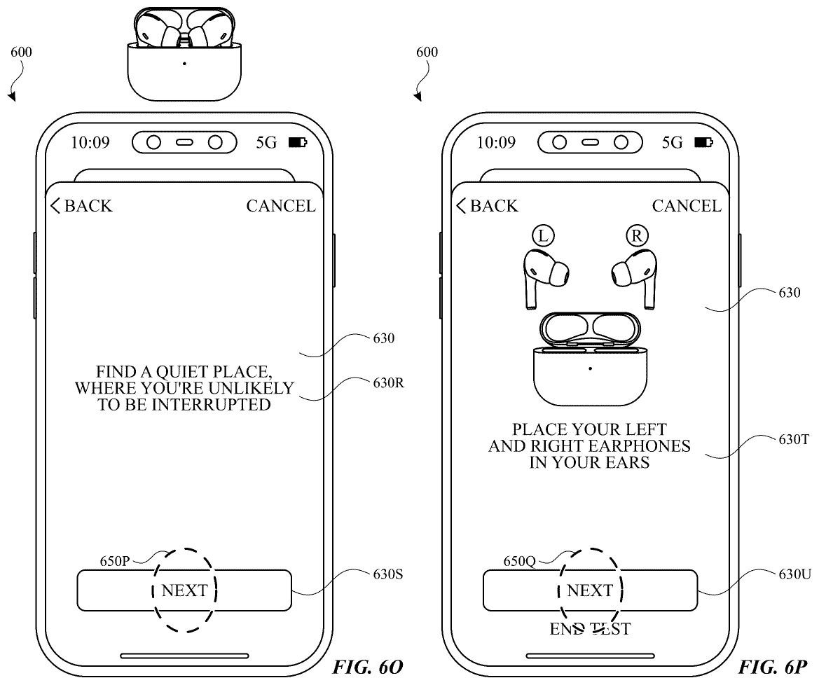

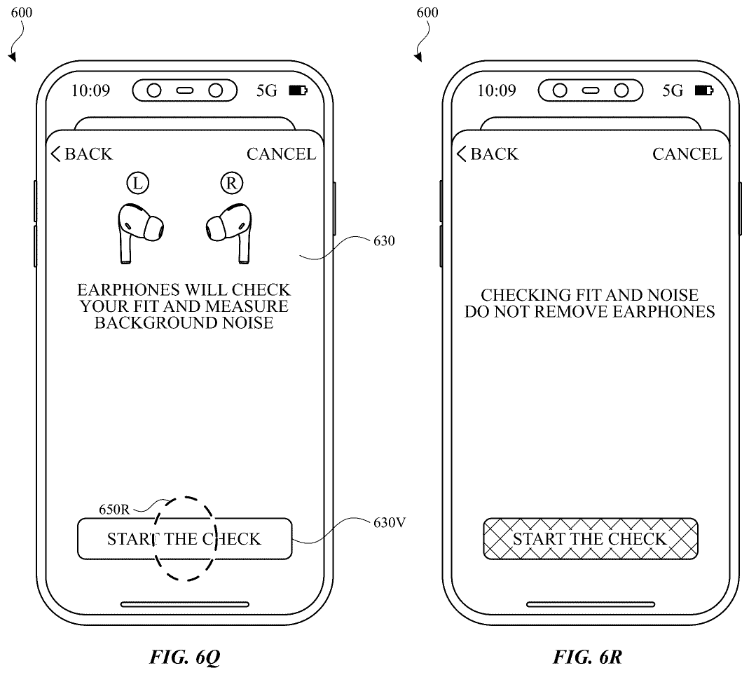

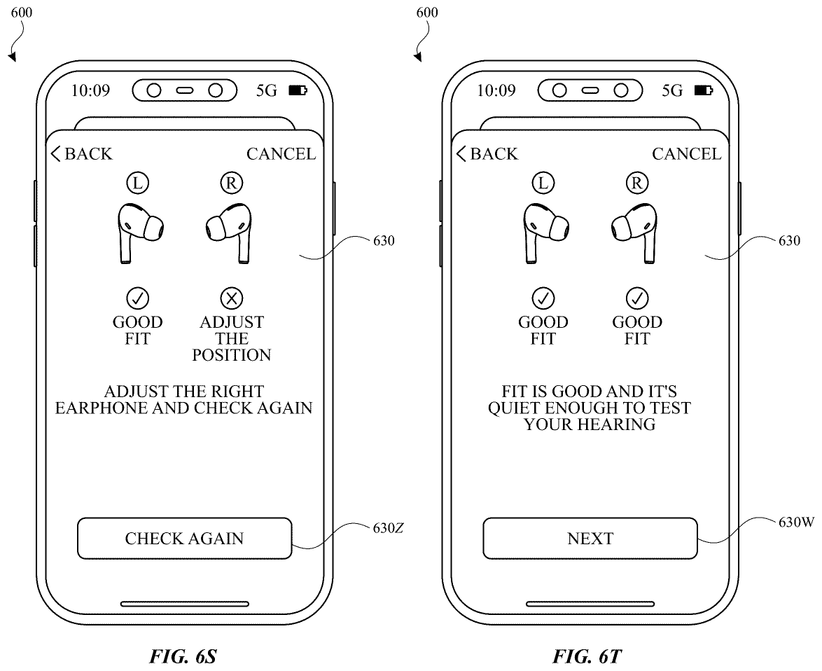

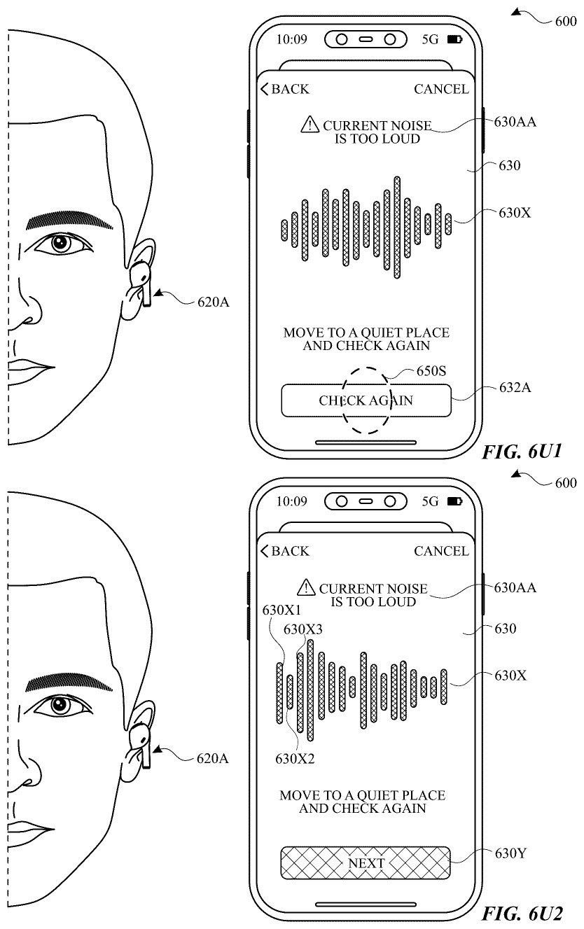

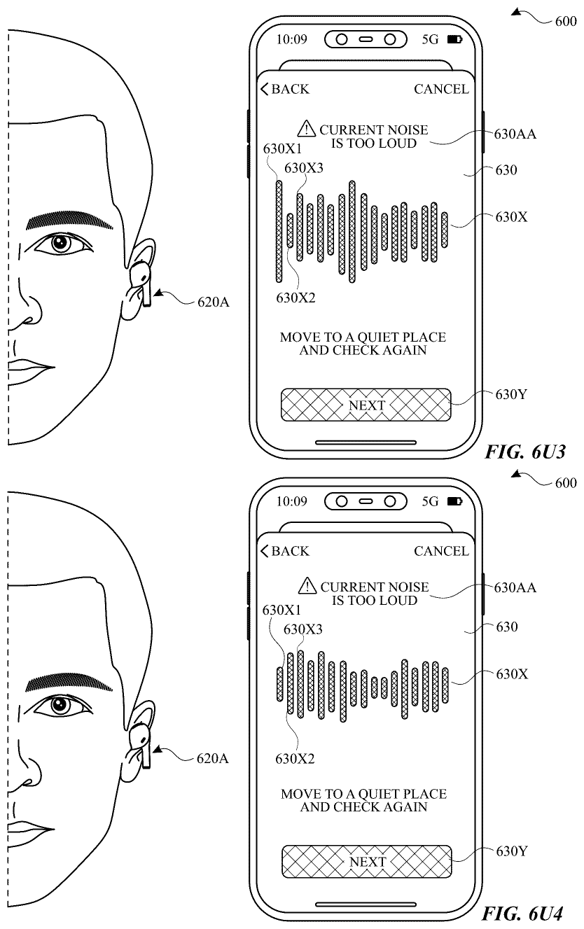

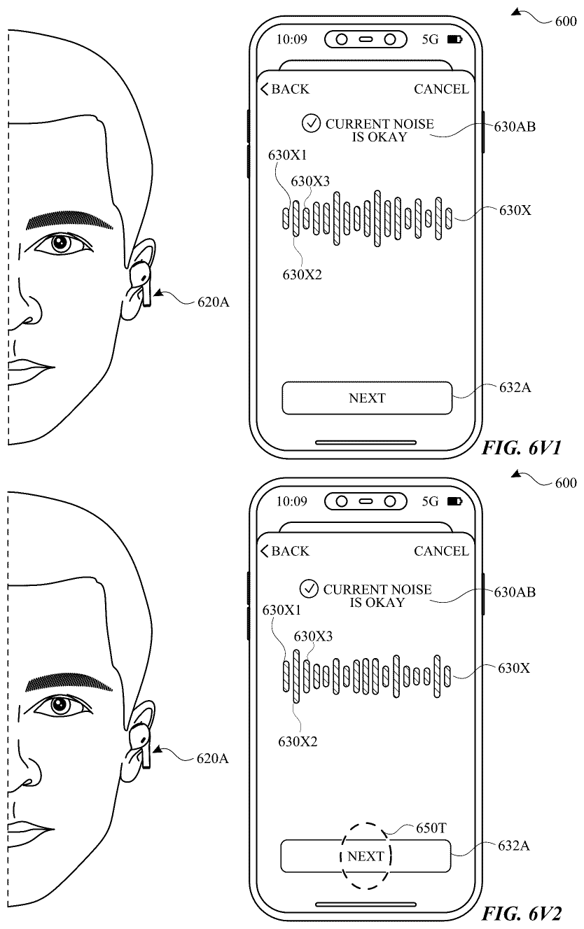

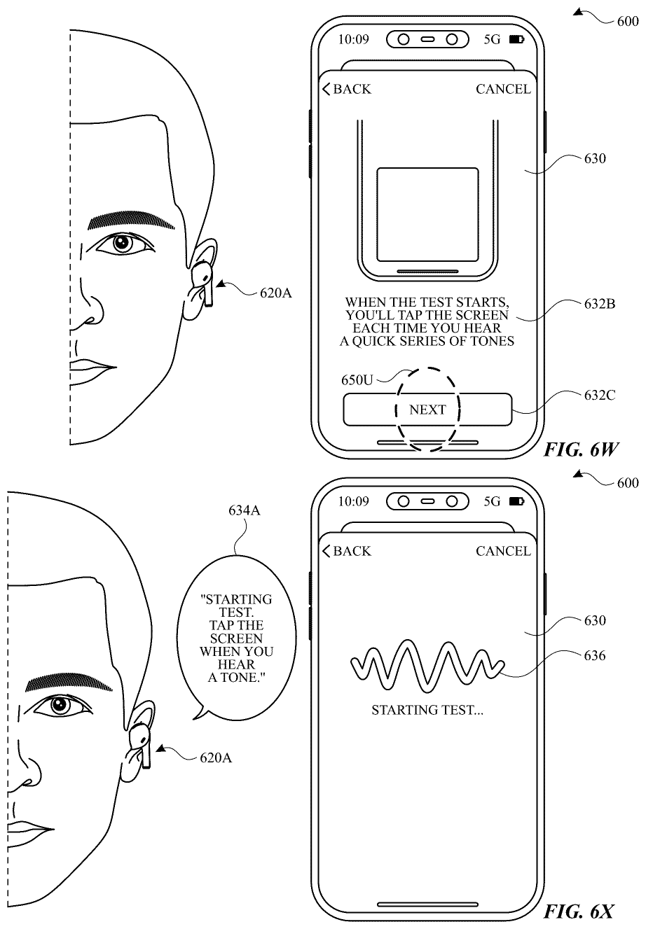

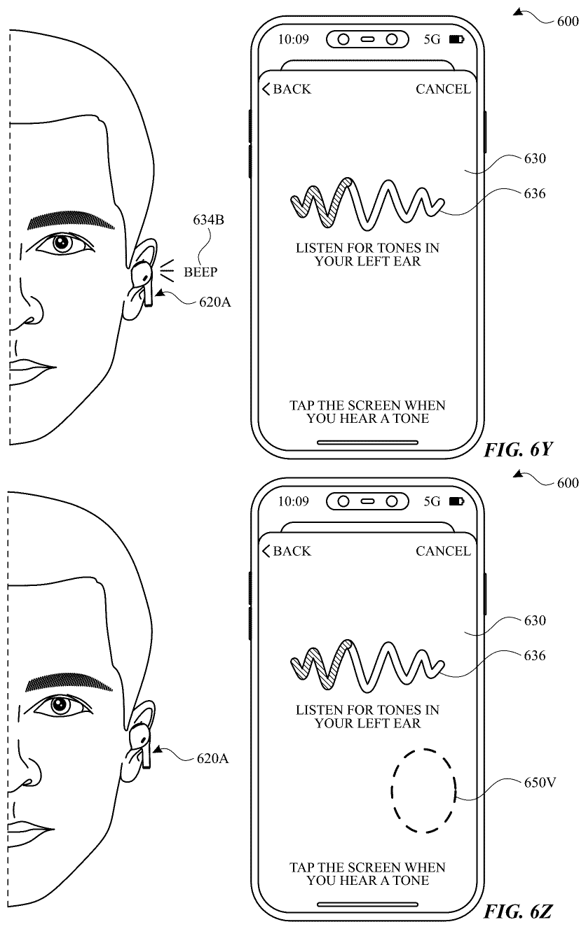

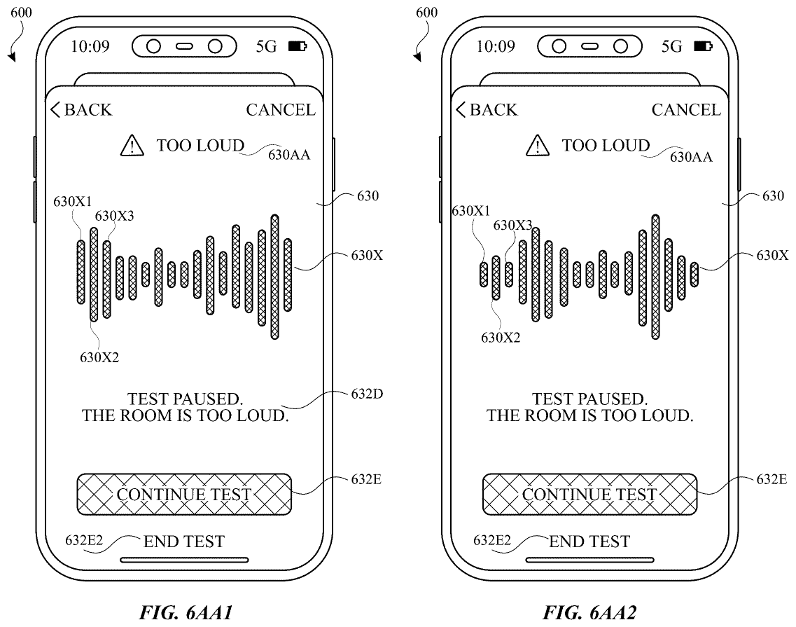

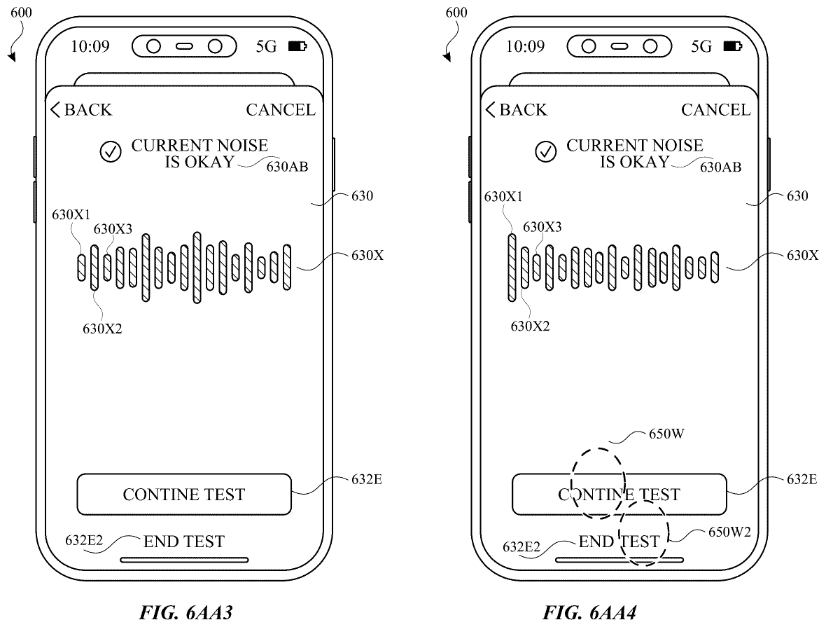

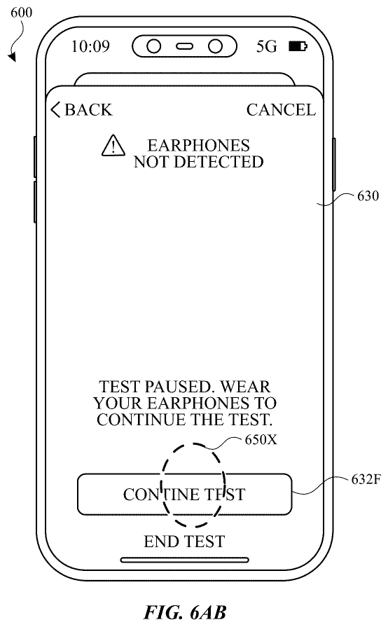

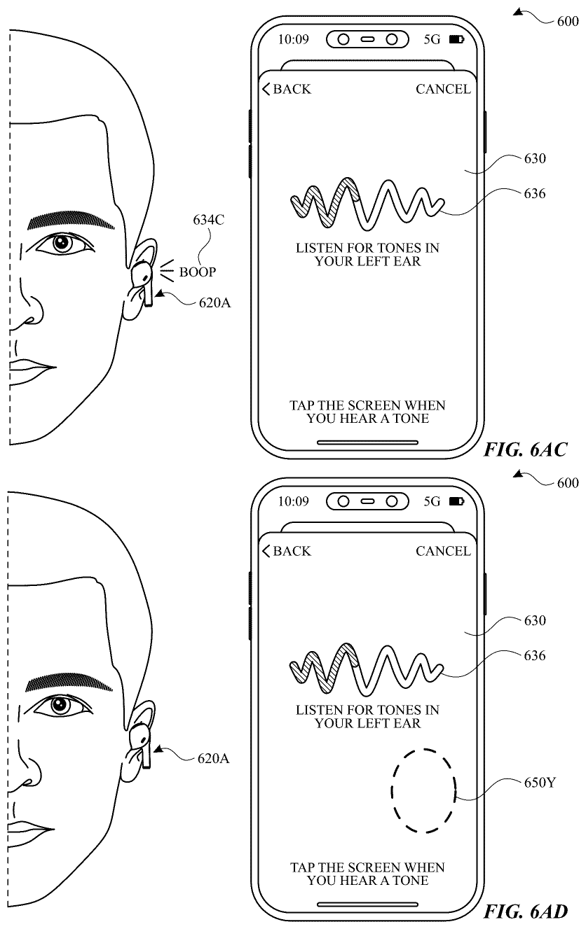

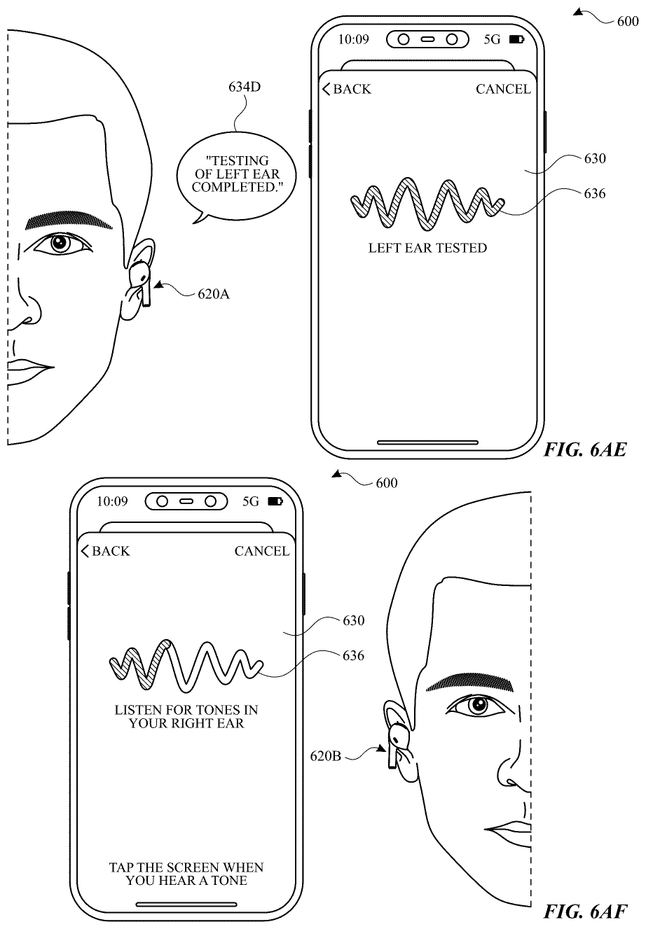

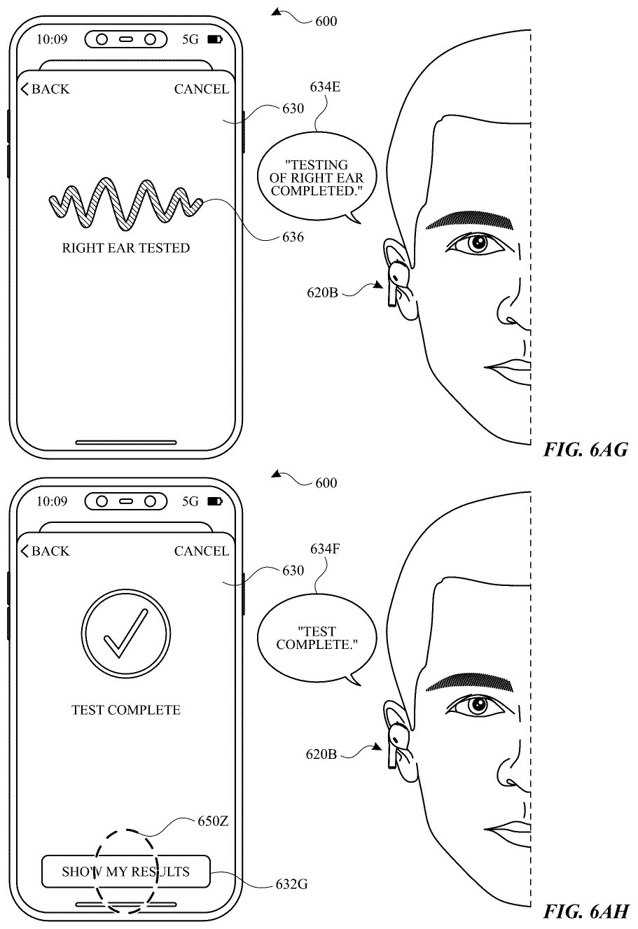

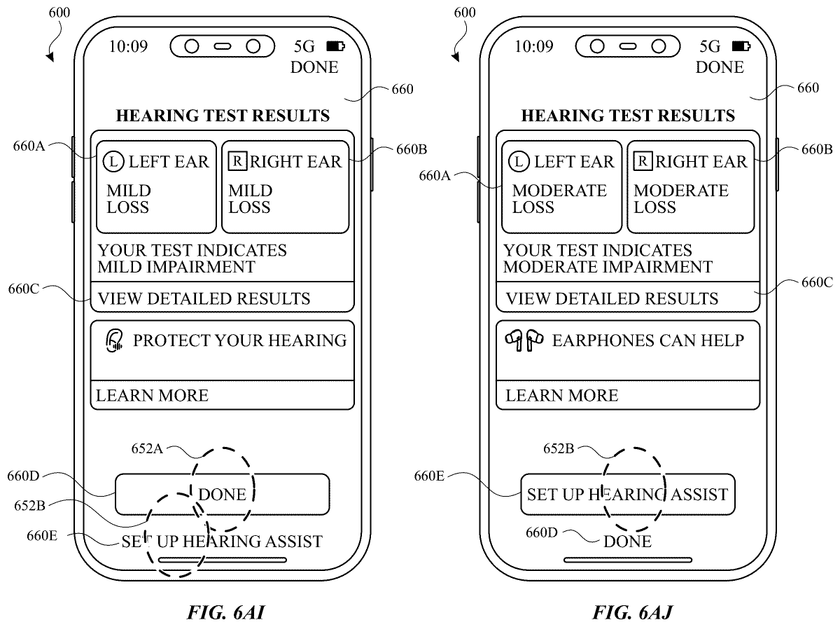

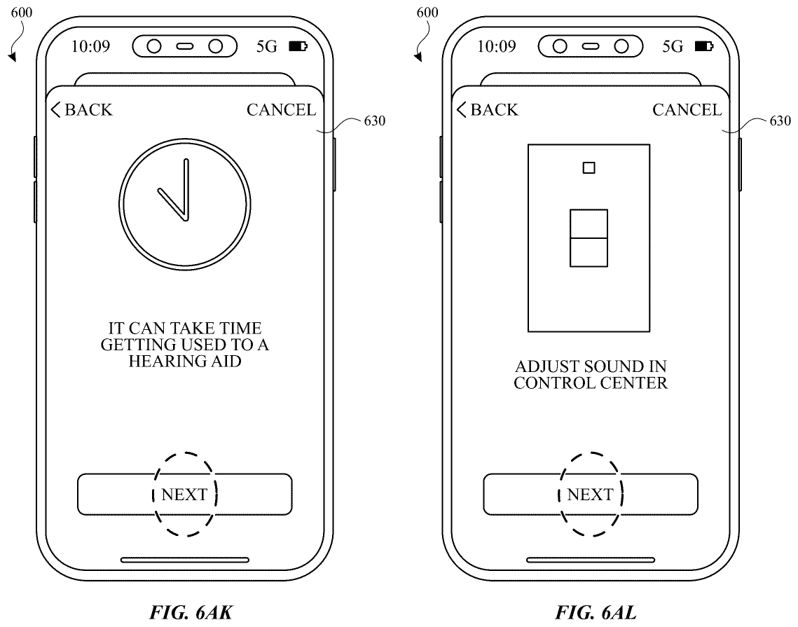

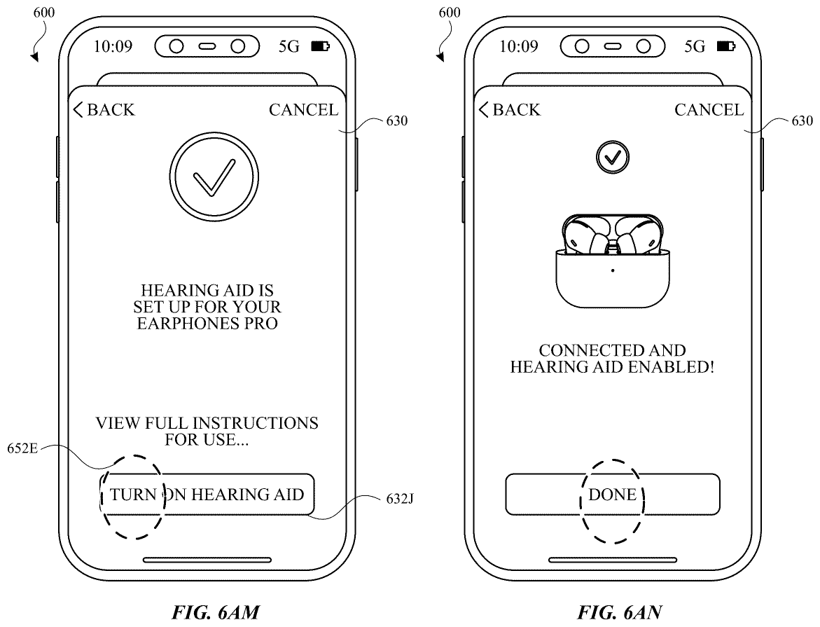

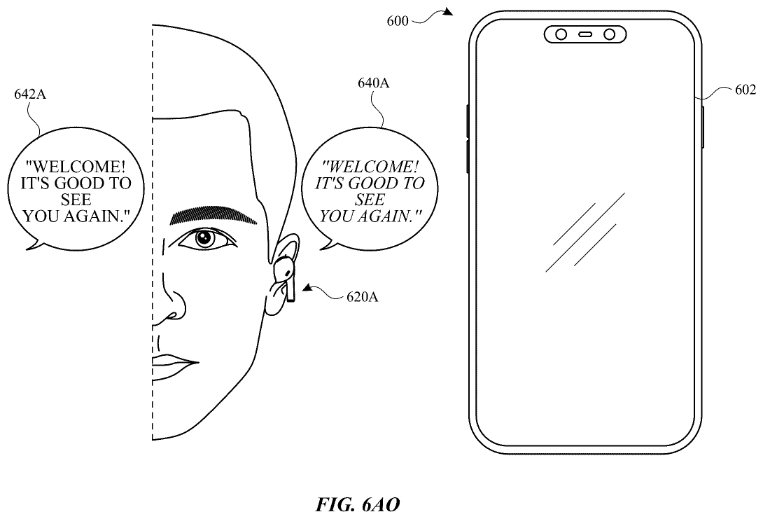

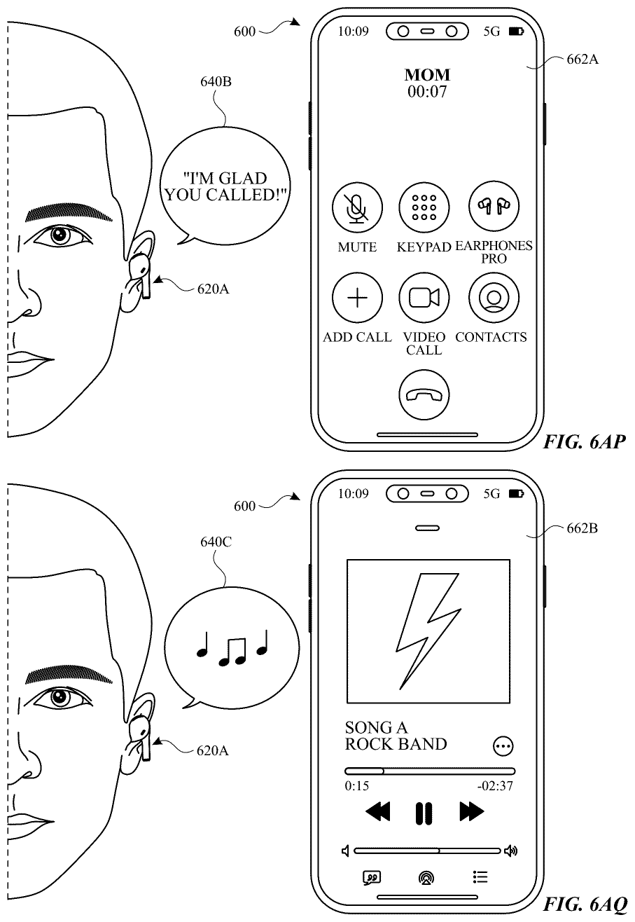

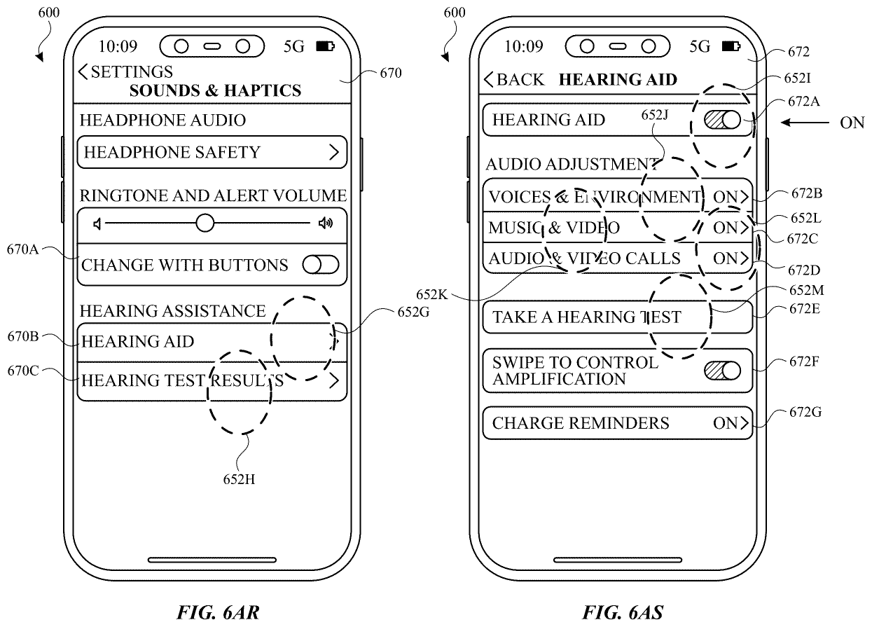

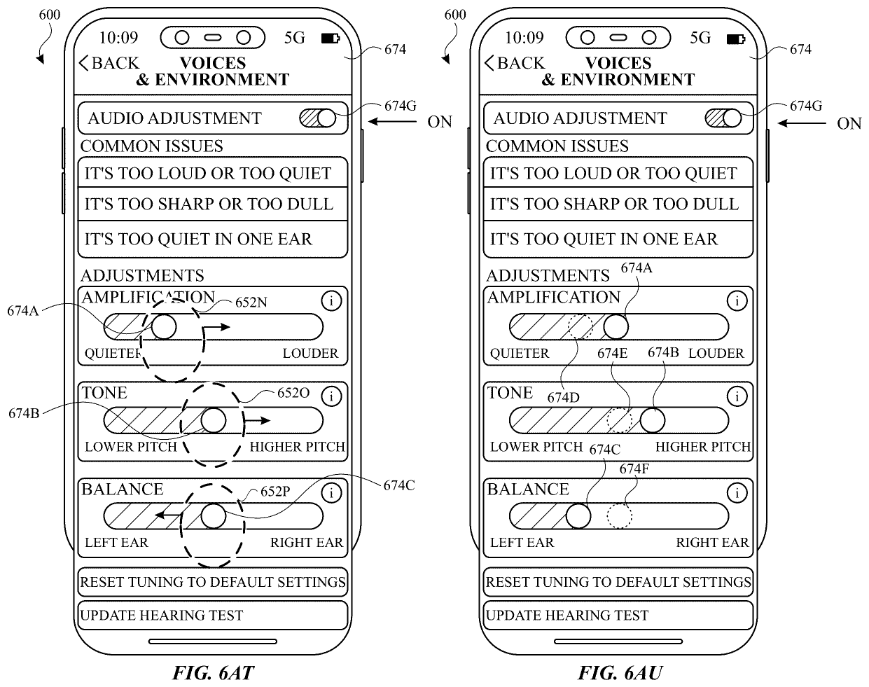

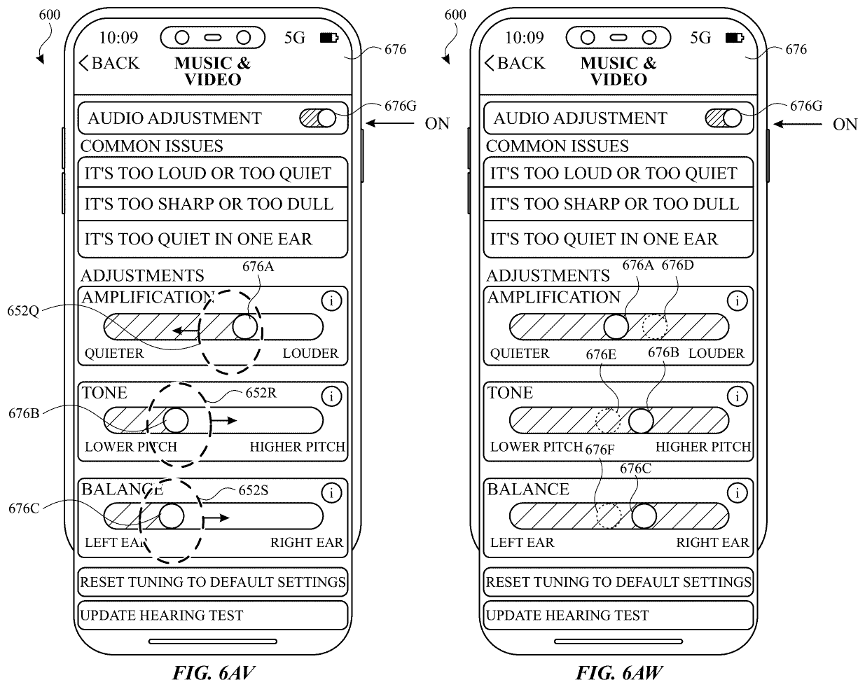

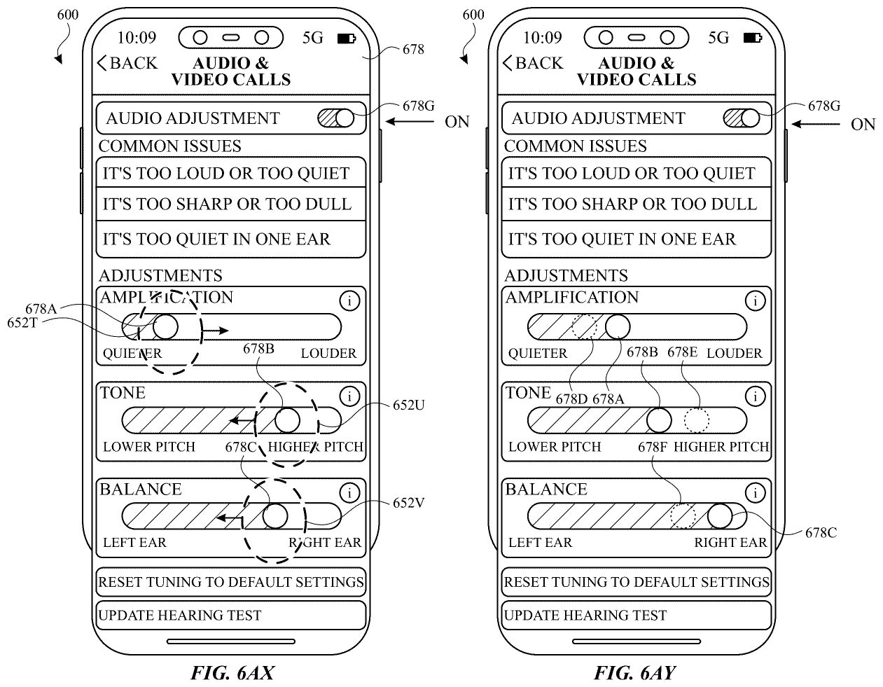

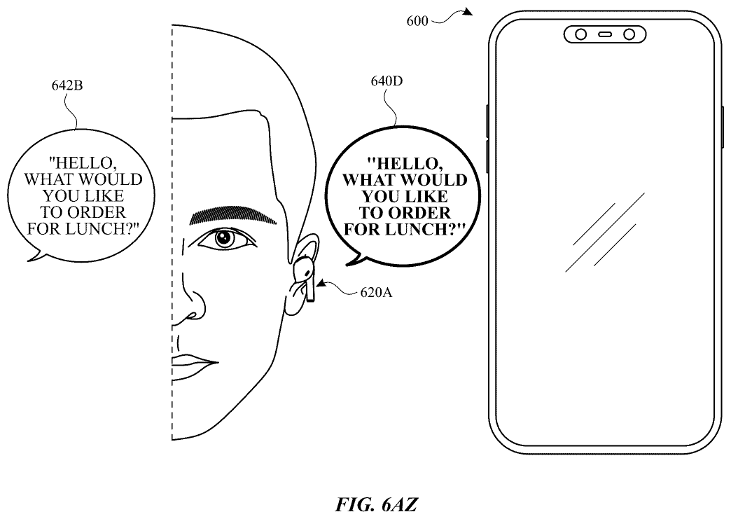

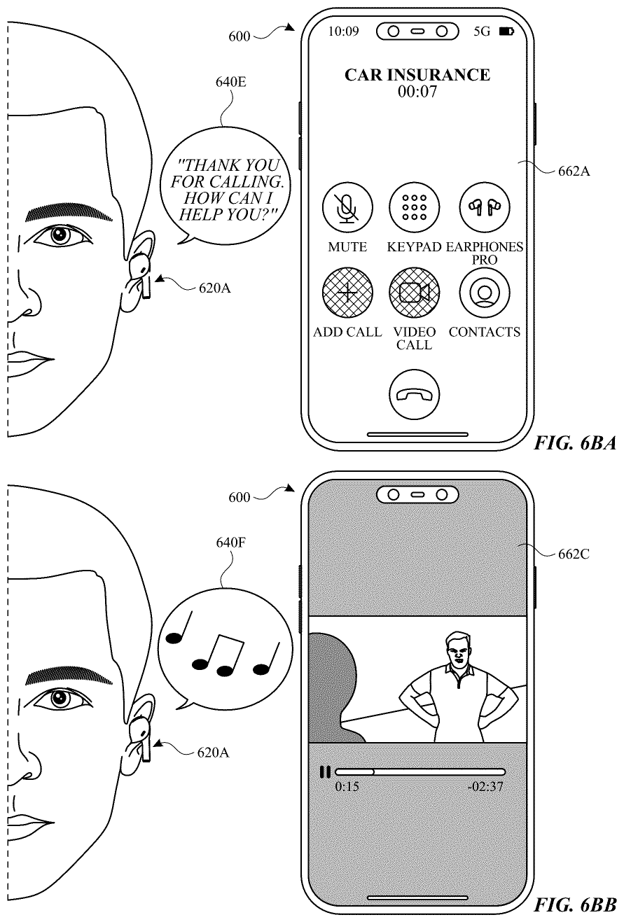

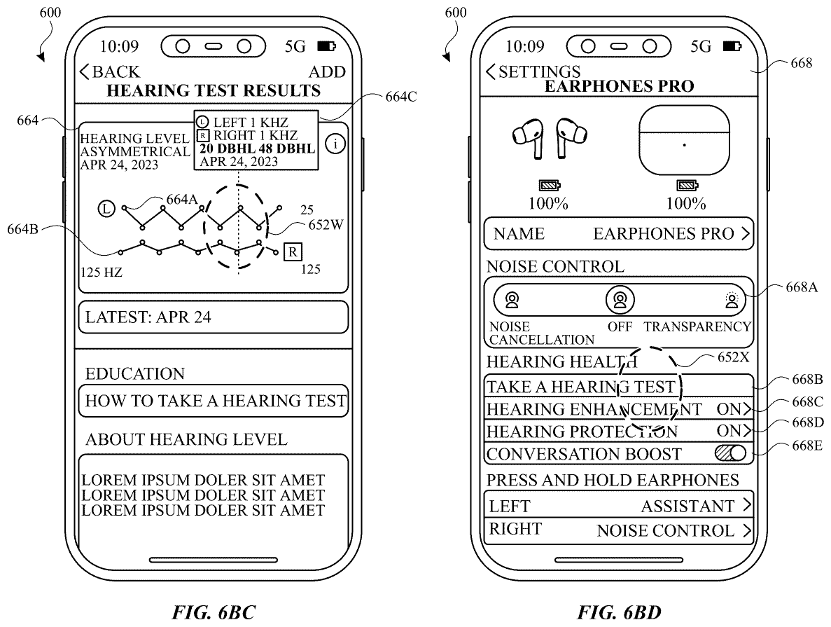

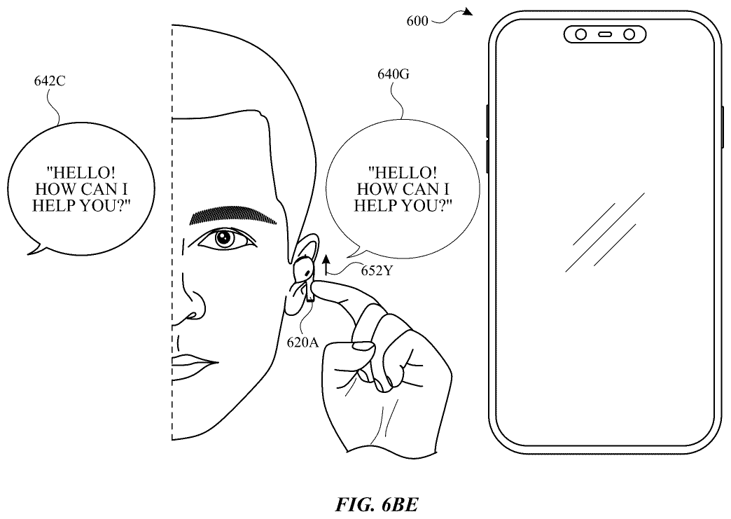

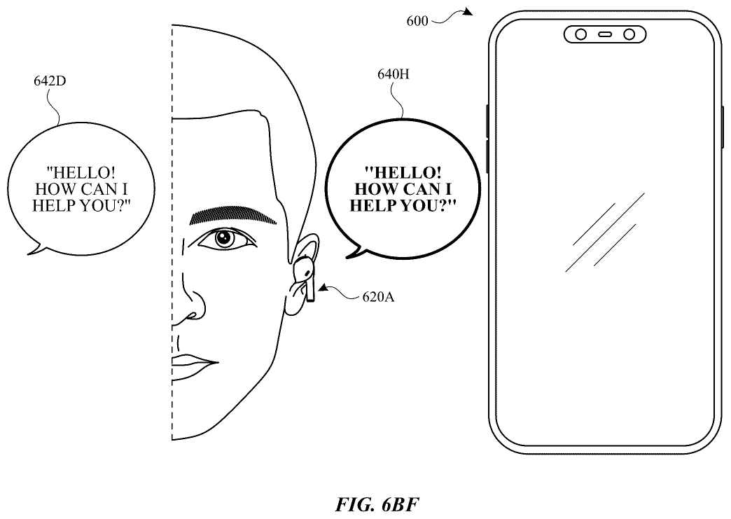

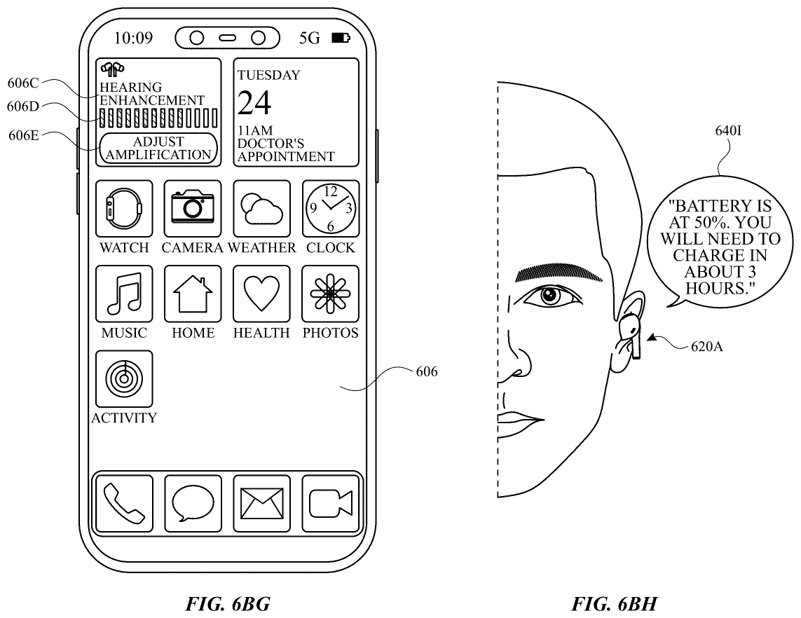

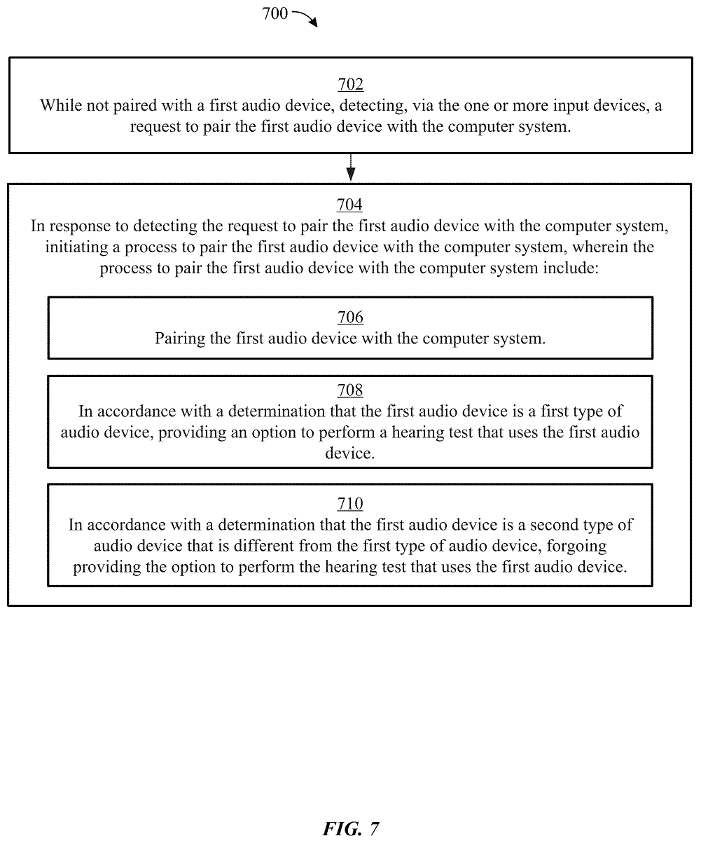

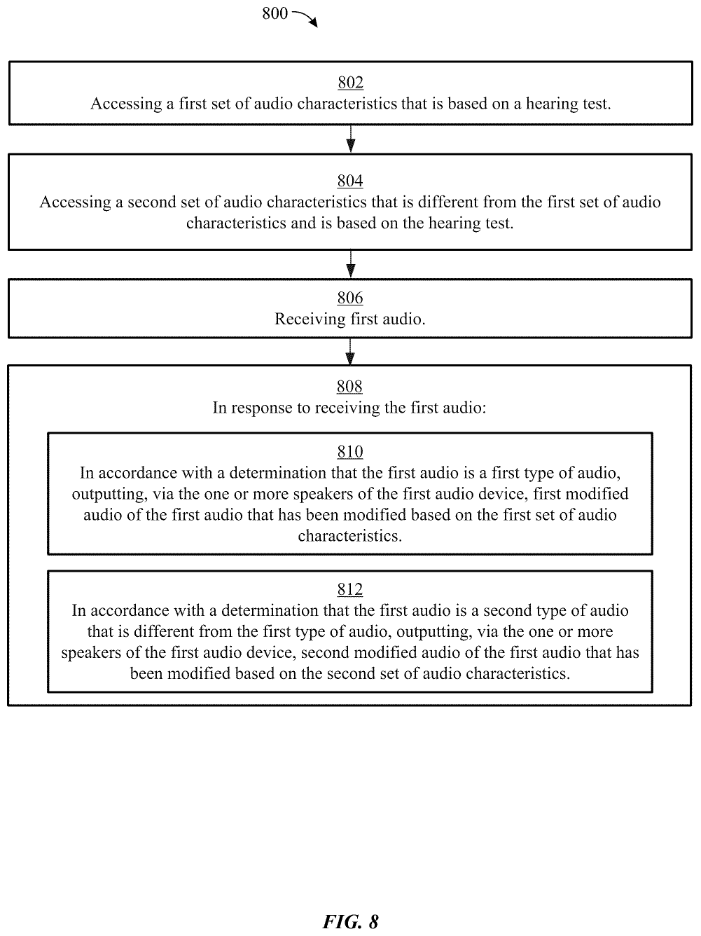

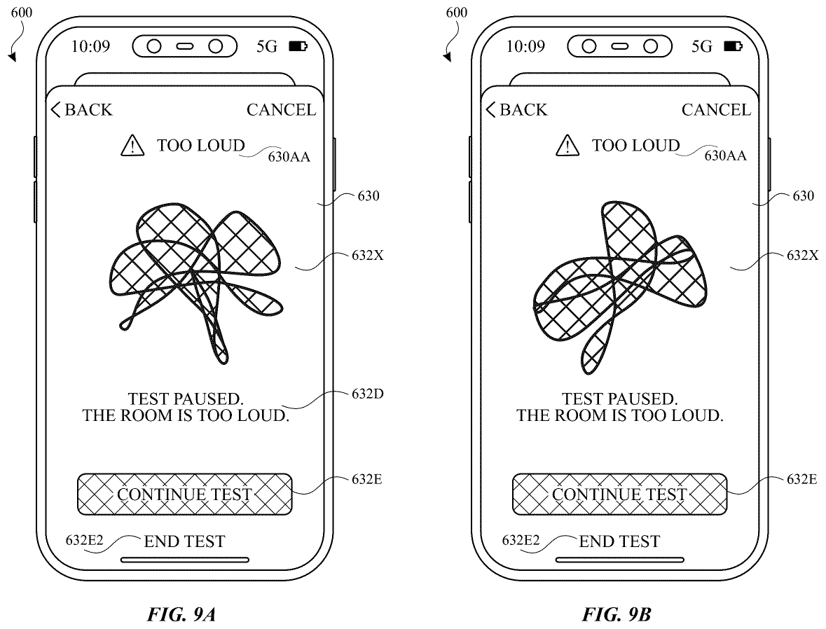

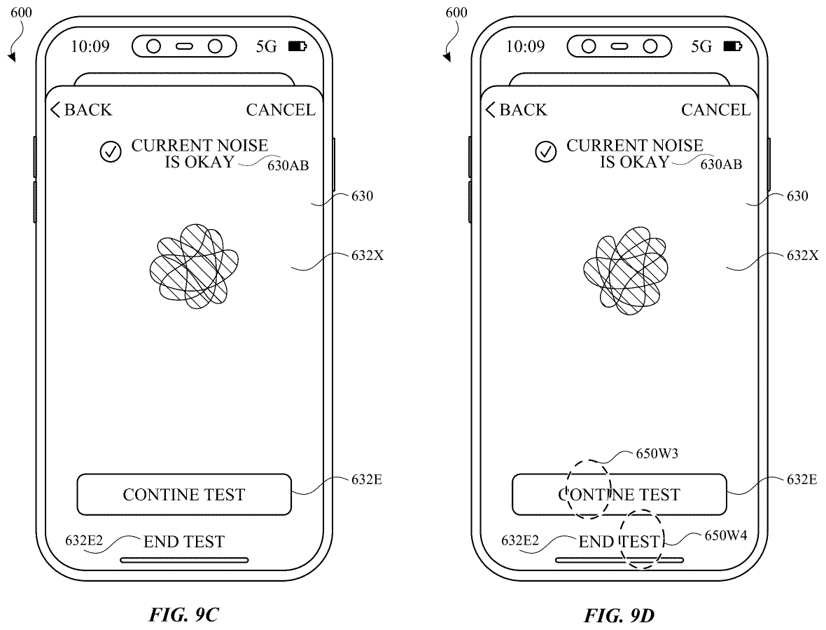

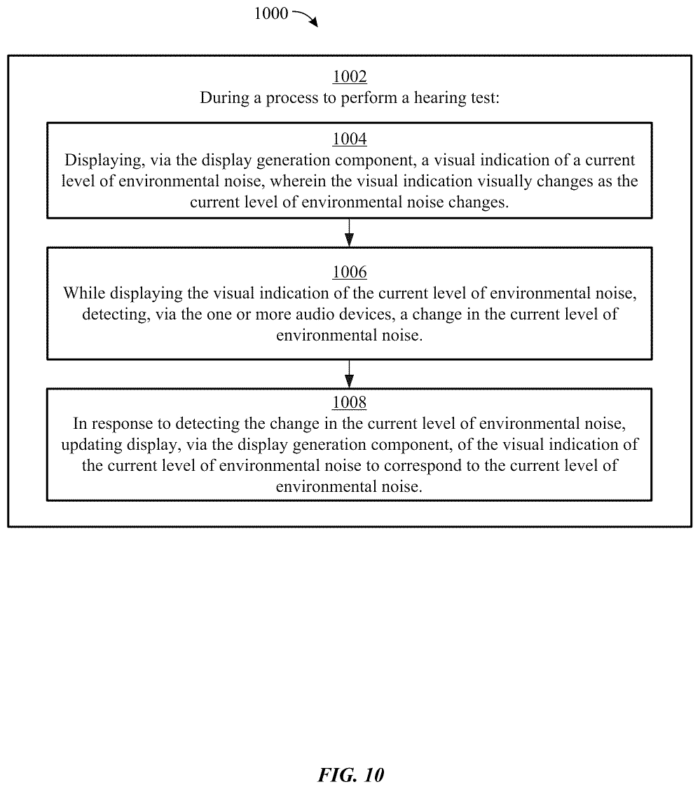

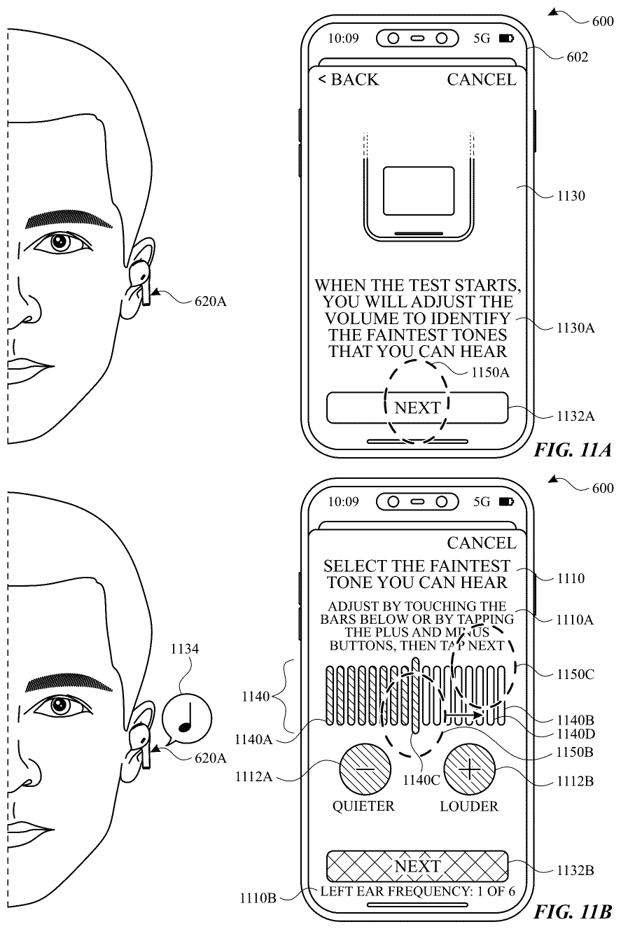

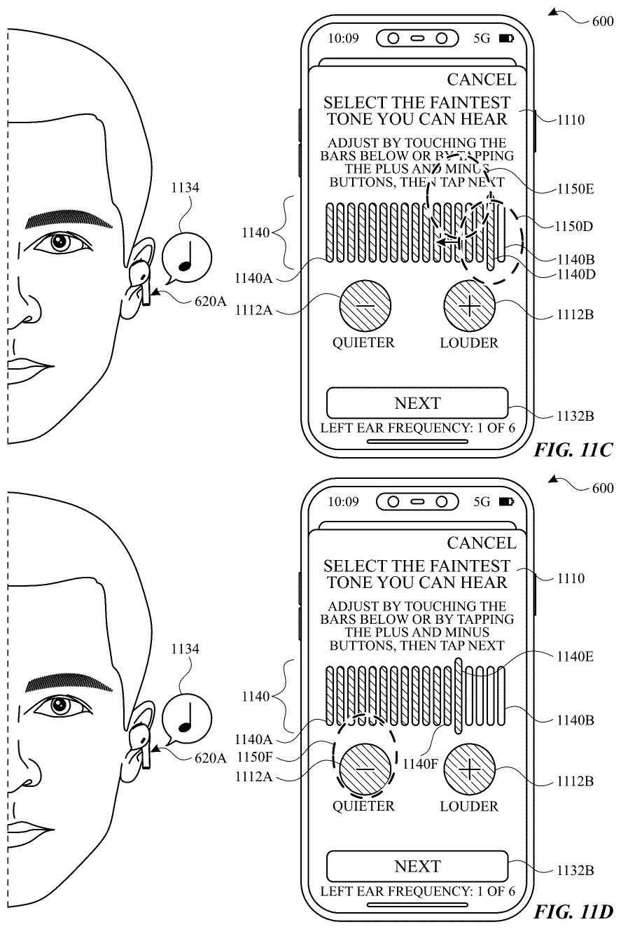

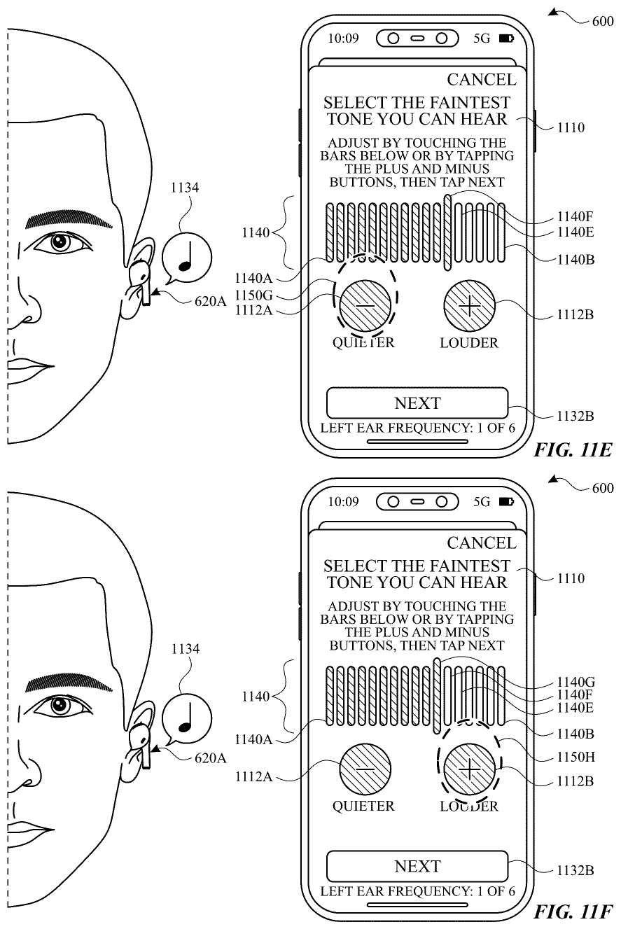

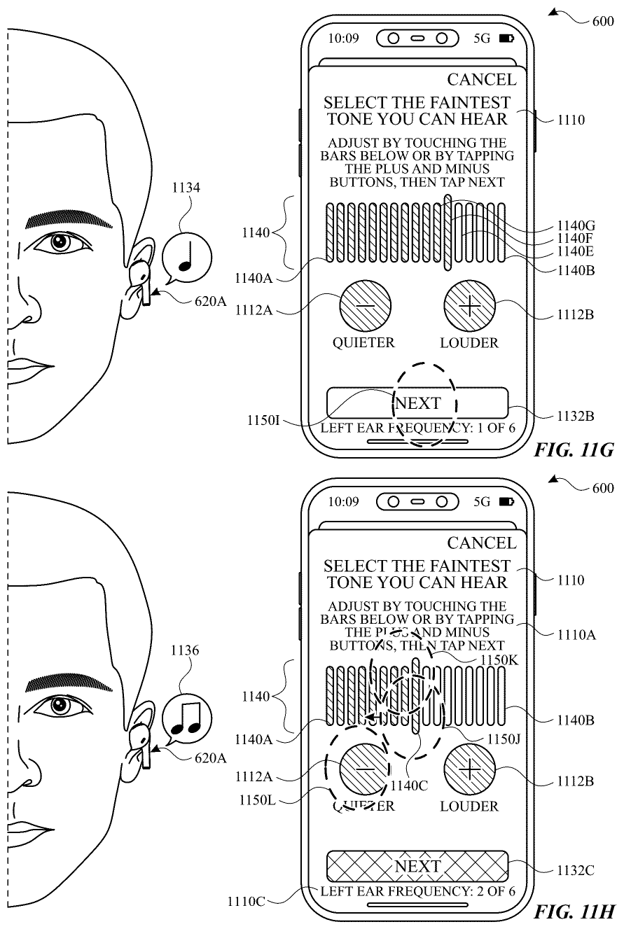

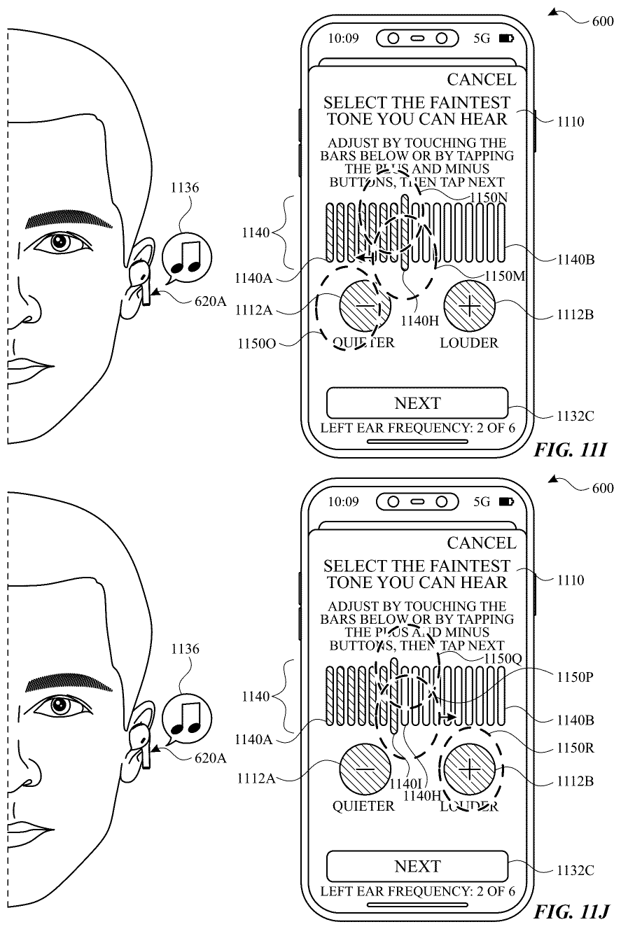

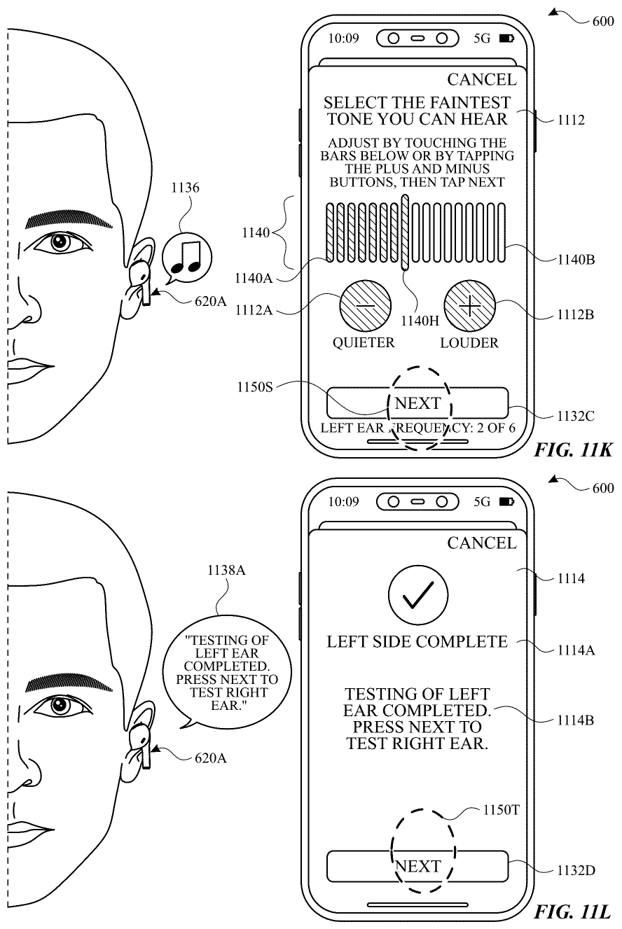

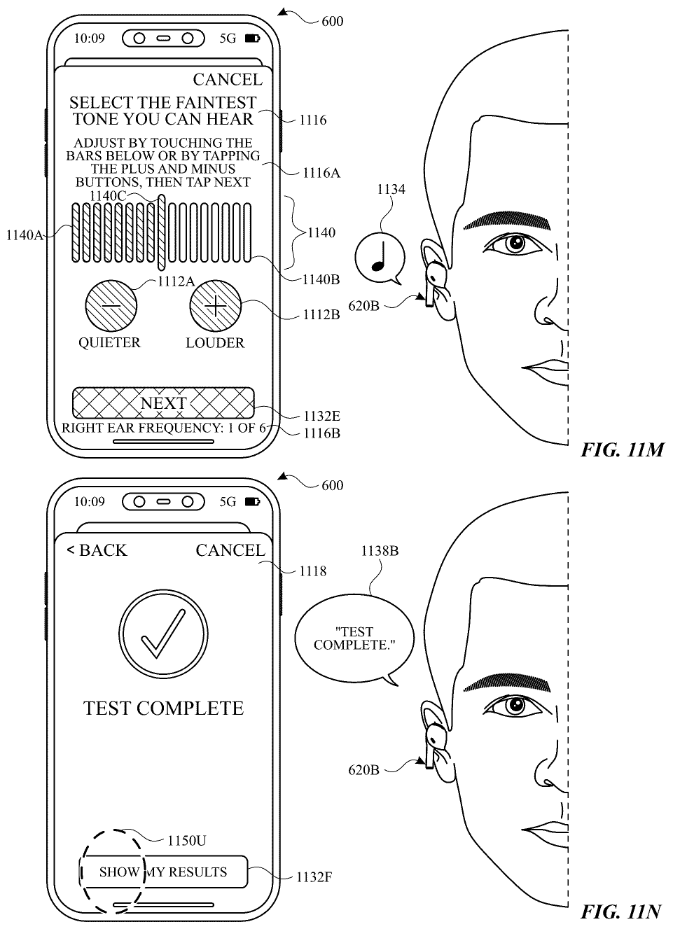

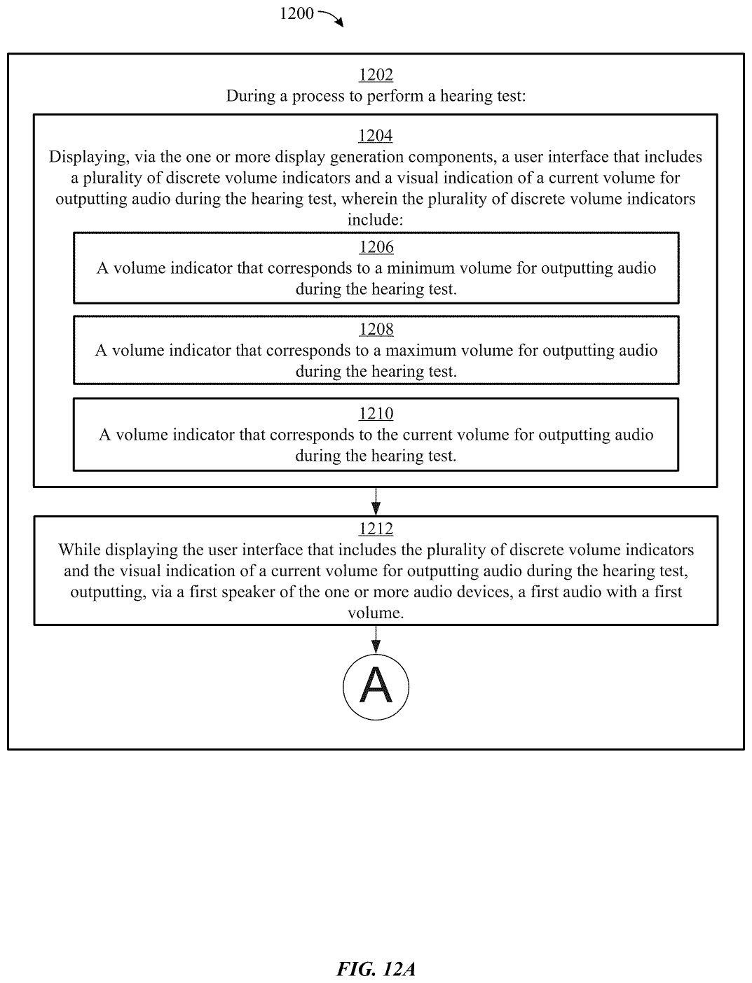

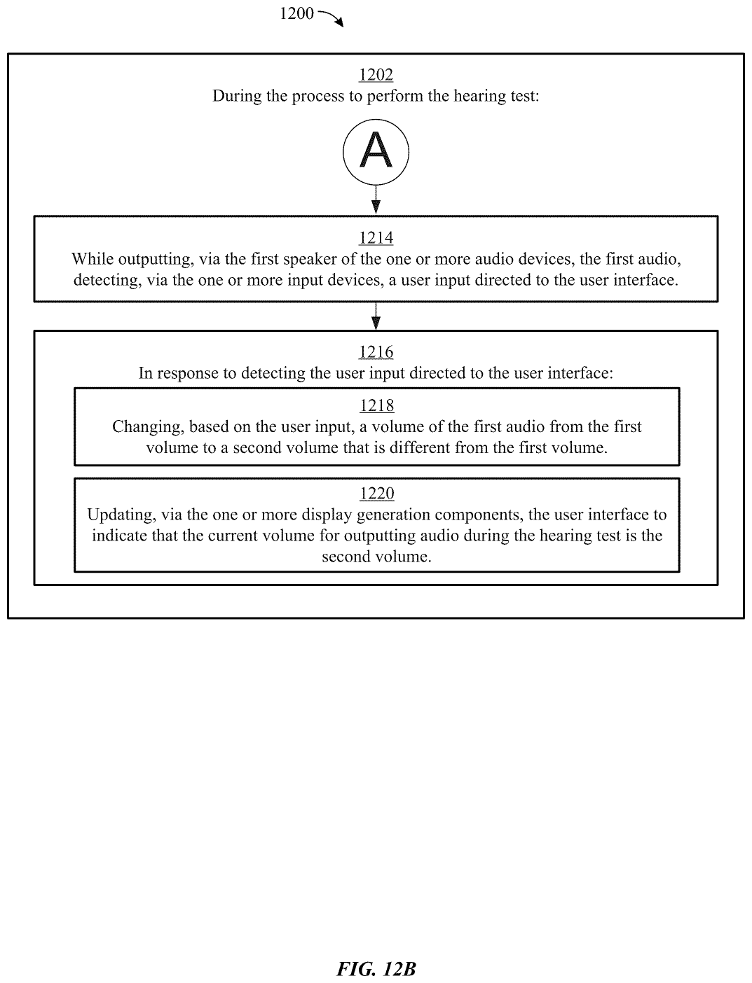

Below, FIGS. 1A-1B, 2, 3A, 4A-4B, and 5A-5B provide a description of exemplary devices for performing the techniques for managing event notifications. FIGS. 6A-6BH illustrate exemplary user interfaces for managing hearing health. FIG. 7 is a flow diagram illustrating methods of prompting a user to initiate a hearing test, in some embodiments. FIG. 8 is a flow diagram illustrating methods of modifying audio characteristics of different types of audio, in some embodiments. The user interfaces in FIGS. 6A-6BH are used to illustrate the processes described below, including the processes in FIGS. 7-8. FIGS. 9A-9D illustrate example user interfaces for displaying a visual indication of a current level of environmental noise, in accordance with some embodiments. FIG. 10 is a flow diagram illustrating methods of displaying a visual indication of a current level of environmental noise, in accordance with some embodiments. The user interfaces in FIGS. 6A-6BH and 9A-9D are used to illustrate the processes described below, including the processes in FIG. 10. FIGS. 11A-11N illustrate example user interfaces for performing hearing tests, in accordance with some embodiments. FIGS. 12A-12B are a flow diagram illustrating methods of performing hearing tests, in accordance with some embodiments. The user interfaces in FIGS. 11A-11N are used to illustrate the processes described below, including the processes in FIGS. 12A-12B.

The processes described below enhance the operability of the devices and make the user-device interfaces more efficient (e.g., by helping the user to provide proper inputs and reducing user mistakes when operating/interacting with the device) through various techniques, including by providing improved visual feedback to the user, reducing the number of inputs needed to perform an operation, providing additional control options without cluttering the user interface with additional displayed controls, performing an operation when a set of conditions has been met without requiring further user input, and/or additional techniques. These techniques also reduce power usage and improve battery life of the device by enabling the user to use the device more quickly and efficiently.

In addition, in methods described herein where one or more steps are contingent upon one or more conditions having been met, it should be understood that the described method can be repeated in multiple repetitions so that over the course of the repetitions all of the conditions upon which steps in the method are contingent have been met in different repetitions of the method. For example, if a method requires performing a first step if a condition is satisfied, and a second step if the condition is not satisfied, then a person of ordinary skill would appreciate that the claimed steps are repeated until the condition has been both satisfied and not satisfied, in no particular order. Thus, a method described with one or more steps that are contingent upon one or more conditions having been met could be rewritten as a method that is repeated until each of the conditions described in the method has been met. This, however, is not required of system or computer readable medium claims where the system or computer readable medium contains instructions for performing the contingent operations based on the satisfaction of the corresponding one or more conditions and thus is capable of determining whether the contingency has or has not been satisfied without explicitly repeating steps of a method until all of the conditions upon which steps in the method are contingent have been met. A person having ordinary skill in the art would also understand that, similar to a method with contingent steps, a system or computer readable storage medium can repeat the steps of a method as many times as are needed to ensure that all of the contingent steps have been performed.

Although the following description uses terms “first,” “second,” etc. to describe various elements, these elements should not be limited by the terms. In some embodiments, these terms are used to distinguish one element from another. For example, a first touch could be termed a second touch, and, similarly, a second touch could be termed a first touch, without departing from the scope of the various described embodiments. In some embodiments, the first touch and the second touch are two separate references to the same touch. In some embodiments, the first touch and the second touch are both touches, but they are not the same touch.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Embodiments of electronic devices, user interfaces for such devices, and associated processes for using such devices are described. In some embodiments, the device is a portable communications device, such as a mobile telephone, that also contains other functions, such as PDA and/or music player functions. Exemplary embodiments of portable multifunction devices include, without limitation, the iPhone®, iPod Touch®, and iPad® devices from Apple Inc. of Cupertino, California. Other portable electronic devices, such as laptops or tablet computers with touch-sensitive surfaces (e.g., touch screen displays and/or touchpads), are, optionally, used. It should also be understood that, in some embodiments, the device is not a portable communications device, but is a desktop computer with a touch-sensitive surface (e.g., a touch screen display and/or a touchpad). In some embodiments, the electronic device is a computer system that is in communication (e.g., via wireless communication, via wired communication) with a display generation component. The display generation component is configured to provide visual output, such as display via a CRT display, display via an LED display, or display via image projection. In some embodiments, the display generation component is integrated with the computer system. In some embodiments, the display generation component is separate from the computer system. As used herein, “displaying” content includes causing to display the content (e.g., video data rendered or decoded by display controller 156) by transmitting, via a wired or wireless connection, data (e.g., image data or video data) to an integrated or external display generation component to visually produce the content.

In the discussion that follows, an electronic device that includes a display and a touch-sensitive surface is described. It should be understood, however, that the electronic device optionally includes one or more other physical user-interface devices, such as a physical keyboard, a mouse, and/or a joystick.

The device typically supports a variety of applications, such as one or more of the following: a drawing application, a presentation application, a word processing application, a website creation application, a disk authoring application, a spreadsheet application, a gaming application, a telephone application, a video conferencing application, an e-mail application, an instant messaging application, a workout support application, a photo management application, a digital camera application, a digital video camera application, a web browsing application, a digital music player application, and/or a digital video player application.

The various applications that are executed on the device optionally use at least one common physical user-interface device, such as the touch-sensitive surface. One or more functions of the touch-sensitive surface as well as corresponding information displayed on the device are, optionally, adjusted and/or varied from one application to the next and/or within a respective application. In this way, a common physical architecture (such as the touch-sensitive surface) of the device optionally supports the variety of applications with user interfaces that are intuitive and transparent to the user.

Attention is now directed toward embodiments of portable devices with touch-sensitive displays. FIG. 1A is a block diagram illustrating portable multifunction device 100 with touch-sensitive display system 112 in accordance with some embodiments. Touch-sensitive display 112 is sometimes called a “touch screen” for convenience and is sometimes known as or called a “touch-sensitive display system.” Device 100 includes memory 102 (which optionally includes one or more computer-readable storage mediums), memory controller 122, one or more processing units (CPUs) 120, peripherals interface 118, RF circuitry 108, audio circuitry 110, speaker 111, microphone 113, input/output (I/O) subsystem 106, other input control devices 116, and external port 124. Device 100 optionally includes one or more optical sensors 164. Device 100 optionally includes one or more contact intensity sensors 165 for detecting intensity of contacts on device 100 (e.g., a touch-sensitive surface such as touch-sensitive display system 112 of device 100). Device 100 optionally includes one or more tactile output generators 167 for generating tactile outputs on device 100 (e.g., generating tactile outputs on a touch-sensitive surface such as touch-sensitive display system 112 of device 100 or touchpad 355 of device 300). These components optionally communicate over one or more communication buses or signal lines 103.

As used in the specification and claims, the term “intensity” of a contact on a touch-sensitive surface refers to the force or pressure (force per unit area) of a contact (e.g., a finger contact) on the touch-sensitive surface, or to a substitute (proxy) for the force or pressure of a contact on the touch-sensitive surface. The intensity of a contact has a range of values that includes at least four distinct values and more typically includes hundreds of distinct values (e.g., at least 256). Intensity of a contact is, optionally, determined (or measured) using various approaches and various sensors or combinations of sensors. For example, one or more force sensors underneath or adjacent to the touch-sensitive surface are, optionally, used to measure force at various points on the touch-sensitive surface. In some implementations, force measurements from multiple force sensors are combined (e.g., a weighted average) to determine an estimated force of a contact. Similarly, a pressure-sensitive tip of a stylus is, optionally, used to determine a pressure of the stylus on the touch-sensitive surface. Alternatively, the size of the contact area detected on the touch-sensitive surface and/or changes thereto, the capacitance of the touch-sensitive surface proximate to the contact and/or changes thereto, and/or the resistance of the touch-sensitive surface proximate to the contact and/or changes thereto are, optionally, used as a substitute for the force or pressure of the contact on the touch-sensitive surface. In some implementations, the substitute measurements for contact force or pressure are used directly to determine whether an intensity threshold has been exceeded (e.g., the intensity threshold is described in units corresponding to the substitute measurements). In some implementations, the substitute measurements for contact force or pressure are converted to an estimated force or pressure, and the estimated force or pressure is used to determine whether an intensity threshold has been exceeded (e.g., the intensity threshold is a pressure threshold measured in units of pressure). Using the intensity of a contact as an attribute of a user input allows for user access to additional device functionality that may otherwise not be accessible by the user on a reduced-size device with limited real estate for displaying affordances (e.g., on a touch-sensitive display) and/or receiving user input (e.g., via a touch-sensitive display, a touch-sensitive surface, or a physical/mechanical control such as a knob or a button).

As used in the specification and claims, the term “tactile output” refers to physical displacement of a device relative to a previous position of the device, physical displacement of a component (e.g., a touch-sensitive surface) of a device relative to another component (e.g., housing) of the device, or displacement of the component relative to a center of mass of the device that will be detected by a user with the user's sense of touch. For example, in situations where the device or the component of the device is in contact with a surface of a user that is sensitive to touch (e.g., a finger, palm, or other part of a user's hand), the tactile output generated by the physical displacement will be interpreted by the user as a tactile sensation corresponding to a perceived change in physical characteristics of the device or the component of the device. For example, movement of a touch-sensitive surface (e.g., a touch-sensitive display or trackpad) is, optionally, interpreted by the user as a “down click” or “up click” of a physical actuator button. In some cases, a user will feel a tactile sensation such as an “down click” or “up click” even when there is no movement of a physical actuator button associated with the touch-sensitive surface that is physically pressed (e.g., displaced) by the user's movements. As another example, movement of the touch-sensitive surface is, optionally, interpreted or sensed by the user as “roughness” of the touch-sensitive surface, even when there is no change in smoothness of the touch-sensitive surface. While such interpretations of touch by a user will be subject to the individualized sensory perceptions of the user, there are many sensory perceptions of touch that are common to a large majority of users. Thus, when a tactile output is described as corresponding to a particular sensory perception of a user (e.g., an “up click,” a “down click,” “roughness”), unless otherwise stated, the generated tactile output corresponds to physical displacement of the device or a component thereof that will generate the described sensory perception for a typical (or average) user.

It should be appreciated that device 100 is only one example of a portable multifunction device, and that device 100 optionally has more or fewer components than shown, optionally combines two or more components, or optionally has a different configuration or arrangement of the components. The various components shown in FIG. 1A are implemented in hardware, software, or a combination of both hardware and software, including one or more signal processing and/or application-specific integrated circuits.

Memory 102 optionally includes high-speed random access memory and optionally also includes non-volatile memory, such as one or more magnetic disk storage devices, flash memory devices, or other non-volatile solid-state memory devices. Memory controller 122 optionally controls access to memory 102 by other components of device 100.

Peripherals interface 118 can be used to couple input and output peripherals of the device to CPU 120 and memory 102. The one or more processors 120 run or execute various software programs (such as computer programs (e.g., including instructions)) and/or sets of instructions stored in memory 102 to perform various functions for device 100 and to process data. In some embodiments, peripherals interface 118, CPU 120, and memory controller 122 are, optionally, implemented on a single chip, such as chip 104. In some other embodiments, they are, optionally, implemented on separate chips.

RF (radio frequency) circuitry 108 receives and sends RF signals, also called electromagnetic signals. RF circuitry 108 converts electrical signals to/from electromagnetic signals and communicates with communications networks and other communications devices via the electromagnetic signals. RF circuitry 108 optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry 108 optionally communicates with networks, such as the Internet, also referred to as the World Wide Web (WWW), an intranet and/or a wireless network, such as a cellular telephone network, a wireless local area network (LAN) and/or a metropolitan area network (MAN), and other devices by wireless communication. The RF circuitry 108 optionally includes well-known circuitry for detecting near field communication (NFC) fields, such as by a short-range communication radio. The wireless communication optionally uses any of a plurality of communications standards, protocols, and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Bluetooth Low Energy (BTLE), Wireless Fidelity (Wi-Fi) (e.g., IEEE 802.11a, IEEE 802.11b, IEEE 802.11g, IEEE 802.11n, and/or IEEE 802.11ac), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

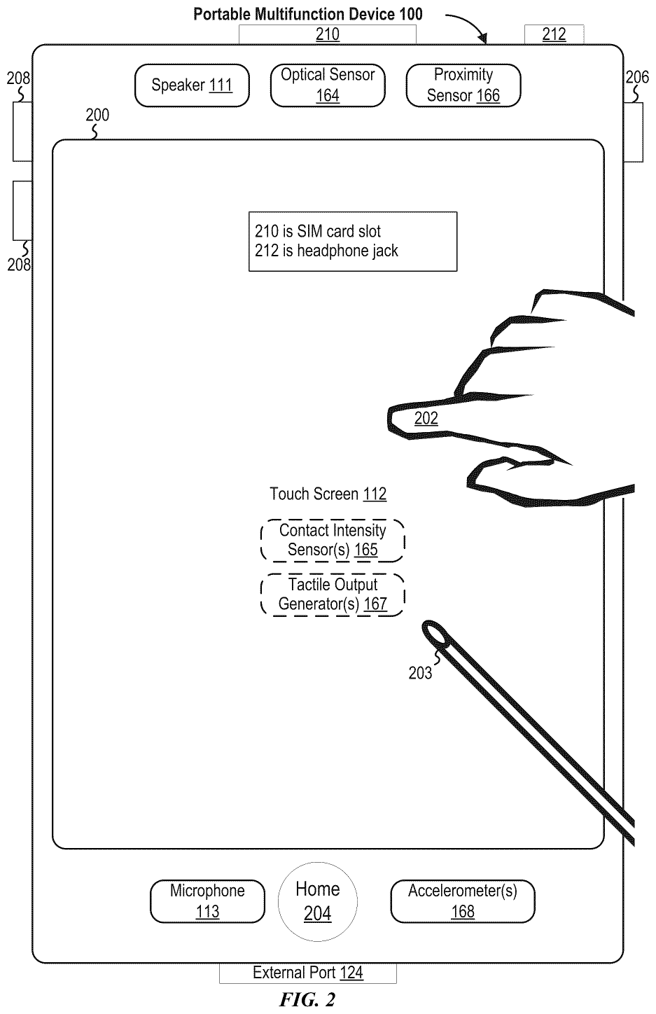

Audio circuitry 110, speaker 111, and microphone 113 provide an audio interface between a user and device 100. Audio circuitry 110 receives audio data from peripherals interface 118, converts the audio data to an electrical signal, and transmits the electrical signal to speaker 111. Speaker 111 converts the electrical signal to human-audible sound waves. Audio circuitry 110 also receives electrical signals converted by microphone 113 from sound waves. Audio circuitry 110 converts the electrical signal to audio data and transmits the audio data to peripherals interface 118 for processing. Audio data is, optionally, retrieved from and/or transmitted to memory 102 and/or RF circuitry 108 by peripherals interface 118. In some embodiments, audio circuitry 110 also includes a headset jack (e.g., 212, FIG. 2). The headset jack provides an interface between audio circuitry 110 and removable audio input/output peripherals, such as output-only headphones or a headset with both output (e.g., a headphone for one or both ears) and input (e.g., a microphone).

I/O subsystem 106 couples input/output peripherals on device 100, such as touch screen 112 and other input control devices 116, to peripherals interface 118. I/O subsystem 106 optionally includes display controller 156, optical sensor controller 158, depth camera controller 169, intensity sensor controller 159, haptic feedback controller 161, and one or more input controllers 160 for other input or control devices. The one or more input controllers 160 receive/send electrical signals from/to other input control devices 116. The other input control devices 116 optionally include physical buttons (e.g., push buttons, rocker buttons, etc.), dials, slider switches, joysticks, click wheels, and so forth. In some embodiments, input controller(s) 160 are, optionally, coupled to any (or none) of the following: a keyboard, an infrared port, a USB port, and a pointer device such as a mouse. The one or more buttons (e.g., 208, FIG. 2) optionally include an up/down button for volume control of speaker 111 and/or microphone 113. The one or more buttons optionally include a push button (e.g., 206, FIG. 2). In some embodiments, the electronic device is a computer system that is in communication (e.g., via wireless communication, via wired communication) with one or more input devices. In some embodiments, the one or more input devices include a touch-sensitive surface (e.g., a trackpad, as part of a touch-sensitive display). In some embodiments, the one or more input devices include one or more camera sensors (e.g., one or more optical sensors 164 and/or one or more depth camera sensors 175), such as for tracking a user's gestures (e.g., hand gestures and/or air gestures) as input. In some embodiments, the one or more input devices are integrated with the computer system. In some embodiments, the one or more input devices are separate from the computer system. In some embodiments, an air gesture is a gesture that is detected without the user touching an input element that is part of the device (or independently of an input element that is a part of the device) and is based on detected motion of a portion of the user's body through the air including motion of the user's body relative to an absolute reference (e.g., an angle of the user's arm relative to the ground or a distance of the user's hand relative to the ground), relative to another portion of the user's body (e.g., movement of a hand of the user relative to a shoulder of the user, movement of one hand of the user relative to another hand of the user, and/or movement of a finger of the user relative to another finger or portion of a hand of the user), and/or absolute motion of a portion of the user's body (e.g., a tap gesture that includes movement of a hand in a predetermined pose by a predetermined amount and/or speed, or a shake gesture that includes a predetermined speed or amount of rotation of a portion of the user's body).

A quick press of the push button optionally disengages a lock of touch screen 112 or optionally begins a process that uses gestures on the touch screen to unlock the device, as described in U.S. patent application Ser. No. 11/322,549, “Unlocking a Device by Performing Gestures on an Unlock Image,” filed Dec. 23, 2005, U.S. Pat. No. 7,657,849, which is hereby incorporated by reference in its entirety. A longer press of the push button (e.g., 206) optionally turns power to device 100 on or off. The functionality of one or more of the buttons are, optionally, user-customizable. Touch screen 112 is used to implement virtual or soft buttons and one or more soft keyboards.

Touch-sensitive display 112 provides an input interface and an output interface between the device and a user. Display controller 156 receives and/or sends electrical signals from/to touch screen 112. Touch screen 112 displays visual output to the user. The visual output optionally includes graphics, text, icons, video, and any combination thereof (collectively termed “graphics”). In some embodiments, some or all of the visual output optionally corresponds to user-interface objects.

Touch screen 112 has a touch-sensitive surface, sensor, or set of sensors that accepts input from the user based on haptic and/or tactile contact. Touch screen 112 and display controller 156 (along with any associated modules and/or sets of instructions in memory 102) detect contact (and any movement or breaking of the contact) on touch screen 112 and convert the detected contact into interaction with user-interface objects (e.g., one or more soft keys, icons, web pages, or images) that are displayed on touch screen 112. In an exemplary embodiment, a point of contact between touch screen 112 and the user corresponds to a finger of the user.

Touch screen 112 optionally uses LCD (liquid crystal display) technology, LPD (light emitting polymer display) technology, or LED (light emitting diode) technology, although other display technologies are used in other embodiments. Touch screen 112 and display controller 156 optionally detect contact and any movement or breaking thereof using any of a plurality of touch sensing technologies now known or later developed, including but not limited to capacitive, resistive, infrared, and surface acoustic wave technologies, as well as other proximity sensor arrays or other elements for determining one or more points of contact with touch screen 112. In an exemplary embodiment, projected mutual capacitance sensing technology is used, such as that found in the iPhone® and iPod Touch® from Apple Inc. of Cupertino, California.

A touch-sensitive display in some embodiments of touch screen 112 is, optionally, analogous to the multi-touch sensitive touchpads described in the following U.S. Pat. No. 6,323,846 (Westerman et al.), 6,570,557 (Westerman et al.), and/or 6,677,932 (Westerman), and/or U.S. Patent Publication 2002/0015024A1, each of which is hereby incorporated by reference in its entirety. However, touch screen 112 displays visual output from device 100, whereas touch-sensitive touchpads do not provide visual output.

A touch-sensitive display in some embodiments of touch screen 112 is described in the following applications: (1) U.S. patent application Ser. No. 11/381,313, “Multipoint Touch Surface Controller,” filed May 2, 2006; (2) U.S. patent application Ser. No. 10/840,862, “Multipoint Touchscreen,” filed May 6, 2004; (3) U.S. patent application Ser. No. 10/903,964, “Gestures For Touch Sensitive Input Devices,” filed Jul. 30, 2004; (4) U.S. patent application Ser. No. 11/048,264, “Gestures For Touch Sensitive Input Devices,” filed Jan. 31, 2005; (5) U.S. patent application Ser. No. 11/038,590, “Mode-Based Graphical User Interfaces For Touch Sensitive Input Devices,” filed Jan. 18, 2005; (6) U.S. patent application Ser. No. 11/228,758, “Virtual Input Device Placement On A Touch Screen User Interface,” filed Sep. 16, 2005; (7) U.S. patent application Ser. No. 11/228,700, “Operation Of A Computer With A Touch Screen Interface,” filed Sep. 16, 2005; (8) U.S. patent application Ser. No. 11/228,737, “Activating Virtual Keys Of A Touch-Screen Virtual Keyboard,” filed Sep. 16, 2005; and (9) U.S. patent application Ser. No. 11/367,749, “Multi-Functional Hand-Held Device,” filed Mar. 3, 2006. All of these applications are incorporated by reference herein in their entirety.

Touch screen 112 optionally has a video resolution in excess of 100 dpi. In some embodiments, the touch screen has a video resolution of approximately 160 dpi. The user optionally makes contact with touch screen 112 using any suitable object or appendage, such as a stylus, a finger, and so forth. In some embodiments, the user interface is designed to work primarily with finger-based contacts and gestures, which can be less precise than stylus-based input due to the larger area of contact of a finger on the touch screen. In some embodiments, the device translates the rough finger-based input into a precise pointer/cursor position or command for performing the actions desired by the user.

In some embodiments, in addition to the touch screen, device 100 optionally includes a touchpad for activating or deactivating particular functions. In some embodiments, the touchpad is a touch-sensitive area of the device that, unlike the touch screen, does not display visual output. The touchpad is, optionally, a touch-sensitive surface that is separate from touch screen 112 or an extension of the touch-sensitive surface formed by the touch screen.

Device 100 also includes power system 162 for powering the various components. Power system 162 optionally includes a power management system, one or more power sources (e.g., battery, alternating current (AC)), a recharging system, a power failure detection circuit, a power converter or inverter, a power status indicator (e.g., a light-emitting diode (LED)) and any other components associated with the generation, management and distribution of power in portable devices.

Device 100 optionally also includes secure element 163 for securely storing information. In some embodiments, secure element 163 is a hardware component (e.g., a secure microcontroller chip) configured to securely store data or an algorithm. In some embodiments, secure element 163 provides (e.g., releases) secure information (e.g., payment information (e.g., an account number and/or a transaction-specific dynamic security code), identification information (e.g., credentials of a state-approved digital identification), and/or authentication information (e.g., data generated using a cryptography engine and/or by performing asymmetric cryptography operations)). In some embodiments, secure element 163 provides (or releases) the secure information in response to device 100 receiving authorization, such as a user authentication (e.g., fingerprint authentication; passcode authentication; detecting double-press of a hardware button when device 100 is in an unlocked state, and optionally, while device 100 has been continuously on a user's wrist since device 100 was unlocked by providing authentication credentials to device 100, where the continuous presence of device 100 on the user's wrist is determined by periodically checking that the device is in contact with the user's skin). For example, device 100 detects a fingerprint at a fingerprint sensor (e.g., a fingerprint sensor integrated into a button) of device 100. Device 100 determines whether the detected fingerprint is consistent with an enrolled fingerprint. In accordance with a determination that the fingerprint is consistent with the enrolled fingerprint, secure element 163 provides (e.g., releases) the secure information. In accordance with a determination that the fingerprint is not consistent with the enrolled fingerprint, secure element 163 forgoes providing (e.g., releasing) the secure information.

Device 100 optionally also includes one or more optical sensors 164. FIG. 1A shows an optical sensor coupled to optical sensor controller 158 in I/O subsystem 106. Optical sensor 164 optionally includes charge-coupled device (CCD) or complementary metal-oxide semiconductor (CMOS) phototransistors. Optical sensor 164 receives light from the environment, projected through one or more lenses, and converts the light to data representing an image. In conjunction with imaging module 143 (also called a camera module), optical sensor 164 optionally captures still images or video. In some embodiments, an optical sensor is located on the back of device 100, opposite touch screen display 112 on the front of the device so that the touch screen display is enabled for use as a viewfinder for still and/or video image acquisition. In some embodiments, an optical sensor is located on the front of the device so that the user's image is, optionally, obtained for video conferencing while the user views the other video conference participants on the touch screen display. In some embodiments, the position of optical sensor 164 can be changed by the user (e.g., by rotating the lens and the sensor in the device housing) so that a single optical sensor 164 is used along with the touch screen display for both video conferencing and still and/or video image acquisition.

Device 100 optionally also includes one or more depth camera sensors 175. FIG. 1A shows a depth camera sensor coupled to depth camera controller 169 in I/O subsystem 106. Depth camera sensor 175 receives data from the environment to create a three dimensional model of an object (e.g., a face) within a scene from a viewpoint (e.g., a depth camera sensor). In some embodiments, in conjunction with imaging module 143 (also called a camera module), depth camera sensor 175 is optionally used to determine a depth map of different portions of an image captured by the imaging module 143. In some embodiments, a depth camera sensor is located on the front of device 100 so that the user's image with depth information is, optionally, obtained for video conferencing while the user views the other video conference participants on the touch screen display and to capture selfies with depth map data. In some embodiments, the depth camera sensor 175 is located on the back of device, or on the back and the front of the device 100. In some embodiments, the position of depth camera sensor 175 can be changed by the user (e.g., by rotating the lens and the sensor in the device housing) so that a depth camera sensor 175 is used along with the touch screen display for both video conferencing and still and/or video image acquisition.

Device 100 optionally also includes one or more contact intensity sensors 165. FIG. 1A shows a contact intensity sensor coupled to intensity sensor controller 159 in I/O subsystem 106. Contact intensity sensor 165 optionally includes one or more piezoresistive strain gauges, capacitive force sensors, electric force sensors, piezoelectric force sensors, optical force sensors, capacitive touch-sensitive surfaces, or other intensity sensors (e.g., sensors used to measure the force (or pressure) of a contact on a touch-sensitive surface). Contact intensity sensor 165 receives contact intensity information (e.g., pressure information or a proxy for pressure information) from the environment. In some embodiments, at least one contact intensity sensor is collocated with, or proximate to, a touch-sensitive surface (e.g., touch-sensitive display system 112). In some embodiments, at least one contact intensity sensor is located on the back of device 100, opposite touch screen display 112, which is located on the front of device 100.

Device 100 optionally also includes one or more proximity sensors 166. FIG. 1A shows proximity sensor 166 coupled to peripherals interface 118. Alternately, proximity sensor 166 is, optionally, coupled to input controller 160 in I/O subsystem 106. Proximity sensor 166 optionally performs as described in U.S. patent application Ser. No. 11/241,839, “Proximity Detector In Handheld Device”; Ser. No. 11/240,788, “Proximity Detector In Handheld Device”; Ser. No. 11/620,702, “Using Ambient Light Sensor To Augment Proximity Sensor Output”; Ser. No. 11/586,862, “Automated Response To And Sensing Of User Activity In Portable Devices”; and Ser. No. 11/638,251, “Methods And Systems For Automatic Configuration Of Peripherals,” which are hereby incorporated by reference in their entirety. In some embodiments, the proximity sensor turns off and disables touch screen 112 when the multifunction device is placed near the user's ear (e.g., when the user is making a phone call).

Device 100 optionally also includes one or more tactile output generators 167. FIG. 1A shows a tactile output generator coupled to haptic feedback controller 161 in I/O subsystem 106. Tactile output generator 167 optionally includes one or more electroacoustic devices such as speakers or other audio components and/or electromechanical devices that convert energy into linear motion such as a motor, solenoid, electroactive polymer, piezoelectric actuator, electrostatic actuator, or other tactile output generating component (e.g., a component that converts electrical signals into tactile outputs on the device). Contact intensity sensor 165 receives tactile feedback generation instructions from haptic feedback module 133 and generates tactile outputs on device 100 that are capable of being sensed by a user of device 100. In some embodiments, at least one tactile output generator is collocated with, or proximate to, a touch-sensitive surface (e.g., touch-sensitive display system 112) and, optionally, generates a tactile output by moving the touch-sensitive surface vertically (e.g., in/out of a surface of device 100) or laterally (e.g., back and forth in the same plane as a surface of device 100). In some embodiments, at least one tactile output generator sensor is located on the back of device 100, opposite touch screen display 112, which is located on the front of device 100.

Device 100 optionally also includes one or more accelerometers 168. FIG. 1A shows accelerometer 168 coupled to peripherals interface 118. Alternately, accelerometer 168 is, optionally, coupled to an input controller 160 in I/O subsystem 106. Accelerometer 168 optionally performs as described in U.S. Patent Publication No. 20050190059, “Acceleration-based Theft Detection System for Portable Electronic Devices,” and U.S. Patent Publication No. 20060017692, “Methods And Apparatuses For Operating A Portable Device Based On An Accelerometer,” both of which are incorporated by reference herein in their entirety. In some embodiments, information is displayed on the touch screen display in a portrait view or a landscape view based on an analysis of data received from the one or more accelerometers. Device 100 optionally includes, in addition to accelerometer(s) 168, a magnetometer and a GPS (or GLONASS or other global navigation system) receiver for obtaining information concerning the location and orientation (e.g., portrait or landscape) of device 100.

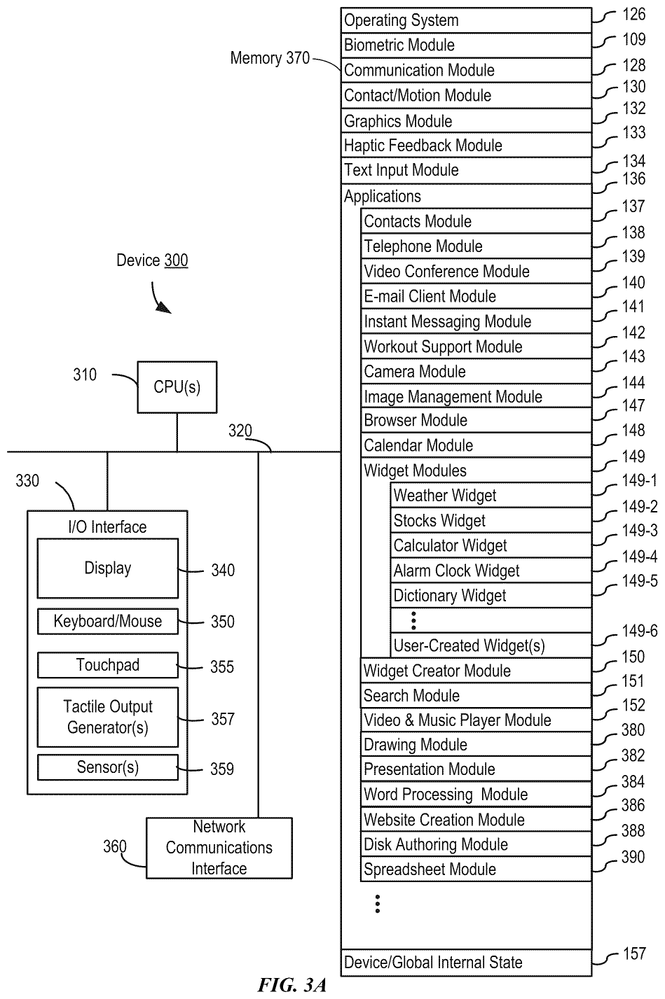

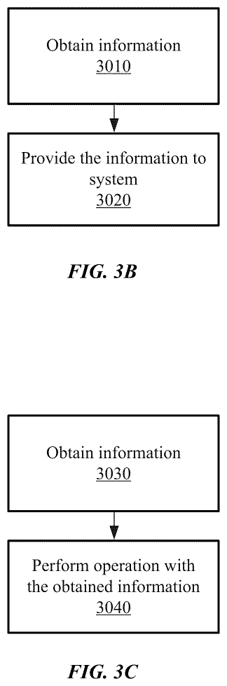

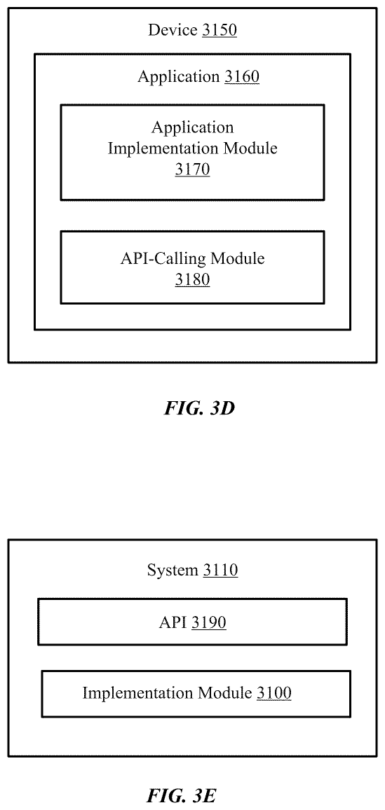

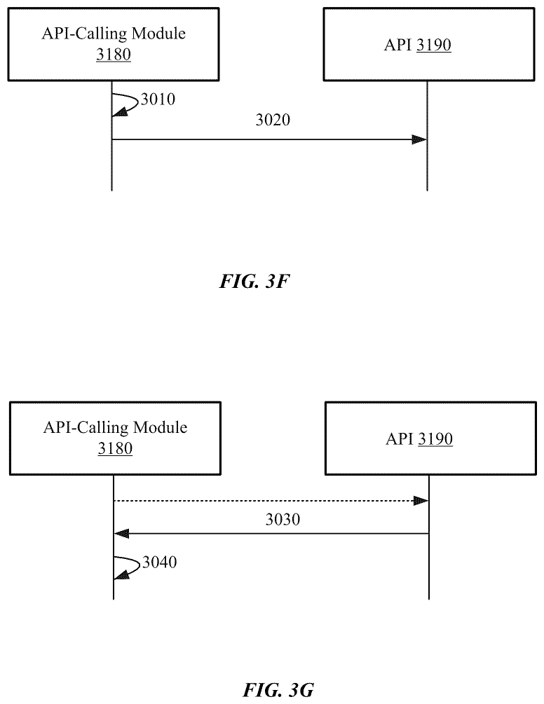

In some embodiments, the software components stored in memory 102 include operating system 126, biometric module 109, communication module (or set of instructions) 128, contact/motion module (or set of instructions) 130, graphics module (or set of instructions) 132, text input module (or set of instructions) 134, Global Positioning System (GPS) module (or set of instructions) 135, authentication module 105, and applications (or sets of instructions) 136. Furthermore, in some embodiments, memory 102 (FIG. 1A) or 370 (FIG. 3A) stores device/global internal state 157, as shown in FIGS. 1A and 3A. Device/global internal state 157 includes one or more of: active application state, indicating which applications, if any, are currently active; display state, indicating what applications, views or other information occupy various regions of touch screen display 112; sensor state, including information obtained from the device's various sensors and input control devices 116; and location information concerning the device's location and/or attitude.

Operating system 126 (e.g., Darwin, RTXC, LINUX, UNIX, OS X, IOS, WINDOWS, or an embedded operating system such as VxWorks) includes various software components and/or drivers for controlling and managing general system tasks (e.g., memory management, storage device control, power management, etc.) and facilitates communication between various hardware and software components.

Communication module 128 facilitates communication with other devices over one or more external ports 124 and also includes various software components for handling data received by RF circuitry 108 and/or external port 124. External port 124 (e.g., Universal Serial Bus (USB), FIREWIRE, etc.) is adapted for coupling directly to other devices or indirectly over a network (e.g., the Internet, wireless LAN, etc.). In some embodiments, the external port is a multi-pin (e.g., 30-pin) connector that is the same as, or similar to and/or compatible with, the 30-pin connector used on iPod® (trademark of Apple Inc.) devices.

Biometric module 109 optionally stores information about one or more enrolled biometric features (e.g., fingerprint feature information, facial recognition feature information, eye and/or iris feature information) for use to verify whether received biometric information matches the enrolled biometric features. In some embodiments, the information stored about the one or more enrolled biometric features includes data that enables the comparison between the stored information and received biometric information without including enough information to reproduce the enrolled biometric features. In some embodiments, biometric module 109 stores the information about the enrolled biometric features in association with a user account of device 100. In some embodiments, biometric module 109 compares the received biometric information to an enrolled biometric feature to determine whether the received biometric information matches the enrolled biometric feature.

Contact/motion module 130 optionally detects contact with touch screen 112 (in conjunction with display controller 156) and other touch-sensitive devices (e.g., a touchpad or physical click wheel). Contact/motion module 130 includes various software components for performing various operations related to detection of contact, such as determining if contact has occurred (e.g., detecting a finger-down event), determining an intensity of the contact (e.g., the force or pressure of the contact or a substitute for the force or pressure of the contact), determining if there is movement of the contact and tracking the movement across the touch-sensitive surface (e.g., detecting one or more finger-dragging events), and determining if the contact has ceased (e.g., detecting a finger-up event or a break in contact). Contact/motion module 130 receives contact data from the touch-sensitive surface. Determining movement of the point of contact, which is represented by a series of contact data, optionally includes determining speed (magnitude), velocity (magnitude and direction), and/or an acceleration (a change in magnitude and/or direction) of the point of contact. These operations are, optionally, applied to single contacts (e.g., one finger contacts) or to multiple simultaneous contacts (e.g., “multitouch”/multiple finger contacts). In some embodiments, contact/motion module 130 and display controller 156 detect contact on a touchpad.

In some embodiments, contact/motion module 130 uses a set of one or more intensity thresholds to determine whether an operation has been performed by a user (e.g., to determine whether a user has “clicked” on an icon). In some embodiments, at least a subset of the intensity thresholds are determined in accordance with software parameters (e.g., the intensity thresholds are not determined by the activation thresholds of particular physical actuators and can be adjusted without changing the physical hardware of device 100). For example, a mouse “click” threshold of a trackpad or touch screen display can be set to any of a large range of predefined threshold values without changing the trackpad or touch screen display hardware. Additionally, in some implementations, a user of the device is provided with software settings for adjusting one or more of the set of intensity thresholds (e.g., by adjusting individual intensity thresholds and/or by adjusting a plurality of intensity thresholds at once with a system-level click “intensity” parameter).

Contact/motion module 130 optionally detects a gesture input by a user. Different gestures on the touch-sensitive surface have different contact patterns (e.g., different motions, timings, and/or intensities of detected contacts). Thus, a gesture is, optionally, detected by detecting a particular contact pattern. For example, detecting a finger tap gesture includes detecting a finger-down event followed by detecting a finger-up (liftoff) event at the same position (or substantially the same position) as the finger-down event (e.g., at the position of an icon). As another example, detecting a finger swipe gesture on the touch-sensitive surface includes detecting a finger-down event followed by detecting one or more finger-dragging events, and subsequently followed by detecting a finger-up (liftoff) event.

Graphics module 132 includes various known software components for rendering and displaying graphics on touch screen 112 or other display, including components for changing the visual impact (e.g., brightness, transparency, saturation, contrast, or other visual property) of graphics that are displayed. As used herein, the term “graphics” includes any object that can be displayed to a user, including, without limitation, text, web pages, icons (such as user-interface objects including soft keys), digital images, videos, animations, and the like.

In some embodiments, graphics module 132 stores data representing graphics to be used. Each graphic is, optionally, assigned a corresponding code. Graphics module 132 receives, from applications etc., one or more codes specifying graphics to be displayed along with, if necessary, coordinate data and other graphic property data, and then generates screen image data to output to display controller 156.

Haptic feedback module 133 includes various software components for generating instructions used by tactile output generator(s) 167 to produce tactile outputs at one or more locations on device 100 in response to user interactions with device 100.

Text input module 134, which is, optionally, a component of graphics module 132, provides soft keyboards for entering text in various applications (e.g., contacts module 137, e-mail client module 140, IM module 141, browser module 147, and any other application that needs text input).

GPS module 135 determines the location of the device and provides this information for use in various applications (e.g., to telephone module 138 for use in location-based dialing; to camera module 143 as picture/video metadata; and to applications that provide location-based services such as weather widgets, local yellow page widgets, and map/navigation widgets).

Authentication module 105 determines whether a requested operation (e.g., requested by an application of applications 136) is authorized to be performed. In some embodiments, authentication module 105 receives for an operation to be perform that optionally requires authentication. Authentication module 105 determines whether the operation is authorized to be performed, such as based on a series of factors, including the lock status of device 100, the location of device 100, whether a security delay has elapsed, whether received biometric information matches enrolled biometric features, and/or other factors. Once authentication module 105 determines that the operation is authorized to be performed, authentication module 105 triggers performance of the operation.

Applications 136 optionally include the following modules (or sets of instructions), or a subset or superset thereof:
Contacts module 137 (sometimes called an address book or contact list);
Telephone module 138;
Video conference module 139;
E-mail client module 140;
Instant messaging (IM) module 141;
Workout support module 142;
Camera module 143 for still and/or video images;
Image management module 144;
Video player module;
Music player module;
Browser module 147;
Calendar module 148;
Widget modules 149, which optionally include one or more of: weather widget 149-1, stocks widget 149-2, calculator widget 149-3, alarm clock widget 149-4, dictionary widget 149-5, and other widgets obtained by the user, as well as user-created widgets 149-6;
Widget creator module 150 for making user-created widgets 149-6;
Search module 151;
Video and music player module 152, which merges video player module and music player module;
Notes module 153;
Map module 154; and/or
Online video module 155.

Examples of other applications 136 that are, optionally, stored in memory 102 include other word processing applications, other image editing applications, drawing applications, presentation applications, JAVA-enabled applications, encryption, digital rights management, voice recognition, and voice replication.

In conjunction with touch screen 112, display controller 156, contact/motion module 130, graphics module 132, and text input module 134, contacts module 137 are, optionally, used to manage an address book or contact list (e.g., stored in application internal state 192 of contacts module 137 in memory 102 or memory 370), including: adding name(s) to the address book; deleting name(s) from the address book; associating telephone number(s), e-mail address(es), physical address(es) or other information with a name; associating an image with a name; categorizing and sorting names; providing telephone numbers or e-mail addresses to initiate and/or facilitate communications by telephone module 138, video conference module 139, e-mail client module 140, or IM module 141; and so forth.

In conjunction with RF circuitry 108, audio circuitry 110, speaker 111, microphone 113, touch screen 112, display controller 156, contact/motion module 130, graphics module 132, and text input module 134, telephone module 138 are optionally, used to enter a sequence of characters corresponding to a telephone number, access one or more telephone numbers in contacts module 137, modify a telephone number that has been entered, dial a respective telephone number, conduct a conversation, and disconnect or hang up when the conversation is completed. As noted above, the wireless communication optionally uses any of a plurality of communications standards, protocols, and technologies.

In conjunction with RF circuitry 108, audio circuitry 110, speaker 111, microphone 113, touch screen 112, display controller 156, optical sensor 164, optical sensor controller 158, contact/motion module 130, graphics module 132, text input module 134, contacts module 137, and telephone module 138, video conference module 139 includes executable instructions to initiate, conduct, and terminate a video conference between a user and one or more other participants in accordance with user instructions.

In conjunction with RF circuitry 108, touch screen 112, display controller 156, contact/motion module 130, graphics module 132, and text input module 134, e-mail client module 140 includes executable instructions to create, send, receive, and manage e-mail in response to user instructions. In conjunction with image management module 144, e-mail client module 140 makes it very easy to create and send e-mails with still or video images taken with camera module 143.

In conjunction with RF circuitry 108, touch screen 112, display controller 156, contact/motion module 130, graphics module 132, and text input module 134, the instant messaging module 141 includes executable instructions to enter a sequence of characters corresponding to an instant message, to modify previously entered characters, to transmit a respective instant message (for example, using a Short Message Service (SMS) or Multimedia Message Service (MMS) protocol for telephony-based instant messages or using XMPP, SIMPLE, or IMPS for Internet-based instant messages), to receive instant messages, and to view received instant messages. In some embodiments, transmitted and/or received instant messages optionally include graphics, photos, audio files, video files and/or other attachments as are supported in an MMS and/or an Enhanced Messaging Service (EMS). As used herein, “instant messaging” refers to both telephony-based messages (e.g., messages sent using SMS or MMS) and Internet-based messages (e.g., messages sent using XMPP, SIMPLE, or IMPS).

In conjunction with RF circuitry 108, touch screen 112, display controller 156, contact/motion module 130, graphics module 132, text input module 134, GPS module 135, map module 154, and music player module, workout support module 142 includes executable instructions to create workouts (e.g., with time, distance, and/or calorie burning goals); communicate with workout sensors (sports devices); receive workout sensor data; calibrate sensors used to monitor a workout; select and play music for a workout; and display, store, and transmit workout data.

In conjunction with touch screen 112, display controller 156, optical sensor(s) 164, optical sensor controller 158, contact/motion module 130, graphics module 132, and image management module 144, camera module 143 includes executable instructions to capture still images or video (including a video stream) and store them into memory 102, modify characteristics of a still image or video, or delete a still image or video from memory 102.

In conjunction with touch screen 112, display controller 156, contact/motion module 130, graphics module 132, text input module 134, and camera module 143, image management module 144 includes executable instructions to arrange, modify (e.g., edit), or otherwise manipulate, label, delete, present (e.g., in a digital slide show or album), and store still and/or video images.

In conjunction with RF circuitry 108, touch screen 112, display controller 156, contact/motion module 130, graphics module 132, and text input module 134, browser module 147 includes executable instructions to browse the Internet in accordance with user instructions, including searching, linking to, receiving, and displaying web pages or portions thereof, as well as attachments and other files linked to web pages.

In conjunction with RF circuitry 108, touch screen 112, display controller 156, contact/motion module 130, graphics module 132, text input module 134, e-mail client module 140, and browser module 147, calendar module 148 includes executable instructions to create, display, modify, and store calendars and data associated with calendars (e.g., calendar entries, to-do lists, etc.) in accordance with user instructions.

In conjunction with RF circuitry 108, touch screen 112, display controller 156, contact/motion module 130, graphics module 132, text input module 134, and browser module 147, widget modules 149 are mini-applications that are, optionally, downloaded and used by a user (e.g., weather widget 149-1, stocks widget 149-2, calculator widget 149-3, alarm clock widget 149-4, and dictionary widget 149-5) or created by the user (e.g., user-created widget 149-6). In some embodiments, a widget includes an HTML (Hypertext Markup Language) file, a CSS (Cascading Style Sheets) file, and a JavaScript file. In some embodiments, a widget includes an XML (Extensible Markup Language) file and a JavaScript file (e.g., Yahoo! Widgets).

In conjunction with RF circuitry 108, touch screen 112, display controller 156, contact/motion module 130, graphics module 132, text input module 134, and browser module 147, the widget creator module 150 are, optionally, used by a user to create widgets (e.g., turning a user-specified portion of a web page into a widget).

In conjunction with touch screen 112, display controller 156, contact/motion module 130, graphics module 132, and text input module 134, search module 151 includes executable instructions to search for text, music, sound, image, video, and/or other files in memory 102 that match one or more search criteria (e.g., one or more user-specified search terms) in accordance with user instructions.

In conjunction with touch screen 112, display controller 156, contact/motion module 130, graphics module 132, audio circuitry 110, speaker 111, RF circuitry 108, and browser module 147, video and music player module 152 includes executable instructions that allow the user to download and play back recorded music and other sound files stored in one or more file formats, such as MP3 or AAC files, and executable instructions to display, present, or otherwise play back videos (e.g., on touch screen 112 or on an external, connected display via external port 124). In some embodiments, device 100 optionally includes the functionality of an MP3 player, such as an iPod (trademark of Apple Inc.).

In conjunction with touch screen 112, display controller 156, contact/motion module 130, graphics module 132, and text input module 134, notes module 153 includes executable instructions to create and manage notes, to-do lists, and the like in accordance with user instructions.

In conjunction with RF circuitry 108, touch screen 112, display controller 156, contact/motion module 130, graphics module 132, text input module 134, GPS module 135, and browser module 147, map module 154 are, optionally, used to receive, display, modify, and store maps and data associated with maps (e.g., driving directions, data on stores and other points of interest at or near a particular location, and other location-based data) in accordance with user instructions.

In conjunction with touch screen 112, display controller 156, contact/motion module 130, graphics module 132, audio circuitry 110, speaker 111, RF circuitry 108, text input module 134, e-mail client module 140, and browser module 147, online video module 155 includes instructions that allow the user to access, browse, receive (e.g., by streaming and/or download), play back (e.g., on the touch screen or on an external, connected display via external port 124), send an e-mail with a link to a particular online video, and otherwise manage online videos in one or more file formats, such as H.264. In some embodiments, instant messaging module 141, rather than e-mail client module 140, is used to send a link to a particular online video. Additional description of the online video application can be found in U.S. Provisional Patent Application No. 60/936,562, “Portable Multifunction Device, Method, and Graphical User Interface for Playing Online Videos,” filed Jun. 20, 2007, and U.S. patent application Ser. No. 11/968,067, “Portable Multifunction Device, Method, and Graphical User Interface for Playing Online Videos,” filed Dec. 31, 2007, the contents of which are hereby incorporated by reference in their entirety.

Each of the above-identified modules and applications corresponds to a set of executable instructions for performing one or more functions described above and the methods described in this application (e.g., the computer-implemented methods and other information processing methods described herein). These modules (e.g., sets of instructions) need not be implemented as separate software programs (such as computer programs (e.g., including instructions)), procedures, or modules, and thus various subsets of these modules are, optionally, combined or otherwise rearranged in various embodiments. For example, video player module is, optionally, combined with music player module into a single module (e.g., video and music player module 152, FIG. 1A). In some embodiments, memory 102 optionally stores a subset of the modules and data structures identified above. Furthermore, memory 102 optionally stores additional modules and data structures not described above.

In some embodiments, device 100 is a device where operation of a predefined set of functions on the device is performed exclusively through a touch screen and/or a touchpad. By using a touch screen and/or a touchpad as the primary input control device for operation of device 100, the number of physical input control devices (such as push buttons, dials, and the like) on device 100 is, optionally, reduced.

The predefined set of functions that are performed exclusively through a touch screen and/or a touchpad optionally include navigation between user interfaces. In some embodiments, the touchpad, when touched by the user, navigates device 100 to a main, home, or root menu from any user interface that is displayed on device 100. In such embodiments, a “menu button” is implemented using a touchpad. In some other embodiments, the menu button is a physical push button or other physical input control device instead of a touchpad.

FIG. 1B is a block diagram illustrating exemplary components for event handling in accordance with some embodiments. In some embodiments, memory 102 (FIG. 1A) or 370 (FIG. 3A) includes event sorter 170 (e.g., in operating system 126) and a respective application 136-1 (e.g., any of the aforementioned applications 137-151, 155, 380-390).

Event sorter 170 receives event information and determines the application 136-1 and application view 191 of application 136-1 to which to deliver the event information. Event sorter 170 includes event monitor 171 and event dispatcher module 174. In some embodiments, application 136-1 includes application internal state 192, which indicates the current application view(s) displayed on touch-sensitive display 112 when the application is active or executing. In some embodiments, device/global internal state 157 is used by event sorter 170 to determine which application(s) is (are) currently active, and application internal state 192 is used by event sorter 170 to determine application views 191 to which to deliver event information.

In some embodiments, application internal state 192 includes additional information, such as one or more of: resume information to be used when application 136-1 resumes execution, user interface state information that indicates information being displayed or that is ready for display by application 136-1, a state queue for enabling the user to go back to a prior state or view of application 136-1, and a redo/undo queue of previous actions taken by the user.

Event monitor 171 receives event information from peripherals interface 118. Event information includes information about a sub-event (e.g., a user touch on touch-sensitive display 112, as part of a multi-touch gesture). Peripherals interface 118 transmits information it receives from I/O subsystem 106 or a sensor, such as proximity sensor 166, accelerometer(s) 168, and/or microphone 113 (through audio circuitry 110). Information that peripherals interface 118 receives from I/O subsystem 106 includes information from touch-sensitive display 112 or a touch-sensitive surface.

In some embodiments, event monitor 171 sends requests to the peripherals interface 118 at predetermined intervals. In response, peripherals interface 118 transmits event information. In other embodiments, peripherals interface 118 transmits event information only when there is a significant event (e.g., receiving an input above a predetermined noise threshold and/or for more than a predetermined duration).

In some embodiments, event sorter 170 also includes a hit view determination module 172 and/or an active event recognizer determination module 173.

Hit view determination module 172 provides software procedures for determining where a sub-event has taken place within one or more views when touch-sensitive display 112 displays more than one view. Views are made up of controls and other elements that a user can see on the display.

Another aspect of the user interface associated with an application is a set of views, sometimes herein called application views or user interface windows, in which information is displayed and touch-based gestures occur. The application views (of a respective application) in which a touch is detected optionally correspond to programmatic levels within a programmatic or view hierarchy of the application. For example, the lowest level view in which a touch is detected is, optionally, called the hit view, and the set of events that are recognized as proper inputs are, optionally, determined based, at least in part, on the hit view of the initial touch that begins a touch-based gesture.

Hit view determination module 172 receives information related to sub-events of a touch-based gesture. When an application has multiple views organized in a hierarchy, hit view determination module 172 identifies a hit view as the lowest view in the hierarchy which should handle the sub-event. In most circumstances, the hit view is the lowest level view in which an initiating sub-event occurs (e.g., the first sub-event in the sequence of sub-events that form an event or potential event). Once the hit view is identified by the hit view determination module 172, the hit view typically receives all sub-events related to the same touch or input source for which it was identified as the hit view.

Active event recognizer determination module 173 determines which view or views within a view hierarchy should receive a particular sequence of sub-events. In some embodiments, active event recognizer determination module 173 determines that only the hit view should receive a particular sequence of sub-events. In other embodiments, active event recognizer determination module 173 determines that all views that include the physical location of a sub-event are actively involved views, and therefore determines that all actively involved views should receive a particular sequence of sub-events. In other embodiments, even if touch sub-events were entirely confined to the area associated with one particular view, views higher in the hierarchy would still remain as actively involved views.

Event dispatcher module 174 dispatches the event information to an event recognizer (e.g., event recognizer 180). In embodiments including active event recognizer determination module 173, event dispatcher module 174 delivers the event information to an event recognizer determined by active event recognizer determination module 173. In some embodiments, event dispatcher module 174 stores in an event queue the event information, which is retrieved by a respective event receiver 182.

In some embodiments, operating system 126 includes event sorter 170. Alternatively, application 136-1 includes event sorter 170. In yet other embodiments, event sorter 170 is a stand-alone module, or a part of another module stored in memory 102, such as contact/motion module 130.

In some embodiments, application 136-1 includes a plurality of event handlers 190 and one or more application views 191, each of which includes instructions for handling touch events that occur within a respective view of the application's user interface. Each application view 191 of the application 136-1 includes one or more event recognizers 180. Typically, a respective application view 191 includes a plurality of event recognizers 180. In other embodiments, one or more of event recognizers 180 are part of a separate module, such as a user interface kit or a higher level object from which application 136-1 inherits methods and other properties. In some embodiments, a respective event handler 190 includes one or more of: data updater 176, object updater 177, GUI updater 178, and/or event data 179 received from event sorter 170. Event handler 190 optionally utilizes or calls data updater 176, object updater 177, or GUI updater 178 to update the application internal state 192. Alternatively, one or more of the application views 191 include one or more respective event handlers 190. Also, in some embodiments, one or more of data updater 176, object updater 177, and GUI updater 178 are included in a respective application view 191.

A respective event recognizer 180 receives event information (e.g., event data 179) from event sorter 170 and identifies an event from the event information. Event recognizer 180 includes event receiver 182 and event comparator 184. In some embodiments, event recognizer 180 also includes at least a subset of: metadata 183, and event delivery instructions 188 (which optionally include sub-event delivery instructions).

Event receiver 182 receives event information from event sorter 170. The event information includes information about a sub-event, for example, a touch or a touch movement. Depending on the sub-event, the event information also includes additional information, such as location of the sub-event. When the sub-event concerns motion of a touch, the event information optionally also includes speed and direction of the sub-event. In some embodiments, events include rotation of the device from one orientation to another (e.g., from a portrait orientation to a landscape orientation, or vice versa), and the event information includes corresponding information about the current orientation (also called device attitude) of the device.

Event comparator 184 compares the event information to predefined event or sub-event definitions and, based on the comparison, determines an event or sub-event, or determines or updates the state of an event or sub-event. In some embodiments, event comparator 184 includes event definitions 186. Event definitions 186 contain definitions of events (e.g., predefined sequences of sub-events), for example, event 1 (187-1), event 2 (187-2), and others. In some embodiments, sub-events in an event (e.g., 187-1 and/or 187-2) include, for example, touch begin, touch end, touch movement, touch cancellation, and multiple touching. In one example, the definition for event 1 (187-1) is a double tap on a displayed object. The double tap, for example, comprises a first touch (touch begin) on the displayed object for a predetermined phase, a first liftoff (touch end) for a predetermined phase, a second touch (touch begin) on the displayed object for a predetermined phase, and a second liftoff (touch end) for a predetermined phase. In another example, the definition for event 2 (187-2) is a dragging on a displayed object. The dragging, for example, comprises a touch (or contact) on the displayed object for a predetermined phase, a movement of the touch across touch-sensitive display 112, and liftoff of the touch (touch end). In some embodiments, the event also includes information for one or more associated event handlers 190.

In some embodiments, event definitions 186 include a definition of an event for a respective user-interface object. In some embodiments, event comparator 184 performs a hit test to determine which user-interface object is associated with a sub-event. For example, in an application view in which three user-interface objects are displayed on touch-sensitive display 112, when a touch is detected on touch-sensitive display 112, event comparator 184 performs a hit test to determine which of the three user-interface objects is associated with the touch (sub-event). If each displayed object is associated with a respective event handler 190, the event comparator uses the result of the hit test to determine which event handler 190 should be activated. For example, event comparator 184 selects an event handler associated with the sub-event and the object triggering the hit test.

In some embodiments, the definition for a respective event (187) also includes delayed actions that delay delivery of the event information until after it has been determined whether the sequence of sub-events does or does not correspond to the event recognizer's event type.

When a respective event recognizer 180 determines that the series of sub-events do not match any of the events in event definitions 186, the respective event recognizer 180 enters an event impossible, event failed, or event ended state, after which it disregards subsequent sub-events of the touch-based gesture. In this situation, other event recognizers, if any, that remain active for the hit view continue to track and process sub-events of an ongoing touch-based gesture.

In some embodiments, a respective event recognizer 180 includes metadata 183 with configurable properties, flags, and/or lists that indicate how the event delivery system should perform sub-event delivery to actively involved event recognizers. In some embodiments, metadata 183 includes configurable properties, flags, and/or lists that indicate how event recognizers interact, or are enabled to interact, with one another. In some embodiments, metadata 183 includes configurable properties, flags, and/or lists that indicate whether sub-events are delivered to varying levels in the view or programmatic hierarchy.

In some embodiments, a respective event recognizer 180 activates event handler 190 associated with an event when one or more particular sub-events of an event are recognized. In some embodiments, a respective event recognizer 180 delivers event information associated with the event to event handler 190. Activating an event handler 190 is distinct from sending (and deferred sending) sub-events to a respective hit view. In some embodiments, event recognizer 180 throws a flag associated with the recognized event, and event handler 190 associated with the flag catches the flag and performs a predefined process.

In some embodiments, event delivery instructions 188 include sub-event delivery instructions that deliver event information about a sub-event without activating an event handler. Instead, the sub-event delivery instructions deliver event information to event handlers associated with the series of sub-events or to actively involved views. Event handlers associated with the series of sub-events or with actively involved views receive the event information and perform a predetermined process.

In some embodiments, data updater 176 creates and updates data used in application 136-1. For example, data updater 176 updates the telephone number used in contacts module 137, or stores a video file used in video player module. In some embodiments, object updater 177 creates and updates objects used in application 136-1. For example, object updater 177 creates a new user-interface object or updates the position of a user-interface object. GUI updater 178 updates the GUI. For example, GUI updater 178 prepares display information and sends it to graphics module 132 for display on a touch-sensitive display.

In some embodiments, event handler(s) 190 includes or has access to data updater 176, object updater 177, and GUI updater 178. In some embodiments, data updater 176, object updater 177, and GUI updater 178 are included in a single module of a respective application 136-1 or application view 191. In other embodiments, they are included in two or more software modules.

It shall be understood that the foregoing discussion regarding event handling of user touches on touch-sensitive displays also applies to other forms of user inputs to operate multifunction devices 100 with input devices, not all of which are initiated on touch screens. For example, mouse movement and mouse button presses, optionally coordinated with single or multiple keyboard presses or holds; contact movements such as taps, drags, scrolls, etc. on touchpads; pen stylus inputs; movement of the device; oral instructions; detected eye movements; biometric inputs; and/or any combination thereof are optionally utilized as inputs corresponding to sub-events which define an event to be recognized.

FIG. 2 illustrates a portable multifunction device 100 having a touch screen 112 in accordance with some embodiments. The touch screen optionally displays one or more graphics within user interface (UI) 200. In this embodiment, as well as others described below, a user is enabled to select one or more of the graphics by making a gesture on the graphics, for example, with one or more fingers 202 (not drawn to scale in the figure) or one or more styluses 203 (not drawn to scale in the figure). In some embodiments, selection of one or more graphics occurs when the user breaks contact with the one or more graphics. In some embodiments, the gesture optionally includes one or more taps, one or more swipes (from left to right, right to left, upward and/or downward), and/or a rolling of a finger (from right to left, left to right, upward and/or downward) that has made contact with device 100. In some implementations or circumstances, inadvertent contact with a graphic does not select the graphic. For example, a swipe gesture that sweeps over an application icon optionally does not select the corresponding application when the gesture corresponding to selection is a tap.

Device 100 optionally also include one or more physical buttons, such as “home” or menu button 204. As described previously, menu button 204 is, optionally, used to navigate to any application 136 in a set of applications that are, optionally, executed on device 100. Alternatively, in some embodiments, the menu button is implemented as a soft key in a GUI displayed on touch screen 112.

In some embodiments, device 100 includes touch screen 112, menu button 204, push button 206 for powering the device on/off and locking the device, volume adjustment button(s) 208, subscriber identity module (SIM) card slot 210, headset jack 212, and docking/charging external port 124. Push button 206 is, optionally, used to turn the power on/off on the device by depressing the button and holding the button in the depressed state for a predefined time interval; to lock the device by depressing the button and releasing the button before the predefined time interval has elapsed; and/or to unlock the device or initiate an unlock process. In an alternative embodiment, device 100 also accepts verbal input for activation or deactivation of some functions through microphone 113. Device 100 also, optionally, includes one or more contact intensity sensors 165 for detecting intensity of contacts on touch screen 112 and/or one or more tactile output generators 167 for generating tactile outputs for a user of device 100.

FIG. 3A is a block diagram of an exemplary multifunction device with a display and a touch-sensitive surface in accordance with some embodiments. Device 300 need not be portable. In some embodiments, device 300 is a laptop computer, a desktop computer, a tablet computer, a multimedia player device, a navigation device, an educational device (such as a child's learning toy), a gaming system, or a control device (e.g., a home or industrial controller). Device 300 typically includes one or more processing units (CPUs) 310, one or more network or other communications interfaces 360, memory 370, and one or more communication buses 320 for interconnecting these components. Communication buses 320 optionally include circuitry (sometimes called a chipset) that interconnects and controls communications between system components. Device 300 includes input/output (I/O) interface 330 comprising display 340, which is typically a touch screen display. I/O interface 330 also optionally includes a keyboard and/or mouse (or other pointing device) 350 and touchpad 355, tactile output generator 357 for generating tactile outputs on device 300 (e.g., similar to tactile output generator(s) 167 described above with reference to FIG. 1A), sensors 359 (e.g., optical, acceleration, proximity, touch-sensitive, and/or contact intensity sensors similar to contact intensity sensor(s) 165 described above with reference to FIG. 1A). Memory 370 includes high-speed random access memory, such as DRAM, SRAM, DDR RAM, or other random access solid state memory devices; and optionally includes non-volatile memory, such as one or more magnetic disk storage devices, optical disk storage devices, flash memory devices, or other non-volatile solid state storage devices. Memory 370 optionally includes one or more storage devices remotely located from CPU(s) 310. In some embodiments, memory 370 stores programs, modules, and data structures analogous to the programs, modules, and data structures stored in memory 102 of portable multifunction device 100 (FIG. 1A), or a subset thereof. Furthermore, memory 370 optionally stores additional programs, modules, and data structures not present in memory 102 of portable multifunction device 100. For example, memory 370 of device 300 optionally stores drawing module 380, presentation module 382, word processing module 384, website creation module 386, disk authoring module 388, and/or spreadsheet module 390, while memory 102 of portable multifunction device 100 (FIG. 1A) optionally does not store these modules.

Each of the above-identified elements in FIG. 3A is, optionally, stored in one or more of the previously mentioned memory devices. Each of the above-identified modules corresponds to a set of instructions for performing a function described above. The above-identified modules or computer programs (e.g., sets of instructions or including instructions) need not be implemented as separate software programs (such as computer programs (e.g., including instructions)), procedures, or modules, and thus various subsets of these modules are, optionally, combined or otherwise rearranged in various embodiments. In some embodiments, memory 370 optionally stores a subset of the modules and data structures identified above. Furthermore, memory 370 optionally stores additional modules and data structures not described above.

Implementations within the scope of the present disclosure can be partially or entirely realized using a tangible computer-readable storage medium (or multiple tangible computer-readable storage media of one or more types) encoding one or more computer-readable instructions. It should be recognized that computer-readable instructions can be organized in any format, including applications, widgets, processes, software, and/or components.

Implementations within the scope of the present disclosure include a computer-readable storage medium that encodes instructions organized as an application (e.g., application 3160) that, when executed by one or more processing units, control an electronic device (e.g., device 3150) to perform the method of FIG. 3B, the method of FIG. 3C, and/or one or more other processes and/or methods described herein.

It should be recognized that application 3160 (shown in FIG. 3D) can be any suitable type of application, including, for example, one or more of: a browser application, an application that functions as an execution environment for plug-ins, widgets or other applications, a fitness application, a health application, a digital payments application, a media application, a social network application, a messaging application, and/or a maps application. In some embodiments, application 3160 is an application that is pre-installed on device 3150 at purchase (e.g., a first-party application). In some embodiments, application 3160 is an application that is provided to device 3150 via an operating system update file (e.g., a first-party application or a second-party application). In some embodiments, application 3160 is an application that is provided via an application store. In some embodiments, the application store can be an application store that is pre-installed on device 3150 at purchase (e.g., a first-party application store). In some embodiments, the application store is a third-party application store (e.g., an application store that is provided by another application store, downloaded via a network, and/or read from a storage device).

Referring to FIG. 3B and FIG. 3F, application 3160 obtains information (e.g., 3010). In some embodiments, at 3010, information is obtained from at least one hardware component of device 3150. In some embodiments, at 3010, information is obtained from at least one software module of device 3150. In some embodiments, at 3010, information is obtained from at least one hardware component external to device 3150 (e.g., a peripheral device, an accessory device, and/or a server). In some embodiments, the information obtained at 3010 includes positional information, time information, notification information, user information, environment information, electronic device state information, weather information, media information, historical information, event information, hardware information, and/or motion information. In some embodiments, in response to and/or after obtaining the information at 3010, application 3160 provides the information to a system (e.g., 3020).

In some embodiments, the system (e.g., 3110 shown in FIG. 3E) is an operating system hosted on device 3150. In some embodiments, the system (e.g., 3110 shown in FIG. 3E) is an external device (e.g., a server, a peripheral device, an accessory, and/or a personal computing device) that includes an operating system.

Referring to FIG. 3C and FIG. 3G, application 3160 obtains information (e.g., 3030). In some embodiments, the information obtained at 3030 includes positional information, time information, notification information, user information, environment information electronic device state information, weather information, media information, historical information, event information, hardware information, and/or motion information. In response to and/or after obtaining the information at 3030, application 3160 performs an operation with the information (e.g., 3040). In some embodiments, the operation performed at 3040 includes: providing a notification based on the information, sending a message based on the information, displaying the information, controlling a user interface of a fitness application based on the information, controlling a user interface of a health application based on the information, controlling a focus mode based on the information, setting a reminder based on the information, adding a calendar entry based on the information, and/or calling an API of system 3110 based on the information.

In some embodiments, one or more steps of the method of FIG. 3B and/or the method of FIG. 3C is performed in response to a trigger. In some embodiments, the trigger includes detection of an event, a notification received from system 3110, a user input, and/or a response to a call to an API provided by system 3110.

In some embodiments, the instructions of application 3160, when executed, control device 3150 to perform the method of FIG. 3B and/or the method of FIG. 3C by calling an application programming interface (API) (e.g., API 3190) provided by system 3110. In some embodiments, application 3160 performs at least a portion of the method of FIG. 3B and/or the method of FIG. 3C without calling API 3190.

In some embodiments, one or more steps of the method of FIG. 3B and/or the method of FIG. 3C includes calling an API (e.g., API 3190) using one or more parameters defined by the API. In some embodiments, the one or more parameters include a constant, a key, a data structure, an object, an object class, a variable, a data type, a pointer, an array, a list or a pointer to a function or method, and/or another way to reference a data or other item to be passed via the API.

Referring to FIG. 3D, device 3150 is illustrated. In some embodiments, device 3150 is a personal computing device, a smart phone, a smart watch, a fitness tracker, a head mounted display (HMD) device, a media device, a communal device, a speaker, a television, and/or a tablet. As illustrated in FIG. 3D, device 3150 includes application 3160 and an operating system (e.g., system 3110 shown in FIG. 3E). Application 3160 includes application implementation module 3170 and API-calling module 3180. System 3110 includes API 3190 and implementation module 3100. It should be recognized that device 3150, application 3160, and/or system 3110 can include more, fewer, and/or different components than illustrated in FIGS. 3D and 3E.

In some embodiments, application implementation module 3170 includes a set of one or more instructions corresponding to one or more operations performed by application 3160. For example, when application 3160 is a messaging application, application implementation module 3170 can include operations to receive and send messages. In some embodiments, application implementation module 3170 communicates with API-calling module 3180 to communicate with system 3110 via API 3190 (shown in FIG. 3E).

In some embodiments, API 3190 is a software module (e.g., a collection of computer-readable instructions) that provides an interface that allows a different module (e.g., API-calling module 3180) to access and/or use one or more functions, methods, procedures, data structures, classes, and/or other services provided by implementation module 3100 of system 3110. For example, API-calling module 3180 can access a feature of implementation module 3100 through one or more API calls or invocations (e.g., embodied by a function or a method call) exposed by API 3190 (e.g., a software and/or hardware module that can receive API calls, respond to API calls, and/or send API calls) and can pass data and/or control information using one or more parameters via the API calls or invocations. In some embodiments, API 3190 allows application 3160 to use a service provided by a Software Development Kit (SDK) library. In some embodiments, application 3160 incorporates a call to a function or method provided by the SDK library and provided by API 3190 or uses data types or objects defined in the SDK library and provided by API 3190. In some embodiments, API-calling module 3180 makes an API call via API 3190 to access and use a feature of implementation module 3100 that is specified by API 3190. In such embodiments, implementation module 3100 can return a value via API 3190 to API-calling module 3180 in response to the API call. The value can report to application 3160 the capabilities or state of a hardware component of device 3150, including those related to aspects such as input capabilities and state, output capabilities and state, processing capability, power state, storage capacity and state, and/or communications capability. In some embodiments, API 3190 is implemented in part by firmware, microcode, or other low level logic that executes in part on the hardware component.

In some embodiments, API 3190 allows a developer of API-calling module 3180 (which can be a third-party developer) to leverage a feature provided by implementation module 3100. In such embodiments, there can be one or more API-calling modules (e.g., including API-calling module 3180) that communicate with implementation module 3100. In some embodiments, API 3190 allows multiple API-calling modules written in different programming languages to communicate with implementation module 3100 (e.g., API 3190 can include features for translating calls and returns between implementation module 3100 and API-calling module 3180) while API 3190 is implemented in terms of a specific programming language. In some embodiments, API-calling module 3180 calls APIs from different providers such as a set of APIs from an OS provider, another set of APIs from a plug-in provider, and/or another set of APIs from another provider (e.g., the provider of a software library) or creator of the another set of APIs.

Examples of API 3190 can include one or more of: a pairing API (e.g., for establishing secure connection, e.g., with an accessory), a device detection API (e.g., for locating nearby devices, e.g., media devices and/or smartphone), a payment API, a UIKit API (e.g., for generating user interfaces), a location detection API, a locator API, a maps API, a health sensor API, a sensor API, a messaging API, a push notification API, a streaming API, a collaboration API, a video conferencing API, an application store API, an advertising services API, a web browser API (e.g., WebKit API), a vehicle API, a networking API, a WiFi API, a Bluetooth API, an NFC API, a UWB API, a fitness API, a smart home API, contact transfer API, photos API, camera API, and/or image processing API. In some embodiments, the sensor API is an API for accessing data associated with a sensor of device 3150. For example, the sensor API can provide access to raw sensor data. For another example, the sensor API can provide data derived (and/or generated) from the raw sensor data. In some embodiments, the sensor data includes temperature data, image data, video data, audio data, heart rate data, IMU (inertial measurement unit) data, lidar data, location data, GPS data, and/or camera data. In some embodiments, the sensor includes one or more of an accelerometer, temperature sensor, infrared sensor, optical sensor, heartrate sensor, barometer, gyroscope, proximity sensor, temperature sensor, and/or biometric sensor.

In some embodiments, implementation module 3100 is a system (e.g., operating system and/or server system) software module (e.g., a collection of computer-readable instructions) that is constructed to perform an operation in response to receiving an API call via API 3190. In some embodiments, implementation module 3100 is constructed to provide an API response (via API 3190) as a result of processing an API call. By way of example, implementation module 3100 and API-calling module 3180 can each be any one of an operating system, a library, a device driver, an API, an application program, or other module. It should be understood that implementation module 3100 and API-calling module 3180 can be the same or different type of module from each other. In some embodiments, implementation module 3100 is embodied at least in part in firmware, microcode, or hardware logic.

In some embodiments, implementation module 3100 returns a value through API 3190 in response to an API call from API-calling module 3180. While API 3190 defines the syntax and result of an API call (e.g., how to invoke the API call and what the API call does), API 3190 might not reveal how implementation module 3100 accomplishes the function specified by the API call. Various API calls are transferred via the one or more application programming interfaces between API-calling module 3180 and implementation module 3100. Transferring the API calls can include issuing, initiating, invoking, calling, receiving, returning, and/or responding to the function calls or messages. In other words, transferring can describe actions by either of API-calling module 3180 or implementation module 3100. In some embodiments, a function call or other invocation of API 3190 sends and/or receives one or more parameters through a parameter list or other structure.

In some embodiments, implementation module 3100 provides more than one API, each providing a different view of or with different aspects of functionality implemented by implementation module 3100. For example, one API of implementation module 3100 can provide a first set of functions and can be exposed to third-party developers, and another API of implementation module 3100 can be hidden (e.g., not exposed) and provide a subset of the first set of functions and also provide another set of functions, such as testing or debugging functions which are not in the first set of functions. In some embodiments, implementation module 3100 calls one or more other components via an underlying API and thus is both an API-calling module and an implementation module. It should be recognized that implementation module 3100 can include additional functions, methods, classes, data structures, and/or other features that are not specified through API 3190 and are not available to API-calling module 3180. It should also be recognized that API-calling module 3180 can be on the same system as implementation module 3100 or can be located remotely and access implementation module 3100 using API 3190 over a network. In some embodiments, implementation module 3100, API 3190, and/or API-calling module 3180 is stored in a machine-readable medium, which includes any mechanism for storing information in a form readable by a machine (e.g., a computer or other data processing system). For example, a machine-readable medium can include magnetic disks, optical disks, random access memory; read only memory, and/or flash memory devices.

An application programming interface (API) is an interface between a first software process and a second software process that specifies a format for communication between the first software process and the second software process. Limited APIs (e.g., private APIs or partner APIs) are APIs that are accessible to a limited set of software processes (e.g., only software processes within an operating system or only software processes that are approved to access the limited APIs). Public APIs that are accessible to a wider set of software processes. Some APIs enable software processes to communicate about or set a state of one or more input devices (e.g., one or more touch sensors, proximity sensors, visual sensors, motion/orientation sensors, pressure sensors, intensity sensors, sound sensors, wireless proximity sensors, biometric sensors, buttons, switches, rotatable elements, and/or external controllers). Some APIs enable software processes to communicate about and/or set a state of one or more output generation components (e.g., one or more audio output generation components, one or more display generation components, and/or one or more tactile output generation components). Some APIs enable particular capabilities (e.g., scrolling, handwriting, text entry, image editing, and/or image creation) to be accessed, performed, and/or used by a software process (e.g., generating outputs for use by a software process based on input from the software process). Some APIs enable content from a software process to be inserted into a template and displayed in a user interface that has a layout and/or behaviors that are specified by the template.

Many software platforms include a set of frameworks that provides the core objects and core behaviors that a software developer needs to build software applications that can be used on the software platform. Software developers use these objects to display content onscreen, to interact with that content, and to manage interactions with the software platform. Software applications rely on the set of frameworks for their basic behavior, and the set of frameworks provides many ways for the software developer to customize the behavior of the application to match the specific needs of the software application. Many of these core objects and core behaviors are accessed via an API. An API will typically specify a format for communication between software processes, including specifying and grouping available variables, functions, and protocols. An API call (sometimes referred to as an API request) will typically be sent from a sending software process to a receiving software process as a way to accomplish one or more of the following: the sending software process requesting information from the receiving software process (e.g., for the sending software process to take action on), the sending software process providing information to the receiving software process (e.g., for the receiving software process to take action on), the sending software process requesting action by the receiving software process, or the sending software process providing information to the receiving software process about action taken by the sending software process. Interaction with a device (e.g., using a user interface) will in some circumstances include the transfer and/or receipt of one or more API calls (e.g., multiple API calls) between multiple different software processes (e.g., different portions of an operating system, an application and an operating system, or different applications) via one or more APIs (e.g., via multiple different APIs). For example, when an input is detected the direct sensor data is frequently processed into one or more input events that are provided (e.g., via an API) to a receiving software process that makes some determination based on the input events, and then sends (e.g., via an API) information to a software process to perform an operation (e.g., change a device state and/or user interface) based on the determination. While a determination and an operation performed in response could be made by the same software process, alternatively the determination could be made in a first software process and relayed (e.g., via an API) to a second software process, that is different from the first software process, that causes the operation to be performed by the second software process. Alternatively, the second software process could relay instructions (e.g., via an API) to a third software process that is different from the first software process and/or the second software process to perform the operation. It should be understood that some or all user interactions with a computer system could involve one or more API calls within a step of interacting with the computer system (e.g., between different software components of the computer system or between a software component of the computer system and a software component of one or more remote computer systems). It should be understood that some or all user interactions with a computer system could involve one or more API calls between steps of interacting with the computer system (e.g., between different software components of the computer system or between a software component of the computer system and a software component of one or more remote computer systems).

In some embodiments, the application can be any suitable type of application, including, for example, one or more of: a browser application, an application that functions as an execution environment for plug-ins, widgets or other applications, a fitness application, a health application, a digital payments application, a media application, a social network application, a messaging application, and/or a maps application.

In some embodiments, the application is an application that is pre-installed on the first computer system at purchase (e.g., a first-party application). In some embodiments, the application is an application that is provided to the first computer system via an operating system update file (e.g., a first-party application). In some embodiments, the application is an application that is provided via an application store. In some embodiments, the application store is pre-installed on the first computer system at purchase (e.g., a first-party application store) and allows download of one or more applications. In some embodiments, the application store is a third-party application store (e.g., an application store that is provided by another device, downloaded via a network, and/or read from a storage device). In some embodiments, the application is a third-party application (e.g., an app that is provided by an application store, downloaded via a network, and/or read from a storage device). In some embodiments, the application controls the first computer system to perform methods 700, 800, and/or 1000 (FIGS. 7, 8, and/or 10) by calling an application programming interface (API) provided by the system process using one or more parameters.

In some embodiments, exemplary APIs provided by the system process include one or more of: a pairing API (e.g., for establishing secure connection, e.g., with an accessory), a device detection API (e.g., for locating nearby devices, e.g., media devices and/or smartphone), a payment API, a UIKit API (e.g., for generating user interfaces), a location detection API, a locator API, a maps API, a health sensor API, a sensor API, a messaging API, a push notification API, a streaming API, a collaboration API, a video conferencing API, an application store API, an advertising services API, a web browser API (e.g., WebKit API), a vehicle API, a networking API, a WiFi API, a Bluetooth API, an NFC API, a UWB API, a fitness API, a smart home API, contact transfer API, a photos API, a camera API, and/or an image processing API.

In some embodiments, at least one API is a software module (e.g., a collection of computer-readable instructions) that provides an interface that allows a different module (e.g., API-calling module) to access and use one or more functions, methods, procedures, data structures, classes, and/or other services provided by an implementation module of the system process. The API can define one or more parameters that are passed between the API-calling module and the implementation module. In some embodiments, API 3190 defines a first API call that can be provided by API-calling module 3180. The implementation module is a system software module (e.g., a collection of computer-readable instructions) that is constructed to perform an operation in response to receiving an API call via the API. In some embodiments, the implementation module is constructed to provide an API response (via the API) as a result of processing an API call. In some embodiments, the implementation module is included in the device (e.g., 3150) that runs the application. In some embodiments, the implementation module is included in an electronic device that is separate from the device that runs the application.

Attention is now directed towards embodiments of user interfaces that are, optionally, implemented on, for example, portable multifunction device 100.

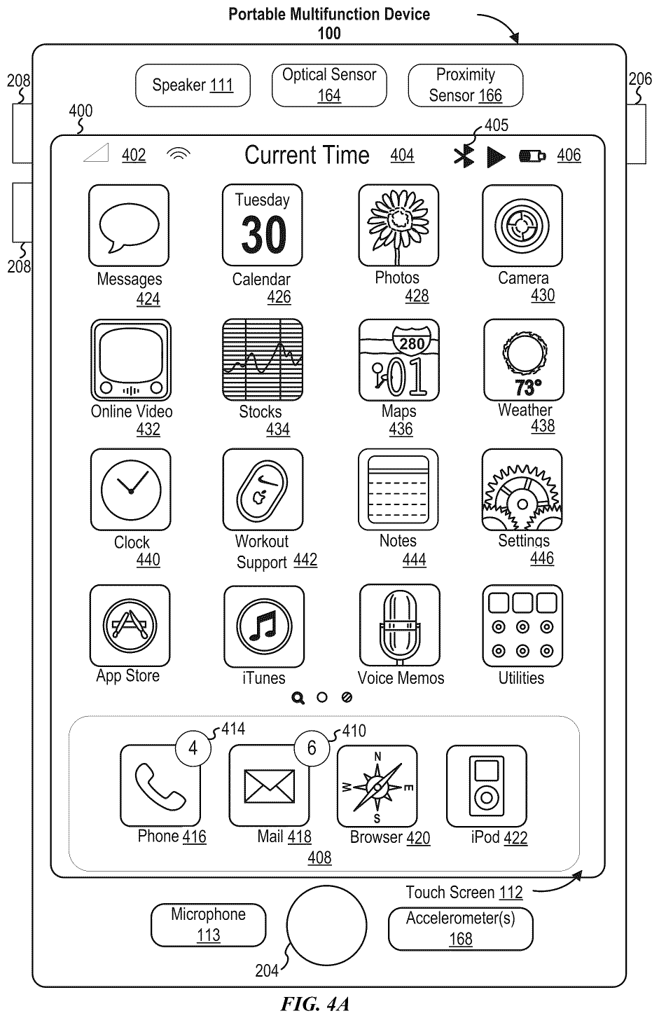

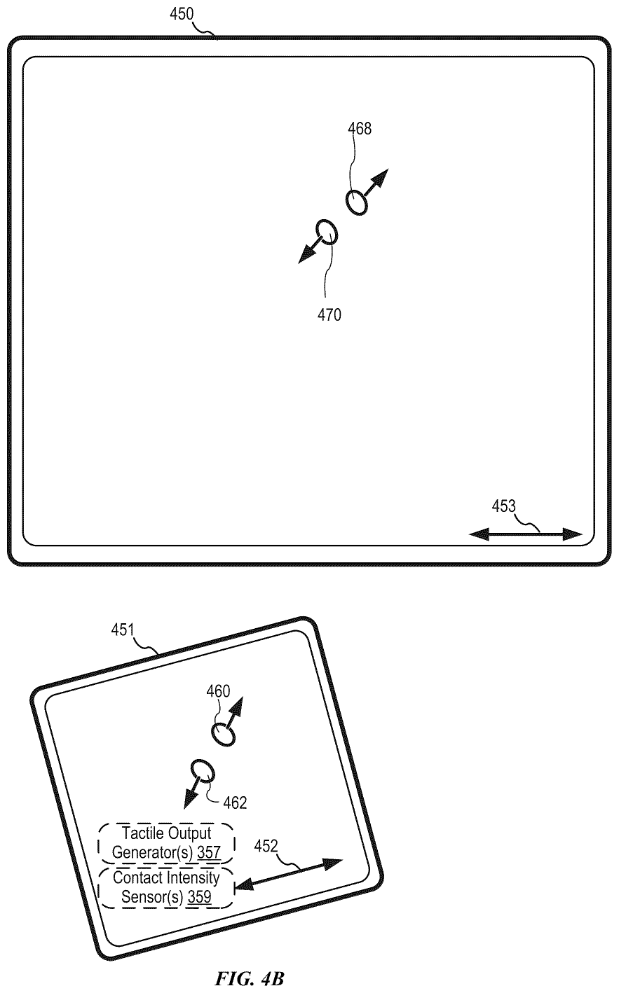

FIG. 4A illustrates an exemplary user interface for a menu of applications on portable multifunction device 100 in accordance with some embodiments. Similar user interfaces are, optionally, implemented on device 300. In some embodiments, user interface 400 includes the following elements, or a subset or superset thereof:
Signal strength indicator(s) 402 for wireless communication(s), such as cellular and Wi-Fi signals;
Time 404;
Bluetooth indicator 405;
Battery status indicator 406;
Tray 408 with icons for frequently used applications, such as:
   Icon 416 for telephone module 138, labeled “Phone,” which optionally includes an indicator 414 of the number of missed calls or voicemail messages;
   Icon 418 for e-mail client module 140, labeled “Mail,” which optionally includes an indicator 410 of the number of unread e-mails;
   Icon 420 for browser module 147, labeled “Browser;” and
   Icon 422 for video and music player module 152, also referred to as iPod (trademark of Apple Inc.) module 152, labeled “iPod;” and
Icons for other applications, such as:
   Icon 424 for IM module 141, labeled “Messages;”
   Icon 426 for calendar module 148, labeled “Calendar;”
   Icon 428 for image management module 144, labeled “Photos;”
   Icon 430 for camera module 143, labeled “Camera;”
   Icon 432 for online video module 155, labeled “Online Video;”
   Icon 434 for stocks widget 149-2, labeled “Stocks;”
   Icon 436 for map module 154, labeled “Maps;”
   Icon 438 for weather widget 149-1, labeled “Weather;”
   Icon 440 for alarm clock widget 149-4, labeled “Clock;”
   Icon 442 for workout support module 142, labeled “Workout Support;”
   Icon 444 for notes module 153, labeled “Notes;” and
   Icon 446 for a settings application or module, labeled “Settings,” which provides access to settings for device 100 and its various applications 136.

It should be noted that the icon labels illustrated in FIG. 4A are merely exemplary. For example, icon 422 for video and music player module 152 is labeled “Music” or “Music Player.” Other labels are, optionally, used for various application icons. In some embodiments, a label for a respective application icon includes a name of an application corresponding to the respective application icon. In some embodiments, a label for a particular application icon is distinct from a name of an application corresponding to the particular application icon.

FIG. 4B illustrates an exemplary user interface on a device (e.g., device 300, FIG. 3A) with a touch-sensitive surface 451 (e.g., a tablet or touchpad 355, FIG. 3A) that is separate from the display 450 (e.g., touch screen display 112). Device 300 also, optionally, includes one or more contact intensity sensors (e.g., one or more of sensors 359) for detecting intensity of contacts on touch-sensitive surface 451 and/or one or more tactile output generators 357 for generating tactile outputs for a user of device 300.

Although some of the examples that follow will be given with reference to inputs on touch screen display 112 (where the touch-sensitive surface and the display are combined), in some embodiments, the device detects inputs on a touch-sensitive surface that is separate from the display, as shown in FIG. 4B. In some embodiments, the touch-sensitive surface (e.g., 451 in FIG. 4B) has a primary axis (e.g., 452 in FIG. 4B) that corresponds to a primary axis (e.g., 453 in FIG. 4B) on the display (e.g., 450). In accordance with these embodiments, the device detects contacts (e.g., 460 and 462 in FIG. 4B) with the touch-sensitive surface 451 at locations that correspond to respective locations on the display (e.g., in FIG. 4B, 460 corresponds to 468 and 462 corresponds to 470). In this way, user inputs (e.g., contacts 460 and 462, and movements thereof) detected by the device on the touch-sensitive surface (e.g., 451 in FIG. 4B) are used by the device to manipulate the user interface on the display (e.g., 450 in FIG. 4B) of the multifunction device when the touch-sensitive surface is separate from the display. It should be understood that similar methods are, optionally, used for other user interfaces described herein.

Additionally, while the following examples are given primarily with reference to finger inputs (e.g., finger contacts, finger tap gestures, finger swipe gestures), it should be understood that, in some embodiments, one or more of the finger inputs are replaced with input from another input device (e.g., a mouse-based input or stylus input). For example, a swipe gesture is, optionally, replaced with a mouse click (e.g., instead of a contact) followed by movement of the cursor along the path of the swipe (e.g., instead of movement of the contact). As another example, a tap gesture is, optionally, replaced with a mouse click while the cursor is located over the location of the tap gesture (e.g., instead of detection of the contact followed by ceasing to detect the contact). Similarly, when multiple user inputs are simultaneously detected, it should be understood that multiple computer mice are, optionally, used simultaneously, or a mouse and finger contacts are, optionally, used simultaneously.

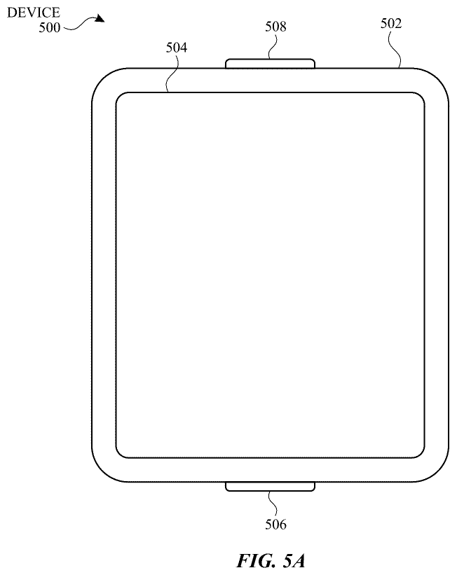

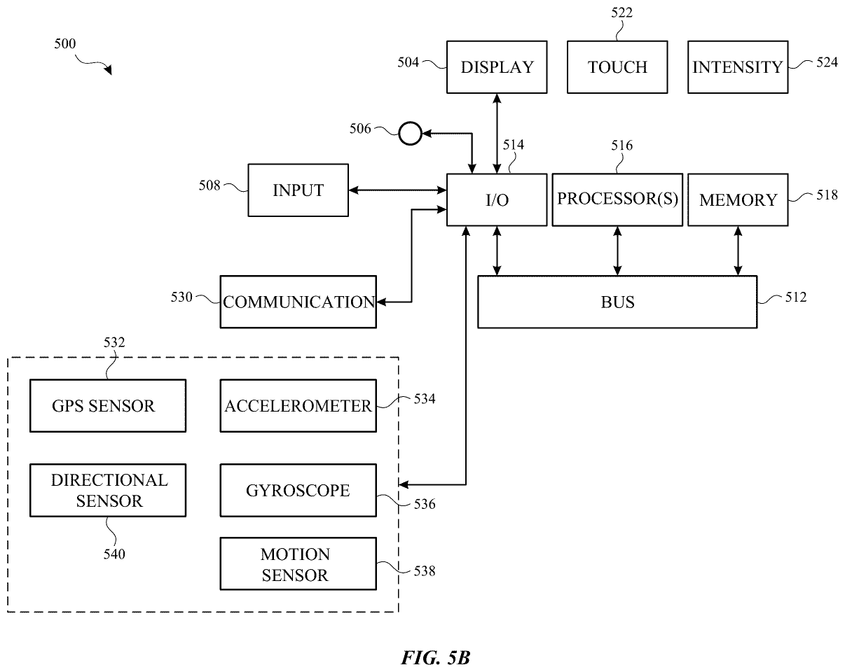

FIG. 5A illustrates exemplary personal electronic device 500. Device 500 includes body 502. In some embodiments, device 500 can include some or all of the features described with respect to devices 100 and 300 (e.g., FIGS. 1A-4B). In some embodiments, device 500 has touch-sensitive display screen 504, hereafter touch screen 504. Alternatively, or in addition to touch screen 504, device 500 has a display and a touch-sensitive surface. As with devices 100 and 300, in some embodiments, touch screen 504 (or the touch-sensitive surface) optionally includes one or more intensity sensors for detecting intensity of contacts (e.g., touches) being applied. The one or more intensity sensors of touch screen 504 (or the touch-sensitive surface) can provide output data that represents the intensity of touches. The user interface of device 500 can respond to touches based on their intensity, meaning that touches of different intensities can invoke different user interface operations on device 500.

Exemplary techniques for detecting and processing touch intensity are found, for example, in related applications: International Patent Application Serial No. PCT/US2013/040061, titled “Device, Method, and Graphical User Interface for Displaying User Interface Objects Corresponding to an Application,” filed May 8, 2013, published as WIPO Publication No. WO/2013/169849, and International Patent Application Serial No. PCT/US2013/069483, titled “Device, Method, and Graphical User Interface for Transitioning Between Touch Input to Display Output Relationships,” filed Nov. 11, 2013, published as WIPO Publication No. WO/2014/105276, each of which is hereby incorporated by reference in their entirety.

In some embodiments, device 500 has one or more input mechanisms 506 and 508. Input mechanisms 506 and 508, if included, can be physical. Examples of physical input mechanisms include push buttons and rotatable mechanisms. In some embodiments, device 500 has one or more attachment mechanisms. Such attachment mechanisms, if included, can permit attachment of device 500 with, for example, hats, eyewear, earrings, necklaces, shirts, jackets, bracelets, watch straps, chains, trousers, belts, shoes, purses, backpacks, and so forth. These attachment mechanisms permit device 500 to be worn by a user.

FIG. 5B depicts exemplary personal electronic device 500. In some embodiments, device 500 can include some or all of the components described with respect to FIGS. 1A, 1B, and 3A. Device 500 has bus 512 that operatively couples I/O section 514 with one or more computer processors 516 and memory 518. I/O section 514 can be connected to display 504, which can have touch-sensitive component 522 and, optionally, intensity sensor 524 (e.g., contact intensity sensor). In addition, I/O section 514 can be connected with communication unit 530 for receiving application and operating system data, using Wi-Fi, Bluetooth, near field communication (NFC), cellular, and/or other wireless communication techniques. Device 500 can include input mechanisms 506 and/or 508. Input mechanism 506 is, optionally, a rotatable input device or a depressible and rotatable input device, for example. Input mechanism 508 is, optionally, a button, in some examples.

Input mechanism 508 is, optionally, a microphone, in some examples. Personal electronic device 500 optionally includes various sensors, such as GPS sensor 532, accelerometer 534, directional sensor 540 (e.g., compass), gyroscope 536, motion sensor 538, and/or a combination thereof, all of which can be operatively connected to I/O section 514.

Memory 518 of personal electronic device 500 can include one or more non-transitory computer-readable storage mediums, for storing computer-executable instructions, which, when executed by one or more computer processors 516, for example, can cause the computer processors to perform the techniques described below, including processes 700-800 and 1000 (FIGS. 7-8 and 10). A computer-readable storage medium can be any medium that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on CD, DVD, or Blu-ray technologies, as well as persistent solid-state memory such as flash, solid-state drives, and the like. Personal electronic device 500 is not limited to the components and configuration of FIG. 5B, but can include other or additional components in multiple configurations.

As used here, the term “affordance” refers to a user-interactive graphical user interface object that is, optionally, displayed on the display screen of devices 100, 300, and/or 500 (FIGS. 1A, 3A, and 5A-5B). For example, an image (e.g., icon), a button, and text (e.g., hyperlink) each optionally constitute an affordance.

As used herein, the term “focus selector” refers to an input element that indicates a current part of a user interface with which a user is interacting. In some implementations that include a cursor or other location marker, the cursor acts as a “focus selector” so that when an input (e.g., a press input) is detected on a touch-sensitive surface (e.g., touchpad 355 in FIG. 3A or touch-sensitive surface 451 in FIG. 4B) while the cursor is over a particular user interface element (e.g., a button, window, slider, or other user interface element), the particular user interface element is adjusted in accordance with the detected input. In some implementations that include a touch screen display (e.g., touch-sensitive display system 112 in FIG. 1A or touch screen 112 in FIG. 4A) that enables direct interaction with user interface elements on the touch screen display, a detected contact on the touch screen acts as a “focus selector” so that when an input (e.g., a press input by the contact) is detected on the touch screen display at a location of a particular user interface element (e.g., a button, window, slider, or other user interface element), the particular user interface element is adjusted in accordance with the detected input. In some implementations, focus is moved from one region of a user interface to another region of the user interface without corresponding movement of a cursor or movement of a contact on a touch screen display (e.g., by using a tab key or arrow keys to move focus from one button to another button); in these implementations, the focus selector moves in accordance with movement of focus between different regions of the user interface. Without regard to the specific form taken by the focus selector, the focus selector is generally the user interface element (or contact on a touch screen display) that is controlled by the user so as to communicate the user's intended interaction with the user interface (e.g., by indicating, to the device, the element of the user interface with which the user is intending to interact). For example, the location of a focus selector (e.g., a cursor, a contact, or a selection box) over a respective button while a press input is detected on the touch-sensitive surface (e.g., a touchpad or touch screen) will indicate that the user is intending to activate the respective button (as opposed to other user interface elements shown on a display of the device).

As used in the specification and claims, the term “characteristic intensity” of a contact refers to a characteristic of the contact based on one or more intensities of the contact. In some embodiments, the characteristic intensity is based on multiple intensity samples. The characteristic intensity is, optionally, based on a predefined number of intensity samples, or a set of intensity samples collected during a predetermined time period (e.g., 0.05, 0.1, 0.2, 0.5, 1, 2, 5, 10 seconds) relative to a predefined event (e.g., after detecting the contact, prior to detecting liftoff of the contact, before or after detecting a start of movement of the contact, prior to detecting an end of the contact, before or after detecting an increase in intensity of the contact, and/or before or after detecting a decrease in intensity of the contact). A characteristic intensity of a contact is, optionally, based on one or more of: a maximum value of the intensities of the contact, a mean value of the intensities of the contact, an average value of the intensities of the contact, a top 10 percentile value of the intensities of the contact, a value at the half maximum of the intensities of the contact, a value at the 90 percent maximum of the intensities of the contact, or the like. In some embodiments, the duration of the contact is used in determining the characteristic intensity (e.g., when the characteristic intensity is an average of the intensity of the contact over time). In some embodiments, the characteristic intensity is compared to a set of one or more intensity thresholds to determine whether an operation has been performed by a user. For example, the set of one or more intensity thresholds optionally includes a first intensity threshold and a second intensity threshold. In this example, a contact with a characteristic intensity that does not exceed the first threshold results in a first operation, a contact with a characteristic intensity that exceeds the first intensity threshold and does not exceed the second intensity threshold results in a second operation, and a contact with a characteristic intensity that exceeds the second threshold results in a third operation. In some embodiments, a comparison between the characteristic intensity and one or more thresholds is used to determine whether or not to perform one or more operations (e.g., whether to perform a respective operation or forgo performing the respective operation), rather than being used to determine whether to perform a first operation or a second operation.

Attention is now directed towards embodiments of user interfaces (“UI”) and associated processes that are implemented on an electronic device, such as portable multifunction device 100, device 300, or device 500.

FIGS. 6A-6BH illustrate exemplary user interfaces for managing hearing health, in accordance with some embodiments. The user interfaces in these figures are used to illustrate the processes described below, including the processes in FIGS. 7 and 8.

FIG. 6A illustrates computer system 600 that includes display 602 and hardware (e.g., wireless, such as Bluetooth, and/or wired) for connecting to earphones (e.g., headphones, headsets, and/or earbuds). At FIG. 6A, computer system 600 is displaying, via display 602, system settings user interface 604 that includes list 604A of paired devices. Notably, earbuds 610 and earbuds 620 are not currently paired with computer system 600 (e.g., using a short-range communication protocol and/or a different protocol), as indicated by list 604A not including indications of earbuds 610 or 620.

At FIG. 6B, computer system 600 detects that earbuds 610 are ready to be paired to computer system 600 and, in response, computer system 600 displays pairing sheet 606A over home screen 606. Computer system 600 detects input 650A (e.g., a tap input and/or a tap-and-hold input) directed to connect option 606B of pairing sheet 606A and, in response, computer system 600 initiates a process to pair (e.g., using a short-range communication protocol and/or a different protocol) earbuds 610 to computer system 600. Earbuds 610 include left earbud 610A and right earbud 610B. Earbuds 610 are configured to be placed and/or stored in case 612. Computer system 600 determines that earbuds 610 are a type (e.g., a second type) of audio device (e.g., that earbuds 610 do not have active noise canceling, do not include a microphone, and/or are not configured to create a seal with the user's ear (e.g., a canal of the user's ear and/or the user's outer ear)). Based on the determination that earbuds 610 are the second type of audio device, computer system 600 proceeds (as part of the process to pair earbuds 610 to computer system 600) to pair earbuds 610 with computer system 600 without providing an option to perform a hearing test. After pairing earbuds 610 with computer system 600 (and without providing an option to perform a hearing test and without performing a hearing test), computer system 600 updates sheet 606A to indicate that earbuds 610 are paired with computer system 600, as shown in FIG. 6C. Accordingly, computer system 600 does not provide an option to perform a hearing test using earbuds 610 during the process to pair earbuds 610 to computer system 600 because earbuds 610 would not provide sufficiently accurate results for the hearing test.

At FIG. 6D, computer system 600 detects that earbuds 620 are ready to be paired to computer system 600 and, in response, computer system 600 displays pairing sheet 608 over home screen 606. Computer system 600 detects input 650B (e.g., a tap input and/or a tap-and-hold input) directed to connect option 608A of pairing sheet 608 and, in response, computer system 600 initiates a process to pair earbuds 620 to computer system 600. Earbuds 620 include left earbud 620A and right earbud 620B. Earbuds 620 are configured to be placed and/or stored in case 622. Computer system 600 determines (e.g., in response to input 650B) that earbuds 620 are a type (e.g., a first type) of audio device (e.g., that earbuds 620 do have active noise canceling, do include a microphone, and/or are configured to create a seal with the user's ear (e.g., a canal of the user's ear and/or the user's outer ear)). Based on the determination that earbuds 620 are the first type of audio device, computer system 600 proceeds (as part of the process to pair earbuds 620 to computer system 600) to pair earbuds 620 with computer system 600 and to provide the user with option 608B to perform a hearing test, as shown in FIG. 6E.

At FIG. 6E, computer system 600 displays option 608B to perform a hearing test and option 608C to continue the process to pair earbuds 620 to computer system 600 without performing the hearing test. At FIG. 6E, computer system 600 receives a user input. In response to receiving the user input and in accordance with a determination that the user input (e.g., tap input and/or tap-and-hold input 650C) is directed to option 608C to skip the hearing test, computer system 600 proceeds with the process to pair earbuds 620 with the computer system 600 without performing the hearing test, such as by updating sheet 608 to indicate that earbuds 620 are paired with computer system 600 (e.g., similar to FIG. 6C). In response to receiving the user input and in accordance with a determination that the user input (e.g., tap input and/or tap-and-hold input 650D) is directed to option 608B to perform the hearing test, computer system 600 proceeds with the process to perform the hearing test, as shown in FIGS. 6F-6AH.

At FIG. 6F, computer system 600 displays hearing test user interface 630 with option 630A to proceed and option 630B to import hearing test results from a prior hearing test. At FIG. 6F, computer system 600 receives a user input. In response to receiving the user input and in accordance with a determination that the user input (e.g., tap input and/or tap-and-hold input 650E) is directed to option 630A to proceed with the hearing test, computer system 600 proceeds to the user interface shown in FIG. 6G. In response to receiving the user input and in accordance with a determination that the user input (e.g., tap input or tap-and-hold input 650F) is directed to option 630B to import test results, computer system 600 initiates a process to enable the user to import test results from a prior hearing test. In some embodiments, in response to detecting activation of option 630B, computer system 600 provides options to select results of one or more previously performed hearing tests and to configure earbuds 620 as hearing aids based on the selected result(s). In some embodiments, computer system 600 excludes options for results of previously performed hearing tests that are incomplete and/or incompatible with computer system 600 and/or earbuds 620 while displaying options for results of previously performed hearing tests that are complete and/or are compatible with computer system 600 and/or earbuds 620. Thus, computer system 600 optionally reduces the selectable options for results of previously performed hearing tests for import to be used to configure earbuds 620 as hearing aids.

At FIG. 6G, computer system 600 updates hearing test user interface 630 to indicate that computer system 600 will check the performance of earbuds 620 (e.g., if the performance of earbuds 620 has not been recently checked (e.g., within the last month, 6 months, or year)), thereby providing the user with visual feedback about the process that the computer system will perform. In response to detecting user input (e.g., tap input and/or tap-and-hold input 650F) that is directed to option 630C, computer system 600 initiates a process to check the performance of earbuds 620, as shown in FIGS. 6H-6K. During the process to check the performance of earbuds 620, computer system 600 determines whether the earbuds 620 are placed in their case 622 with the lid of case 622 closed. If earbuds 620 are not in their case 622 with the lid of case 622 closed, computer system 600 displays alert 630D, as shown in FIG. 6H, instructing the user to place earbuds 620 in their case 622 (and, optionally, to close the lid) (e.g., with next option 630E disabled). As shown in FIG. 6I, once computer system 600 detects that earbuds 620 are in their case 622 with the lid of case 622 closed, computer system 600 provides next option 630F, which, when activated via an input (e.g., tap input and/or tap-and-hold input 650G), causes computer system 600 to initiate testing of earbuds 620, as shown in FIG. 6J. At FIG. 6J, computer system 600 indicates (e.g., via indication 630G) that the performance test of earbuds 620 is being performed. During the performance test, one or more speakers of earbuds 620 output audio and one or more microphones of earbuds 620 detect/record the audio. The detected/recorded audio is analyzed (e.g., by computer system 600 and/or by earbuds 620) to determine whether the audio is within a threshold performance. For example, the performance test optionally tests one or more of the following for one or both earbuds 620 (e.g., separately and/or concurrently): volume of the recorded audio, frequencies of the recorded audio, and/or volumes of various frequencies of the recorded audio. If the speakers and microphones are not experiencing problems, the recorded audio falls within the threshold performance (e.g., for the one or more tests) and computer system 600 provides indication 630H (e.g., via display 602) that earbuds 620 are suitable for the hearing test, as shown in FIG. 6K. At FIG. 6K, computer system 600 detects an input (e.g., tap input and/or tap-and-hold input 650H) directed to next option 6301 and proceeds to user interface 630 of FIG. 6M. If the speakers and/or microphones are experiencing problems, the recorded audio may fall outside of the threshold performance and computer system 600 provides indication 630J (e.g., via display 602) that earbuds 620 are not suitable for the hearing test, as shown in FIG. 6L. At FIG. 6L, computer system 600 also provides next option 630K to proceed (e.g., when activated by a user input (e.g., tap input and/or tap-and-hold input 650I)) with and/or to complete the pairing process (e.g., by displaying a user interface similar to FIG. 6C). At FIG. 6L, computer system 600 provides import option 630L to display options for importing (e.g., when activated by a user input (e.g., tap input and/or tap-and-hold input 650J)) audiogram data (e.g., information indicative of the user's hearing for a plurality of frequencies, such as for one or two ears of the user), such as from an application of computer system 600 and/or from a server. In some embodiments, in response to detecting activation of import option 630L, computer system 600 provides options to select results of one or more previously performed hearing tests and to configure earbuds 620 as hearing aids based on the selected result(s). In some embodiments, computer system 600 excludes options for results of previously performed hearing tests that are incomplete and/or incompatible with computer system 600 and/or earbuds 620 while displaying options for results of previously performed hearing tests that are complete and/or are compatible with computer system 600 and/or earbuds 620. Thus, computer system 600 optionally reduces the selectable options for results of previously performed hearing tests for import to be used to configure earbuds 620 as hearing aids. In some embodiments, computer system 600 does not prompt (e.g., 630 and 630C at FIG. 6G) to check the performance of earbuds 620 and/or does not check (e.g., FIG. 6J-6K) the performance of earbuds 620 during the pairing process.

At FIG. 6M, computer system 600 displays information about how the hearing test works, thereby providing the user with details about the hearing test process to be performed. In response to detecting activation of (e.g., tap input and/or tap-and-hold input 650K) next option 630M, computer system 600 proceeds to updating user interface 630, as shown in FIG. 6N. At FIG. 6N, computer system 600 collects information about the user of computer system 600 and earbuds 620. Computer system 600 prompts 630N whether the user is 21 years or older, prompts 630O whether the user is currently experiencing some symptoms, and prompts 630P whether the user has been in a loud environment in the last 24 hours (and/or other time period). Computer system 600 detects inputs 650L-650N corresponding to prompts 630N-630P, then detects a user input (e.g., tap input and/or tap-and-hold input 6500) directed to continue option 630Q and, in response, proceeds to updating user interface 630, as shown in FIG. 6O. In some embodiments, the answers to prompts 630N-630P determine whether computer system 600 proceeds with the hearing test (e.g., computer system 600 does not proceed with the hearing test if the user indicates that they have recently been in a loud environment).

At FIG. 6O, computer system 600 prompts 630R the user to find a quiet place to perform the hearing test. In response to detecting activation of (e.g., tap input and/or tap-and-hold input 650P) next option 630S, computer system 600 proceeds to updating user interface 630, as shown in FIG. 6P. At FIG. 6P, computer system 600 prompts 630T the user to place the left and right earbuds of earbuds 620 into the user's ears. In response to detecting activation of (e.g., tap input and/or tap-and-hold input 650Q) next option 630U (and/or in response to detecting that the earbuds 620 have been placed into the user's ears), computer system 600 proceeds to updating user interface 630, as shown in FIG. 6Q.

At FIG. 6Q, computer system 600 displays information about performing a fit check (checking that earbuds 620 fit properly into the user's ears) and a background noise check (that the physical environment that the user is in is sufficiently quiet). In some embodiments, the fit check and the background noise check are performed concurrently, as some of the procedures of the two tests overlap. In some embodiments, the fit check and the background noise check are performed consecutively. At FIG. 6Q, in response to detecting activation of (e.g., tap input and/or tap-and-hold input 650R) start option 630V, computer system 600 proceeds to beginning the fit check and the background noise check and updating user interface 630, as shown in FIG. 6R.

In some embodiments, at FIG. 6R, during the fit check and the background noise check, earbuds 620 (e.g., individually or together) output audio via a speaker of earbuds 620 and a microphone of earbuds 620 is used to concurrently record and/or analyze detected audio. In some embodiments, when the audio output via the speaker of earbuds 620 is detected above a threshold level using the microphone of earbuds 620, computer system 600 determines that the fit of earbuds 620 is improper (e.g., earbuds 620 have not created a sufficient seal with the user's ears to contain some/all audio output by the speaker of earbuds 620) and prompts the user to adjust earbuds 620, as shown in FIG. 6S. When the audio output via the speaker of earbuds 620 is not detected using the microphone of earbuds 620 or is detected below the threshold level, computer system 600 determines that the fit of earbuds 620 is proper (e.g., earbuds 620 have created a sufficient seal with the user's ears to contain most/all audio output by the speaker of earbuds 620) and indicates to the user that earbuds 620 fit well, as shown in FIG. 6T.

Computer system 600 also determines whether the loudness of the physical environment in which the user (and therefore earbuds 620) are located is sufficiently quiet for performing a hearing test via the background noise check. As shown in FIGS. 6U1-6V2, computer system 600 displays environment noise indication 630X of the level (e.g., loudness) (and, optionally, frequency) of audio being detected by earbuds 620. Environment noise indication 630X changes (e.g., in height, in shape, and/or in color) in conjunction with the audio detected by earbuds 620, providing the user with real-time or near real-time visual feedback about the environment's noise levels. This enables the user to change their environment (e.g., move to a different room, turn off a television or other device, and/or close a door) and watch how the change affects the level of environmental noise being detected by earbuds 620. The visual feedback provided by environment noise indication 630X is particularly helpful to individuals that have some hearing loss and cannot properly gauge how loud their environment is on their own. The visual feedback provided by environment noise indication 630X is also particularly helpful because earbuds 620 are (e.g., optionally) actively suppressing environmental noise (e.g., via ANC and/or via physically blocking the user's ear canal), thereby limiting the user's ability to otherwise gauge how loud the environment is. When the level of environmental noise detected by the microphone of earbuds 620 is above a noise threshold, computer system 600 determines, while earbuds 620 are worn by the user, that the environment is too loud for performing the hearing test, as shown in FIG. 6U1. At FIG. 6U1, environment noise indication 630X is displayed with a first characteristics (e.g., in a first color (e.g., yellow and/or red) and/or with a first amount of emphasis) to indicate that the environment is too loud and the height of the bars (e.g., the tallest bar and/or the average height of the bars) optionally corresponds to the current level of environmental noise. In some embodiments, option 632A is displayed, enabling the user (via activation (e.g., tap input or tap-and-hold input 650S) of option 632A) to check the level of environmental noise again. In some embodiments, computer system 600 continually and/or repeatedly updates environment noise indication 630X based on currently detected environmental noise as the user changes their environment (and, optionally, option 632A is not displayed).

FIGS. 6U2-6U4 illustrate an additional example of environment noise indication 630X displayed as part of user interface 630 in preparation for the hearing test. In some embodiments, computer system 600 displays user interface 630 of FIGS. 6U2-6U4 in response to detecting activation of (e.g., tap input and/or tap-and-hold input 650R) start option 630V at FIG. 6Q (e.g., when computer system 600 determines that the fit of earbuds 620 is proper and, therefore, does not display the user interface of FIG. 6S) and/or in response to selection of the check again option 630Z of FIG. 6S (e.g., after the user has adjusted earbuds 620 for proper fit). At FIGS. 6U2-6U4, environment noise indication 630X provides visual feedback (e.g., in real-time or near-real-time) about the current level of environmental noise. As shown in FIGS. 6U2-6U4, environment noise indication 630X visually changes as (e.g., in conjunction with) the current environmental noise changes. At FIG. 6U2, environment noise indication 630X comprises a plurality of vertical bars, including bars 630X1-630X3. In some embodiments, the shape (e.g., width and/or height) of the bars change based on changes in the current environmental noise. In some embodiments, respective bars of environment noise indication 630X correspond to respective ranges of frequencies of environmental noise and a respective bar visually changes (e.g., shape, color, and/or height) when the environmental noise for the respective range of frequencies changes. For example, bar 630X1 corresponds to a range of frequencies centered at 20 Hz, bar 630X2 corresponds to a range of frequencies centered at 32 Hz, bar 630X3 corresponds to a range of frequencies centered at 64 Hz, and other bars correspond to ranges of frequencies centered at 125 Hz, 250 Hz, 500 Hz, 1 kHz, 2 kHz, etc. For example, the height of bar 630X1 changes as the level of environmental noise at (or near) 20 Hz changes (e.g., bar 630X1 gets taller as noise gets louder and/or bar 630X1 gets shorter as noise gets quieter), as seen in FIGS. 6U2-6U4. For another example, the height of bar 630X2 changes as the level of environmental noise at (or near) 32 Hz changes (e.g., bar 630X2 gets taller as noise gets louder and/or bar 630X2 gets shorter as noise gets quieter). Throughout FIGS. 6U2-6U4, environment noise indication 630X is displayed with the first characteristic (e.g., in the first color (e.g., yellow and/or red) and/or the first amount of emphasis) to indicate that the level of environmental noise is above the noise threshold, and therefore too loud to start the hearing test. While the level of environmental noise detected by the microphone of earbuds 620 is above the noise threshold, computer system 600 optionally continues to display message 630AA indicating that the environmental noise level is above the noise threshold. In some embodiments, while the level of environmental noise detected by the microphone of earbuds 620 is above the noise threshold, computer system 600 grays out and/or disables next option 630Y, as shown in FIGS. 6U2-6U4, thereby indicating that the process cannot proceed until the level of environmental noise is reduced to below the noise threshold.

In some embodiments, computer system 600 determines that the level of environmental noise is above or below the noise threshold based on the average environmental audio level across the various frequencies being above or below a threshold level. In some embodiments, computer system 600 determines that the level of environmental noise is above or below the noise threshold based on a highest environmental audio level across the various frequencies being above or below a threshold level.

At FIGS. 6V1-6V2, environment noise indication 630X is displayed with a second characteristics (e.g., in a second color (e.g., green and/or blue) and/or a second amount of emphasis), different from the first characteristic (e.g., the first color and/or the first amount of emphasis), to indicate that the level of environmental noise is below the noise threshold, and therefore the environment is sufficiently quiet to proceed with the process. As shown in FIGS. 6V1-6V2, environment noise indication 630X visually changes as (e.g., in conjunction with) the current environmental noise changes. The height of the bars (e.g., the tallest bar and/or the average height of the bars) of environment noise indication 630X optionally correspond to the current level of environmental noise and change as the current level of environmental noise changes. As shown in FIGS. 6V1-6V2, computer system 600 also optionally displays message 630AB indicating that the environmental noise level is acceptable and/or below the noise threshold. In some embodiments, while computer system 600 displays message 630AB indicating that the environmental noise level is below the noise threshold, computer system detects that the environmental noise level has increased to above the noise threshold (e.g., and remained above the noise threshold for a threshold duration of time) and, in response, computer system 600 transitions back to the user interfaces illustrated with respect to FIGS. 6U2-6U4, including displaying environment noise indication 630X with the first characteristic (e.g., in the first color (e.g., red and/or yellow) and/or with the first amount of emphasis). At FIG. 6V2, while the environmental noise level is below the noise threshold, computer system 600 detects an input (e.g., a tap input and/or a tap-and-hold input 650T) directed to next option 632A and, in response, updates user interface 630 as shown in FIG. 6W.

At FIG. 6W, computer system 600 provides the user with instructions 632B for providing inputs for the hearing test. In some embodiments, the computer system designates a specific area (e.g., a portion of a touch-sensitive surface and/or a portion of an area in 3D space) for the user to activate to indicate that the user has heard a tone during the hearing test. In some embodiments, the user can provide input anywhere on the entire touch-sensitive surface (e.g., 602) to indicate that the user has heard a tone during the hearing test. At FIG. 6W, computer system 600 detects an input (e.g., a tap input and/or a tap-and-hold input 650U) directed to next option 632C and updates user interface 630 as shown in FIG. 6X.

At FIG. 6X, computer system 600 displays progress indicator 636, which shows the progress towards completion of the hearing test (e.g., progress towards completing testing for one ear and/or for completing testing for both ears). At FIG. 6X, earbud 620A also outputs audio 634A (e.g., computer-general speech and/or recorded speech) that the hearing test is starting and/or instructions for performing the hearing test (e.g., computer system 600 outputs audio 634A via earbuds 620). Audio 634A provides the user with instructions in the event that the user is not looking at display 602 and/or that the user has closed their eyes to focus on listening to the tones of the hearing test. The computer system automatically proceeds to user interface 630, as shown in FIG. 6Y.

During the hearing test (e.g., as shown in FIGS. 6Y-6Z and 6AC-6AD), computer system 600 outputs different tones at varying frequencies/pitches and at different volumes, pausing after each tone to see if computer system 600 detects a user input (e.g., tap input 650V) indicating that the user has heard the tone. As the hearing test proceeds, computer system 600 updates progress indicator 636 to show progress towards completion of the hearing test. For example, at FIG. 6Y, earbud 620A outputs tone 634B with a first frequency/tone and, at FIG. 6Z, computer system 600 detects tap input 650V indicating that the user heard tone 634B.

In some embodiments, during the hearing test the environmental noise (e.g., the noise in the room the user is in) increases. When the level of environmental noise detected by the microphone of earbuds 620 exceeds a noise threshold, computer system 600 determines that the environment is too loud for continuing the hearing test and pauses (or ends) the hearing test, as shown in FIGS. 6AA1-6AA4. As described in greater detail above, environment noise indication 630X changes (e.g., in height, in shape, and/or in color) in conjunction with the environmental audio detected by earbuds 620, providing the user with real-time or near real-time visual feedback about the environment's noise levels. This indication suggests or encourages the user to change their environment (e.g., move to a different room, turn off a television or other device, and/or close a door) and watch (in real-time or near real-time) how the change affects the level of environmental noise being detected by earbuds 620. As shown in FIGS. 6AA1-6AA4, environment noise indication 630X visually changes as (e.g., in conjunction with) the current environmental noise changes. Environment noise indication 630X comprises a plurality of vertical bars, including bars 630X1-630X3, as described in greater detail with respect to FIGS. 6U2-6U4. In some embodiments, the shape (e.g., width and/or height) of the bars change based on changes in the current environmental noise. In some embodiments, respective bars of environment noise indication 630X correspond to respective ranges of frequencies of environmental noise and a respective bar visually changes (e.g., shape, color, and/or height) when the environmental noise for the respective range of frequencies changes. For example, bar 630X1 corresponds to a range of frequencies centered at 20 Hz, bar 630X2 corresponds to a range of frequencies centered at 32 Hz, bar 630X3 corresponds to a range of frequencies centered at 64 Hz, and other bars correspond to ranges of frequencies centered at 125 Hz, 250 Hz, 500 Hz, 1 kHz, 2 kHz, etc. For example, the height of bar 630X1 changes as the level of environmental noise at (or near) 20 Hz changes (e.g., bar 630X1 gets taller as noise gets louder and/or bar 630X1 gets shorter as noise gets quieter), as seen in FIGS. 6AA1-6AA4. For another example, the height of bar 630X2 changes as the level of environmental noise at (or near) 32 Hz changes (e.g., bar 630X2 gets taller as noise gets louder and/or bar 630X2 gets shorter as noise gets quieter). Throughout FIGS. 6AA1-6AA2, environment noise indication 630X is displayed with the first characteristic (e.g., in the first color (e.g., yellow and/or red) and/or with the first amount of emphasis) to indicate that the level of environmental noise is above the noise threshold, and therefore too loud to continue the hearing test. At FIGS. 6AA1-6AA2, continue option 632E is optionally disabled (as indicated by being grayed out in FIGS. 6AA1-6AA2) to indicate that the hearing test cannot continue until the environmental noise level is reduced to below the noise threshold. The visual feedback provided by environment noise indication 630X is particularly helpful to those individuals that have some hearing loss and cannot properly gauge how loud their environment is on their own. The visual feedback provided by environment noise indication 630X is also particularly helpful because earbuds 620 are (e.g., optionally) actively suppressing environmental noise (e.g., via ANC and/or via physically blocking the user's ear), thereby limiting the user's ability to otherwise gauge how loud the environment is. At FIG. 6AA1, computer system 600 also optionally outputs (e.g., in response to detecting that the environmental noise level exceeds the noise threshold) audio (e.g., speech) via earbuds 620 indicating that the environmental noise is too high. As shown in FIGS. 6AA1-6AA4, environment noise indication 630X changes (e.g., in height, in shape, and/or in color) in conjunction with changes in the environmental noise. At FIG. 6AA3, the environmental noise falls below the noise threshold (e.g., for a zero or non-zero threshold period of time) and, in response, environment noise indication 630X changes in characteristic (e.g., from the first characteristic to the second characteristic) to indicate that the hearing test can proceed. At FIGS. 6AA3-6AA4, while the level of environmental noise does not exceed the noise threshold, computer system 600 continues to update the shape (e.g., width and/or height) and/or color of the bars based on changes in the current environmental noise. At FIGS. 6AA3-6AA4, while the level of environmental noise does not exceed the noise threshold, continue option 632E is enabled and can be activated by a user to continue the hearing test. In some embodiments, while computer system 600 displays message 630AB indicating that the environmental noise level is below the noise threshold, computer system detects that the environmental noise level has increased to above the noise threshold (e.g., and remained above the noise threshold for a threshold duration of time) and, in response, computer system 600 transitions back to the user interfaces illustrated with respect to FIGS. 6AA1-6AA2, including displaying environment noise indication 630X with the first characteristic (e.g., in the first color (e.g., red and/or yellow) and/or with the first level of emphasis). At FIG. 6AA4, computer system 600 detects an input. In response to detecting the input and in accordance with a determination that the input (e.g., a tap input and/or a tap-and-hold input 650W) is directed to continue option 632E, computer system 600 continues with the hearing test, as shown in FIG. 6AC. In response to detecting the input and in accordance with a determination that the input (e.g., a tap input and/or a tap-and-hold input 650W2) is directed to end option 632E, computer system 600 ends the hearing test (e.g., without continuing the hearing test).

In some embodiments, one or both earbuds 620 are removed from the user's ears during the hearing test. In response to detecting that one or both earbuds 620 are removed from the user's ears, computer system 600 pauses the hearing test, as shown in FIG. 6AB. In some embodiments, computer system 600 ends the hearing test and the test can be restarted from the beginning. In some embodiments, as shown in FIG. 6AB, computer system 600 detects an input (e.g., a tap input and/or a tap-and-hold input 650X) directed to continue option 632F and, in response, continues with the hearing test, as shown in FIG. 6AC.

As the hearing test proceeds, computer system 600 updates progress indicator 636 to show progress towards completion of the hearing test. For example, at FIG. 6AC, earbud 620A outputs tone 634C with a second frequency/tone that is different from the first frequency/tone and, at FIG. 6AD, computer system 600 detects tap input 650Y indicating that the user heard tone 634C. In some embodiments, earbud 620A outputs numerous (e.g., 5, 10, 15, or 20) tones and computer system 600 monitors to detect user inputs indicating the user heard each tone. At FIG. 6AE, the hearing test is completed for the user's left ear. Computer system 600 outputs, via earbuds 620, audio 634D (e.g., computer-generated speech and/or recorded speech) indicating that the test is complete for the left ear. Audio 634D is particularly helpful when the user has their eyes closed and is still focusing on listening for the tones of the hearing test. Subsequently, computer system 600 performs a corresponding test for the other ear of the user, using earbud 620B. At FIG. 6AG (after outputting numerous tones and monitoring for corresponding user input), computer system 600 outputs, via earbuds 620, audio 634E (e.g., speech) indicating that the test is complete for the right ear and proceeds to user interface 630, as shown in FIG. 6AH.

At FIG. 6AH, computer system 600 outputs, via earbuds 620, audio 634F (e.g., speech) indicating that the test is complete. At FIG. 6AH, computer system 600 detects an input (e.g., tap input and/or tap-and-hold input 650Z) directed to results option 632G and, in response, displays results of the hearing test, as shown in FIGS. 6AI and 6AJ. FIGS. 6AI and 6AJ illustrate two exemplary test results 660. At FIG. 6AI, computer system 600 indicates the amount of hearing loss for the left 660A ear and right 660B ear, provides an option to view details results, and gives primary option 660D to not set up earbuds 620 as hearing aids (e.g., based on the user only have mild hearing loss). At FIG. 6AI, computer system 600 detects an input. In response to the input (e.g., 652A) being directed to option 660D, computer system 600 proceeds with the process to pair earbuds 620 with computer system 600 without configuring earbuds 620 to operate as hearing aids using the results of the hearing test. In response to the input (e.g., 652B) being directed to the secondary option 660E, computer system 600 proceeds with the process to pair earbuds 620 with computer system 600, including configuring earbuds 620 to operate as hearing aids using the results of the hearing test and proceeds to user interface 630, as shown in FIG. 6AK. At FIG. 6AJ, computer system 600 indicates that the user has moderate hearing loss in both ears and, as a result, provides primary option 660E to configure (e.g., in response to input 652B) earbuds 620 to operate as hearing aids using the results of the hearing test and proceeds to displaying user interface 630 as shown in FIG. 6AK.

At FIGS. 6AK-6AL, computer system 600 provides additional information to the user via user interface 630, before proceeding to user interface 630 at FIG. 6AM. At FIG. 6AM, computer system 600 indicates that earbuds 620 are configured to operate as hearing aids using the results of the hearing test and provides option 632J to turn on the operation of the hearing aids. In some embodiments, computer system 600 provides earbuds 620 with audiogram data (e.g., separate audiogram data for each ear) that earbuds 620 can use to modify audio to accommodate the user's hearing loss. At FIG. 6AM, in response to detecting input 652E (e.g., a tap input and/or a tap-and-hold input), computer system 600 transmits data to earbuds 620 to turn on the hearing aids feature that uses the results of the hearing test and proceeds to displaying user interface 630, as shown in FIG. 6AN. At FIG. 6AN, computer system 600 indicates that the pairing process and the hearing test are complete and that the hearing aid feature of earbuds 620 has been turned on.

At FIG. 6AO, earbuds 620 detect, using one or more microphones of earbuds 620, environmental audio 642A (e.g., someone speaking in the same room as the user), and output, via one or more speakers of earbuds 620, a modified version 640A of environmental audio 642A. Modified version 640A of the audio is based on the results of the hearing test (e.g., based on audiogram data generated using the results of the hearing test and transmitted by computer system 600 to earbuds 620). In some embodiments, earbuds 620 modify (e.g., using the audiogram data) the audio independent of computer system 600 (e.g., even when computer system 600 is turned off or unavailable), as shown in FIG. 6AO. For example, earbuds 620 stores and uses the audiogram data to modify detected audio, then outputs the modified audio without relying on computer system 600. In some embodiments, earbuds 620 use a first set of audio characteristics that are based on the hearing test results to modify environmental audio.

At FIG. 6AP, computer system 600 is participating in a call (e.g., an audio call via a telephone application), as shown in user interface 662A. Computer system 600 transmits (e.g., via Bluetooth and/or a short-range communication protocol) the audio received as part of the call to earbuds 620 for output via one or more speakers of earbuds 620. Earbuds 620 receive the audio. For example, earbuds 620 stores and uses the audiogram data to modify the received audio, then outputs modified audio 640B via one or more speakers of earbuds 620. In some embodiments, earbuds 620 use a second set of audio characteristics (different from the first set of audio characteristics) that are based on the test results to modify the audio that was received via Bluetooth and/or the short-range communication protocol (e.g., based on the audio being identified as audio of a call (e.g., an audio call and/or video call)). In some embodiments, computer system 600 modifies the audio using the second set of audio characteristics before transmitting the audio to earbuds 620.

At FIG. 6AQ, computer system 600 is playing media (e.g., playing music via a music player application), as shown in user interface 662B. Computer system 600 transmits (e.g., via Bluetooth and/or a short-range communication protocol) the audio of the media to earbuds 620 for output via one or more speakers of earbuds 620. Earbuds 620 receive the audio. For example, earbuds 620 stores and uses the audiogram data to modify the received audio, then outputs modified audio 640C via one or more speakers of earbuds 620. In some embodiments, earbuds 620 use a third set of audio characteristics (different from the first set of audio characteristics and the second set of audio characteristics) that are based on the test results to modify the audio that was received via Bluetooth and/or the short-range communication protocol (e.g., based on the audio being identified as audio of a media application (e.g., a music player and/or a video player)). In some embodiments, computer system 600 modifies the audio using the third set of audio characteristics before transmitting the audio to earbuds 620.

FIG. 6AR illustrates settings user interface 670 for modifying settings of earbuds 620. Option 670A enables and disables the ability to change the volume of earbuds 620 using volume buttons of computer system 600. Activation of hearing aid option 670B (e.g., via input 652G) causes computer system 600 to display hearing aid user interface 672, as shown in FIG. 6AS. Activation of test results option 670C (e.g., via input 652H) causes computer system 600 to display hearing test results, as illustrated in FIG. 6BC.

At FIG. 6AS, computer system 600 displays hearing aid user interface 672, which includes option 672A to enable and disable (e.g., via input 652I) the hearing aid option (e.g., to enable/disable earbuds 620 use of the hearing test results to modify audio (e.g., for environmental audio, for calls, and for media playback together)). Option 672E, when activated (e.g., via input 652M) starts another hearing test (e.g., as in FIGS. 6M-6AH), thereby enabling the user to retake (or take) a hearing test as desired. Environment audio option 672B, when activated (e.g., via input 652J), causes display of environment audio adjustment user interface 674, as shown in FIG. 6AT. Media audio option 672C, when activated (e.g., via input 652K), causes display of media audio adjustment user interface 676, as shown in FIG. 6AV. Call audio option 672D, when activated (e.g., via input 652L), causes display of media audio adjustment user interface 678, as shown in FIG. 6AX. Option 672F enables/disables volume control via swipe input, as described with respect to FIGS. 6BE-6BF. Option 672G enables/disables charge reminders, as described with respect to FIG. 6BH.

At FIG. 6AT, computer system 600 displays environment audio adjustment user interface 674, which includes option 674G to enable and disable (e.g., via a tap and/or swipe input) the hearing aid option (e.g., to enable/disable earbuds 620 use of the hearing test results to modify audio) for environmental audio (e.g., audio detected via one or more microphones of earbuds 620) (e.g., without enabling/disabling the hearing aid option for calls and for media playback). Adjustments 674A-674C enable the user to adjust the characteristics (of the first set of audio characteristics) used to modify the audio for environmental audio (e.g., without affecting the characteristics for calls and for media playback). For example, computer system 600 detects swipe input 652N directed to amplification adjustment 674A and, in response, modifies (e.g., increases amplification, based on the magnitude and direction of the input) the amplification of environmental audio. For another example, computer system 600 detects swipe input 652O directed to tone adjustment 674B and, in response, modifies (e.g., increases pitch, based on the magnitude and direction of the input) the pitch of environmental audio. For another example, computer system 600 detects swipe input 652P directed to balance adjustment 674C and, in response, modifies (e.g., shifts left, based on the magnitude and direction of the input) the balance of environmental audio. In some embodiments, when computer system 600 detects a requested change (e.g., 652N-652P) in the characteristics for modifying environmental audio, computer system 600 transmits data (e.g., updated audiogram data) based on the requested change to earbuds 620 such that earbuds 620 can modify the environmental audio (in view of the requested change). FIG. 6AU illustrates the result of inputs 652N-652P. As shown in FIG. 6AU, computer system 600 displays an indication (e.g., 674D-674F) of the last location (and thus, value) of each adjustment (e.g., 674A-674C), thereby allowing the user to revert an adjustment change easily and accurately.

Similarly, at FIG. 6AV, computer system 600 displays media audio adjustment user interface 676, which includes option 676G to enable and disable (e.g., via a tap and/or swipe input) the hearing aid option (e.g., to enable/disable earbuds 620 use of the hearing test results to modify audio) for media audio (e.g., audio output by a media application of computer system 600 and transmitted to earbuds 620) (e.g., without enabling/disabling the hearing aid option for calls and for environmental audio). Adjustments 676A-676C enable the user to adjust the characteristics used to modify the audio for media audio (e.g., without affecting the characteristics for calls and for environmental audio). For example, computer system 600 detects swipe input 652Q directed to amplification adjustment 676A and, in response, modifies (e.g., decreases amplification, based on the magnitude and direction of the input) the amplification of media audio. For another example, computer system 600 detects swipe input 652R directed to tone adjustment 676B and, in response, modifies (e.g., increases pitch, based on the magnitude and direction of the input) the pitch of media audio. For another example, computer system 600 detects swipe input 652S directed to balance adjustment 676C and, in response, modifies (e.g., shifts right, based on the magnitude and direction of the input) the balance of media audio. In some embodiments, when computer system 600 detects a requested change (e.g., 652Q-652S) in the characteristics for modifying media audio, computer system 600 transmits data (e.g., updated audiogram data) based on the requested change to earbuds 620 such that earbuds 620 can modify the media audio (in view of the requested change). FIG. 6AW illustrates the result of inputs 652Q-652S. As shown in FIG. 6AW, computer system 600 displays an indication (e.g., 676D-676F) of the last location (and thus, value) of each adjustment (e.g., 676A-676C), thereby allowing the user to revert an adjustment change easily and accurately.

Similarly, at FIG. 6AX, computer system 600 displays call audio adjustment user interface 678, which includes option 678G to enable and disable (e.g., via a tap and/or swipe input) the hearing aid option (e.g., to enable/disable earbuds 620 use of the hearing test results to modify audio) for call audio (e.g., audio output by an audio call or video call application of computer system 600 and transmitted to earbuds 620) (e.g., without enabling/disabling the hearing aid option for media playback and for environmental audio). Adjustments 678A-678C enable the user to adjust the characteristics used to modify the audio for call audio (e.g., without affecting the characteristics for media playback and for environmental audio). For example, computer system 600 detects swipe input 652T directed to amplification adjustment 678A and, in response, modifies (e.g., increases amplification, based on the magnitude and direction of the input) the amplification of call audio. For another example, computer system 600 detects swipe input 652U directed to tone adjustment 678B and, in response, modifies (e.g., decreases pitch, based on the magnitude and direction of the input) the pitch of call audio. For another example, computer system 600 detects swipe input 652V directed to balance adjustment 678C and, in response, modifies (e.g., shifts left, based on the magnitude and direction of the input) the balance of call audio. In some embodiments, when computer system 600 detects a requested change (e.g., 652T-652V) in the characteristics for modifying call audio, computer system 600 transmits data (e.g., updated audiogram data) based on the requested change to earbuds 620 such that earbuds 620 can modify call audio (in view of the requested change). FIG. 6AY illustrates the result of inputs 652T-652V. As shown in FIG. 6AY, computer system 600 displays an indication (e.g., 678D-678F) of the last location (and thus, value) of each adjustment (e.g., 678A-678C), thereby allowing the user to revert an adjustment change easily and accurately.

In some embodiments, Hearing Loss Compensation (HLC) is a hearing loss compensation algorithm. HLC gain can be adjusted in a limited range by the user through the fine-tuning settings (e.g., as shown in FIGS. 6AT-6AY), which include amplification (e.g., 674A, 676A, and/or 678A), tone (e.g., 674B, 676B, and/or 678B), and balance (e.g., 674C, 676C, and/or 678C) sliders. The user can adjust the amplification slider (e.g., 674A, 676A, and/or 678A) to add gain within the range of −6 dB to +6 dB. The balance slider (e.g., 674C, 676C, and/or 678C) adds up to 3 dB amplification to the right bud and −3 dB to the left bud simultaneously, when the slider is moved to the right, (and vice-versa). This leads to a maximum of 6 dB inter-aural level difference. The tone slider (e.g., 674B, 676B, and/or 678B) makes equal and opposite adjustments above and below 1500 Hz. Pushing the tone slider (e.g., 674B, 676B, and/or 678B) to the right, more gain is applied above 1500 Hz and less gain below 1500 Hz in equal amounts (and vice versa) with a maximum change of 4.5 dB. HLC gain provides maximum of approximately 25 dB and minimum of 0 dB.

FIGS. 6AZ-6BB illustrate using earbuds 620 after the audio modifications have been adjusted in FIGS. 6AT-6AY. At FIG. 6AZ, earbuds 620 use the adjusted first set of audio characteristics (e.g., received from computer system 600 based on the user adjustments at FIG. 6AT) to modify environmental audio. Earbuds 620 detect, using one or more microphones of earbuds 620, environmental audio 642B (e.g., someone speaking in the same room as the user), and output, via one or more speakers of earbuds 620, a modified version 640D of environmental audio 642B. Modified version 640B of the audio is based on the results of the hearing test. In some embodiments, earbuds 620 modify the audio independent of computer system 600 (e.g., even when computer system 600 is turned off or unavailable), as shown in FIG. 6AZ. For example, earbuds 620 stores and uses the audiogram data to modify detected audio 642B, then outputs modified audio 640D without relying on computer system 600. In some embodiments, earbuds 620 use the adjusted first set of audio characteristics that are based on the test results to modify environmental audio 642B. Similarly, at FIG. 6BA, earbuds 620 use the adjusted second set of audio characteristics (e.g., received from computer system 600 based on the user adjustments at FIG. 6AX) to modify call audio 640E. Similarly, at FIG. 6BB, earbuds 620 use the adjusted third set of audio characteristics (e.g., received from computer system 600 based on the user adjustments at FIG. 6AW) to modify media audio 640F (e.g., for media audio received from video application 662C).

FIG. 6BC illustrates exemplary hearing test result 664, including audiogram data 664A for a left ear and 664B for a right ear, and details 664C for a certain frequency based on input 652W. FIG. 6BD illustrates exemplary settings user interface 668, which includes option 668A to enable/disable noise cancellation and enable/disable audio transparency, option 668B to take (or retake) a hearing test, option 668C to turn on/off and modify various types of hearing enhancements, option 668D to turn on/off hearing protection (e.g., reducing loud environmental noises), and option 668E to turn on/off conversation boost (e.g., making voices in the environment louder when the voices are directed to the user). In some embodiments, computer system 600 detects an input (e.g., tap input and/or tap-and-hold input 652X) directed to option 668B and initiates a hearing test (e.g., as shown in FIGS. 6W-6AH). In some embodiments, earbuds 620 can operate as hearing aids while continuing to provide one or more (or all) of the following features: audio transparency (e.g., detecting environment audio and outputting the audio via speakers of earbuds 620), ANC (Active Noise Cancellation), adaptive audio (e.g., including conversation awareness, conversation boost, and personalized volume), loud sound reduction (e.g., using transparency mode), and, optionally, additional features (e.g., audio and non-audio) of earbuds 620.

FIG. 6BE-6BF illustrate a feature that enables earbuds 620 to modify the volume of various types of audio. Earbuds 620 detect a swipe input (e.g., swipe 652Y), such as along a stem of earbud 620A. In response to detecting the swipe input and in accordance with a determination that non-environmental audio (e.g., such as call audio, media audio, and/or audio other than environmental audio) is being currently output by earbuds 620 (e.g., with or without environmental audio), earbuds 620 adjusts (based on the direction and/or magnitude of the swipe input) the volume of the non-environmental audio that is currently being output by earbuds 620 without adjusting the volume of any environmental audio. In response to detecting the swipe input and in accordance with a determination that environmental audio is being currently output by earbuds 620 without outputting non-environmental audio, earbuds 620 adjusts (based on the direction/magnitude of the swipe input) the volume of the environmental audio that is being currently output by earbuds 620 without modifying the volume of any non-environmental audio. For example, at FIG. 6BE input 652Y adjusts the volume of environmental audio because only environmental audio 642C is being output 640G by earbuds 620A when input 652Y is detected, as shown by the increased volume in FIG. 6BF (e.g., for 642D being output as 640H).

FIG. 6BG illustrates home screen 606 of computer system 600 when earbuds 620 are connected and configured to operate as hearing aids. Home screen 606 includes widget 606C that includes visual indication 606D of a volume setting for earbuds 620 and option 606E, which, when activated, causes computer system 600 to display a settings user interface (e.g., any one or more of the user interfaces shown in FIGS. 6AR-6AY) for modifying the amplification of earbuds 620.

FIG. 6BH illustrates earbuds 620 automatically outputting, via one or more speakers of earbuds 620, audio 640I (e.g., computer-general speech and/or recorded speech) that includes battery charge information. In some embodiments, in response to reaching (e.g., dropping to or below) a threshold battery charge value (e.g., 50%, 25%, and or 5% battery remaining), in accordance with a determination that earbuds 620 are operating as hearing aids, earbuds 620 output audio that includes charge information (e.g., amount (e.g., percent and/or time (e.g., in minutes and/or hours)) of charge remaining) and in accordance with a determination that earbuds 620 are not operating as hearing aids, earbuds 620 forgoes outputting the audio that includes charge information. Thus, users that are using earbuds 620 as hearing aids are provided with additional information about the battery charge level of earbuds 620 during their use.

FIG. 7 is a flow diagram illustrating methods of prompting a user to initiate a hearing test, in some embodiments. Method 700 is performed at a computer system (e.g., 100, 300, 500, and/or 600) (e.g., a smart phone, a smart watch, a tablet, a laptop, a desktop, a wearable device, wrist-worn device, and/or head-mounted device) that is in communication with one or more input devices (e.g., a touch-sensitive surface, a touch-sensitive display 602, a button, a rotatable input mechanism, a depressible and rotatable input mechanism, a camera, and/or an accelerometer) (and, optionally, that is in communication with a display generation component (e.g., a display, a touch-sensitive display, and/or a display controller)). Some operations in method 700 are, optionally, combined, the orders of some operations are, optionally, changed, and some operations are, optionally, omitted.

As described below, method 700 provides an intuitive way for initiating a hearing test. The method reduces the cognitive burden on a user for initiating a hearing test, thereby creating a more efficient human-machine interface. For battery-operated computing devices, enabling a user to initiate a hearing test faster and more efficiently conserves power and increases the time between battery charges.

While not paired with a first audio device (e.g., earphones, headphones, a case for earphones and/or headphones), detecting (702), via the one or more input devices (e.g., 602), a request (e.g., 650A and/or 650B) to pair the first audio device (e.g., 610 and/or 620) with the computer system (e.g., 600).

In response to detecting the request (e.g., 650A and/or 650B) to pair the first audio device with the computer system, initiating (704) a process to pair the first audio device (e.g., 610 and/or 620) with the computer system (e.g., 600).

Wherein the process to pair the first audio device (e.g., 610 and/or 620) with the computer system (e.g., 600) includes pairing (706) the first audio device (e.g., 610 and/or 620) with the computer system (e.g., 600) (e.g., via Bluetooth pairing, by registering information about the first audio device with the computer system and registering information about the computer system with the first audio device, and/or by configuring the computer system and first audio device to automatically connect using a wireless protocol); In some embodiments, (e.g., while the first audio device is paired with the computer system and/or before pairing the first audio device with the computer system), the computer system determines whether the first audio device is a first type of audio device or a second type of audio device.

Wherein the process to pair the first audio device (e.g., 610 and/or 620) with the computer system (e.g., 600) includes, in accordance with a determination that the first audio device is a first type of audio device (e.g., 620) (e.g., an audio device that is suitable for a hearing test and/or in-ear earphones with active noise canceling (also referred to as active noise control)), providing (708) (e.g., displaying via a display generation component) an option (e.g., 608) to perform a hearing test (e.g., an audiometry test, a pure tone audiometry test, and/or a word recognition test) that uses the first audio device (e.g., 620) (and, optionally, that uses the computer system).

Wherein the process to pair the first audio device (e.g., 610 and/or 620) with the computer system (e.g., 600) includes, in accordance with a determination that the first audio device is a second type of audio device (e.g., 610) (e.g., an audio device that is suitable for a hearing test and/or in-ear earphones with active noise canceling (also referred to as active noise control)) that is different from the first type of audio device (e.g., 620), forgoing (710) providing the option (e.g., 608B) to perform the hearing test that uses the first audio device. Displaying an option to perform a hearing test when the first audio device is an appropriate audio device provides the user with feedback that a hearing test can be performed. Providing the option to perform the hearing test during the initial pairing process of the first audio device enables the computer system to offer the hearing test at an early opportunity (e.g., before the user otherwise uses the first audio device). In some embodiments, in response to detecting the request to pair the first audio device with the computer system, the computer system initiates a process to pair the first audio device with the computer system, wherein the process includes: pairing the first audio device with the computer system and providing an option to perform the hearing test (e.g., an audiometry test, a pure tone audiometry test, and/or a word recognition test) using the first audio device (e.g., without determining whether the first audio device is a first type of audio device or a second type of audio device and/or displaying the option to perform the hearing test independent from the determination).

In accordance with some embodiments, the process to pair the first audio device (e.g., 620) with the computer system (e.g., 600) includes, in accordance with a determination that the first audio device is a first type of audio device (e.g., 620) (e.g., an audio device that is suitable for a hearing test and/or in-ear earphones with active noise canceling (also referred to as active noise control)), providing (e.g., displaying via a display generation component) an option (e.g., 608C) to not perform the hearing test. In some embodiments, the computer system detects (e.g., while providing the option to perform the hearing test (e.g., an audiometry test, a pure tone audiometry test, and/or a word recognition test) that uses the first audio device), via the one or more input devices, an option selection user input (e.g., 650C and/or 650D) (e.g., selection of the option to perform the hearing test or the option to not perform the hearing test). In response to detecting the option selection user input (e.g., 650C and/or 650D): in accordance with a determination that the option selection user input (e.g., 650D) is directed to the option (e.g., 608B) to perform the hearing test, proceeding with a process to perform the hearing test; and in accordance with a determination that the option selection user input (e.g., 650C) is directed to the option (e.g., 608C) to not perform the hearing test, forgoing proceeding with the process to perform the hearing test (and, optionally, proceeding with the process to pair the first audio device with the computer system, such as by displaying information indicating that the first audio device has been paired with the computer system). Proceeding with the process to perform the hearing test based on user input enables the computer system to adapt to the user's preferences, such as by not spending time to perform the hearing test if the user is already aware that the user does not need a hearing aid, thereby reducing the amount of time to complete the pairing process and reducing the amount of power used by the computer system during the pairing process, which is particularly helpful for battery-powered computer systems.

In accordance with some embodiments, the computer system generates, based on performance of the hearing test, audiogram data (e.g., as used to display audiograms 664A-664B) (e.g., information indicative of the user's hearing for a plurality of frequencies, such as for one or two ears of the user). In some embodiments, the audiogram data is used to generate an audiogram. In some embodiments, the hearing test generates the audiogram. In some embodiments, the hearing information is used to generate the audiogram. In some embodiments, during the hearing test, the computer system determines a first audible threshold for a first frequency and a second audible threshold (different from the first audible threshold) for a second frequency (different from the first frequency). In some embodiments, the hearing test generates a first set of audio characteristics that includes a first hearing level (e.g., a first audio level of hearing (e.g., in dB) for a first frequency of audio) and a second hearing level (e.g., a second audio level of hearing (different from the first level of hearing) (e.g., in dB) for a second frequency of audio (different from the first frequency of audio)). In some embodiments, the hearing test generates (e.g., in addition to the first set of audio characteristics) a second set of audio characteristics that includes a third hearing level (e.g., a third audio level of hearing (e.g., in dB) for the first frequency of audio) and a fourth hearing level (e.g., a fourth audio level of hearing (different from the third level of hearing) (e.g., in dB) for the second frequency of audio (different from the first frequency of audio)) (e.g., wherein the second set of audio characteristics is different from the first set of audio characteristics). The hearing test generating audiogram data enables the computer to use the audiogram data to display an audiogram for the user, enabling the user to see the results of the hearing test, and/or to use the audiogram data to adjust/configure a hearing aid, such as the first audio device, thereby improving the computer system's ability to output audio tailored to the user's hearing needs.

In some embodiments, subsequent to pairing the first audio device (e.g., 620) with the computer system (e.g., 600), performing the hearing test using the first audio device (e.g., 620). In some embodiments, the computer system pairs the first audio device with the computer system before providing the option to perform the hearing test (e.g., an audiometry test, a pure tone audiometry test, and/or a word recognition test) that uses the first audio device. Pairing the first audio device with the computer system before performing the hearing test (e.g., before presenting the option to perform the hearing test) enables the computer system to establish a communication channel (e.g., Bluetooth and/or WiFi) with the first audio device and use that same communication channel for the hearing test, thereby improving the efficiency of the computer system by not needing to employ a temporary communication channel for the hearing test.

In some embodiments, the computer system (e.g., 600) is configured to communicate with one or more display generation components (e.g., 602). Subsequent to performing the process to pair the first audio device (e.g., 620) with the computer system (e.g., 600) (e.g., with or without performing the hearing test) (e.g., while the first audio device is paired with the computer system), the computer system (e.g., 600) displays (e.g., in response to detecting a user request), via the one or more display generation components (e.g., 602), a settings user interface (e.g., 672 in FIG. 6AS, 674-678 in FIGS. 6AT-6AY, and/or 668 in FIG. 6BD) that includes an option (e.g., 672E, “Updating hearing test” in FIGS. 6AT-6AY, and/or 668B) to perform a hearing test using the first audio device (e.g., 620). The computer system detects, via the one or more input devices, an input (e.g., 652M and/or 652X) directed to the option to perform the hearing test using the first audio device (e.g., 620). In response to detecting the input directed to the option to perform the hearing test using the first audio device, the computer system (e.g., 600) proceeds with a process to perform the hearing test (e.g., as shown in FIGS. 6M-6AH). Displaying a settings user interface with an option to perform a hearing test using the first audio device (e.g., the same test as offered during the pairing process) after the process to pair enables the user to perform (and/or re-perform) the hearing test at a later time, without need to unpair and repair the first audio device, thereby improving the man-machine interface.

In some embodiments, the first audio device (e.g., 620) (e.g., earphones and/or headphones) includes one or more speakers (e.g., integrated into the first audio device) and one or more microphones (e.g., integrated into the first audio device). Prior to perform the hearing test, the computer system (e.g., 600) initiates a process to perform an audio input and audio output assessment of the first audio device (e.g., as shown in FIGS. 6G-6L) using the one or more speakers and the one or more microphones of the first audio device (e.g., a check to confirm that the speaker and microphone of the first audio device are both functioning properly). After (e.g., in response to) performing the audio input and audio output assessment of the first audio device: in accordance with a determination that the first audio device passed the audio input and audio output assessment (e.g., volume of audio output by the one or more speakers and detected by the one or more microphones is within a threshold volume range, is within a threshold tone range, and/or exceeds a threshold quality), proceeding (as shown in FIG. 6M) with a process to perform the hearing test that uses the first audio device; and in accordance with a determination that the first audio device did not pass the audio input and audio output assessment, forgoing proceeding with the process to perform the hearing test (and, optionally, displaying a notification (e.g., as shown in FIG. 6L) that the first audio device did not pass the audio input and audio output assessment). Performing the audio input and audio output assessment of the first audio device and proceeding when the first audio devices passes the assessment helps to ensure that the hearing test is performed with hardware that is properly calibrated and results in valid hearing test results, thereby improving the functioning of the computer system and avoiding performing a hearing test that produces invalid results, thereby saving power and avoiding using invalid results for hearing aids.

In some embodiments, the first audio device (e.g., 620) (e.g., earphones and/or headphones) includes one or more microphones (e.g., integrated into the first audio device). During the hearing test, the computer system (e.g., 600) detects, via the one or more microphones of the first audio device (e.g., 620), a level of environmental noise (e.g., noise in the room that the user is in). In response to detecting the level of environmental noise: in accordance with a determination that the level of environmental noise is more than a threshold level (e.g., more than a certain dB), interrupting (e.g., pausing and/or ending) the hearing test (e.g., as in FIGS. 6AA1-6AA3); and in accordance with a determination that the level of environmental noise is less than the threshold level (e.g., less than a certain dB), forgoing interrupting the hearing test (e.g., continuing to proceed with the hearing test). Interrupting the hearing test (e.g., by pausing or ending the test) when environmental noise exceeds a threshold level enables the computer system to allow the user time/opportunity to reduce the environmental noise (e.g., by moving to a different room and/or turning off a source (e.g., tv or radio) of the noise) so that the hearing test will produce valid results (and not produce invalid results), thereby improving the testing process and the man-machine interface.

In response to detecting the level of environmental noise and in accordance with the determination that the level of environmental noise is more than the threshold level (e.g., more than a certain dB), providing (e.g., by displaying, via one or more display generation components) an option (e.g., 632E) to continue the hearing test (e.g., after the level of environmental noise drops below the threshold level). In some embodiments, in response to detecting the level of environmental noise and in accordance with the determination that the level of environmental noise is more than the threshold level, the computer system instructs (e.g., via displaying and/or via an audio output (such as by using the first audio device)) the user to move to a quiet location. In some embodiments, the computer system detects activation of the option to continue the hearing test and, in response, the computer system continues the hearing test. Providing the user with an option to continue the hearing test enables the user to reduce the environmental noise and then finish the hearing test (e.g., by continuing where the hearing test left off and/or restarting the hearing test) and reduces the need for the user to searching through settings to restart the hearing test, thereby reducing the number of inputs required to continue the hearing test.

In some embodiments, the first audio device (e.g., earphones and/or headphones) includes one or more speakers (e.g., integrated into the first audio device) and one or more microphones (e.g., integrated into the first audio device). Subsequent to completing the hearing test, the computer system (e.g., 600) outputs, via the one or more speakers of the first audio device (e.g., 620), first audio (e.g., 640B-640C) using a volume setting that is based on (e.g., equal to) a user-selected volume. Using the first audio device as a hearing aid by outputting audio using the results of the hearing test allows the computer system to mitigate the user's hearing loss, allowing the user to hear sounds better. Outputting audio using the results of the hearing test provides the user with audio feedback about the results of the hearing test (allowing the user to hear whether sounds are better/clearer or not).

In some embodiments, the first audio device (e.g., 620) (e.g., earphones and/or headphones) includes one or more speakers (e.g., integrated into the first audio device). During the hearing test, the computer system (e.g., 600) detects, via the one or more input devices, a volume change user input (e.g., input on a volume button (e.g., up and/or down) and/or displayed object). During the hearing test, in response to detecting the volume change user input, the computer system (e.g., 600) changes, based on the volume change user input, a volume setting (e.g., a system volume setting, an earphone volume setting, an audio playback volume setting) from a first value to a second value that is different from the first value. During the hearing test, while the volume setting is at the second value, outputting, via the one or more speakers, audio that is not based on the second value of the volume setting. Not modifying the volume of audio (e.g., audio used to determine if the user can hear certain sounds at certain volumes) being output during the hearing test when the user changes a system volume setting allows the computer system to control the volume of the audio for the hearing test to produce valid hearing test results.

In some embodiments, the computer system (e.g., 600) changes (e.g., during the hearing test) a volume setting (e.g., a system volume setting, an earphone volume setting, an audio playback volume setting) from a user-selected volume to a system-selected volume (e.g., a predefined volume, a volume that is based on environmental noise, and/or a volume that is based on a type (e.g., make and/or model) of the first audio device). During the (e.g., throughout the entire) hearing test, the computer system (e.g., 600) maintains the volume setting at the system-selected volume. In some embodiments, the computer system detects volume change user inputs and in response to detecting the volume change user inputs: in accordance with a determination that the hearing test is not ongoing (e.g., has not started, is paused, and/or has ended), the computer system changes the volume setting based on the volume change user inputs; and in accordance with a determination that the hearing test is ongoing, the computer system forgoes changing the volume setting based on the volume change user inputs. Not modifying the volume of audio (e.g., audio used to determine if the user can hear certain sounds at certain volumes) being output during the hearing test when the user changes a system volume setting allows the computer system to control the volume of the audio for the hearing test to produce valid hearing test results.

In some embodiments, the first audio device (e.g., 620) (e.g., earphones and/or headphones) includes one or more speakers (e.g., integrated into the first audio device). During the hearing test, the computer system (e.g., 600) outputs, via the one or more speakers of the first audio device (e.g., 620): audio (e.g., 634B) (e.g., a first test tone) with a first volume that is independent of a system volume setting; and audio (e.g., 634C) (e.g., a second test tone) with a second volume, different from the first volume, that is independent of the system volume setting. Outputting audio (e.g., test tones) with volume that is independent of the computer system's volume settings allows the computer system to control the volume of the audio for the hearing test to produce valid hearing test results.

In some embodiments, the first audio device (e.g., 620) (e.g., earphones and/or headphones) includes one or more speakers (e.g., integrated into the first audio device) and one or more microphones (e.g., integrated into the first audio device). The computer system (e.g., 600) performs, by outputting test audio (e.g., 634B-634C) via the one or more speakers of the first audio device (e.g., 620), the hearing test to generate a set of audio characteristics using audiogram data (e.g., information indicative of the user's hearing for a plurality of frequencies, such as for one or two ears of the user). In some embodiments, the audiogram data is used to generate an audiogram. In some embodiments, the hearing test generates the audiogram. The computer system (e.g., 600) receives, via the one or more microphones of the first audio device, audio. The computer system (e.g., 600) outputs, via the one or more speakers of the first audio device, modified audio of the received audio, wherein the modified audio has been adjusted based on the set of audio characteristics. In some embodiments, the computer system (e.g., 600) transmits the set of audio characteristics to the first audio device (e.g., 620) and the first audio device detects the audio via one or more microphones of the first audio device, the first audio device modifies the audio using the set of audio characteristics, and the first audio device outputs the modified audio via the one or more speakers of the first audio device. Performing the hearing test with the first audio device and using the first audio device as a hearing aid that is configured/tuned based on the results of the hearing test enables the computer system to provide the user with audio feedback using the results of the hearing test, which allows the user to understand whether the hearing test was successful and whether the user's ability to hear sounds is improved.

In some embodiments, the first audio device (e.g., 620) (e.g., earphones and/or headphones) includes one or more speakers (e.g., integrated into the first audio device). During the hearing test (e.g., as part of the hearing test), the computer system (e.g., 600) outputs, via the one or more speakers of the first audio device (e.g., 620), first audio (e.g., a first tone at a first frequency and at a first volume). During the hearing test (e.g., as part of the hearing test) and subsequent to outputting the first audio (e.g., 634B and/or 634C), the computer system (e.g., 600) monitors for user input (e.g., 650V and/or 650Y) (e.g., a tap input on a touch sensitive surface of the computer system, activation of a physical button of the computer system, and/or activation of an object displayed by the computer system) indicating that a user of the computer system (e.g., 600) heard the first audio. In some embodiments, the computer system subsequently outputs a second audio by varying the tone and/or the volume and monitoring for second user input indicating that the user heard the second audio. In some embodiments, when the computer system detects the user input within a threshold duration of time after outputting the first audio, the computer system identifies the first audio has having been heard by the user and when the computer system does not detect the user input within the threshold duration of time after outputting the first audio, the computer system identifies the first audio has not having been heard by the user. Outputting audio and monitoring for the user to acknowledge hearing the audio enables the computer system to gauge the user's ability to hear different sounds (e.g., at different frequencies and/or at different volumes) and to use that information to generate the audiogram data and/or an audiogram.

Note that details of the processes described above with respect to method 700 (e.g., FIG. 7) are also applicable in an analogous manner to the methods described below. For example, method 700 optionally includes one or more of the characteristics of the various methods described with reference to methods 800, 1000, and/or 1200. For example, the computer system (e.g., 600) of method 700 is the same or similar to the remote computer system of methods 800 and/or the computer system of 1000 and/or 1200. For another example, the audio device (e.g., 620) of method 700 is the same or similar to the audio device (e.g., 620A) of methods 800, 1000, and 1200. For brevity, these details are not repeated below.

FIG. 8 is a flow diagram illustrating methods of modifying audio characteristics of different types of audio, in some embodiments. Method 800 is performed at a computer system (e.g., 100, 300, 500, 620, and/or 600) that includes a first audio device (e.g., earphones and/or headphones) (e.g., 620, 620A, and/or 620B) with one or more speakers (e.g., integrated into the first audio device) (and, optionally, one or more microphones (e.g., integrated into the first audio device). In some embodiments, the computer system is also in communication with a display generation component (e.g., a display, a touch-sensitive display, and/or a display controller)).

The computer system (e.g., 620) accesses (802) a first set of audio characteristics (e.g., generated using audiogram data (e.g., information indicative of the user's hearing for a plurality of frequencies, such as for one or two ears of the user)) that is based on (e.g., the results of) a hearing test (e.g., that was performed using the computer system and/or imported into the computer system) (e.g., as performed during FIGS. 6F-6AH). In some embodiments, the first set of audio characteristics includes a first hearing level (e.g., a first audio level of hearing (e.g., in dB) for a first frequency of audio) and a second hearing level (e.g., a second audio level of hearing (different from the first level of hearing) (e.g., in dB) for a second frequency of audio (different from the first frequency of audio)).

The computer system (e.g., 620) accesses (804) a second set of audio characteristics (e.g., generated using audiogram data (e.g., information indicative of the user's hearing for a plurality of frequencies, such as for one or two ears of the user)) that is different from the first set of audio characteristics and is based on (e.g., the results of) the hearing test (e.g., that was performed using the computer system and/or imported into the computer system) (e.g., as performed during FIGS. 6F-6AH). In some embodiments, the second set of audio characteristics includes a third hearing level (e.g., a third audio level of hearing (e.g., in dB) for the first frequency of audio) and a fourth hearing level (e.g., a fourth audio level of hearing (different from the third level of hearing) (e.g., in dB) for the second frequency of audio (different from the first frequency of audio)).

The computer system (e.g., 620) receives (806) (e.g., after accessing the first and second set of audio characteristics) (e.g., by detecting (e.g., while the first audio device is being worn by a user of the computer system), via the one or more microphones of the first audio device and/or by receiving from a first application (e.g., a media player application, such as a music player) running on a remote device (e.g., remote computer system 600)) first audio (e.g., audio of someone (other than the user of the computer system) speaking, audio of a conversation, and/or audio of a television).

In response (808) to receiving the first audio and in accordance with a determination that the first audio is a first type of audio (e.g., voice and environment audio detected via microphone of the first audio device), outputting (810), via the one or more speakers of the first audio device, first modified audio (e.g., 640A and/or 640D) of the first audio that has been modified based on the first set of audio characteristics (and, optionally, that has not been modified based on the second set of audio characteristics and has not been modified based on a third set of audio characteristics). In some embodiments, the remote computer system (e.g., 600) modifies the first audio to produce the first modified audio. In some embodiments, the first audio device (e.g., 620 and/or 620A) modifies the first audio to produce the first modified audio.

In response (808) to receiving the first audio and in accordance with a determination that the first audio is a second type of audio (e.g., audio generated by an application running on a remote device (e.g., a phone) and/or the computer system (e.g., a media application, such as a music player application and/or a video player application)) that is different from the first type of audio, outputting (812), via the one or more speakers of the first audio device, second modified audio (e.g., 640C and/or 640F) of the first audio that has been modified based on the second set of audio characteristics (and, optionally, that has not been modified based on the first set of audio characteristics and has not been modified based on the third set of audio characteristics). In some embodiments, the remote computer system (e.g., 600) modifies the first audio to produce the second modified audio. In some embodiments, the first audio device (e.g., 620 and/or 620A) modifies the first audio to produce the second modified audio. The second modified audio is different from the first modified audio. Modify audio using different sets (e.g., first and second) of characteristics based on the type (e.g., source) of the audio enables the computer system to modify the different types of audio in different ways, enabling a user to better hear the various types of audio and/or increasing the quality of each type of audio based on the type of audio, thereby providing an improved man-machine interface.

In some embodiments, the computer system (e.g., 620) accesses a third set of audio characteristics (e.g., generated using audiogram data (e.g., information indicative of the user's hearing for a plurality of frequencies, such as for one or two ears of the user)) that is different from the first set of audio characteristics and the second set of audio characteristics and that is based on (e.g., the results of) the hearing test (e.g., that was performed using the computer system and/or imported into the computer system). In some embodiments, the third set of audio characteristics includes a fifth hearing level (e.g., a fifth audio level of hearing (e.g., in dB) for the first frequency of audio) and a sixth hearing level (e.g., a sixth audio level of hearing (different from the fifth level of hearing) (e.g., in dB) for the second frequency of audio (different from the first frequency of audio)). In response to receiving the first audio and in accordance with a determination that the first audio is a third type of audio (e.g., audio generated by an audio communication application running on the computer system (e.g., a phone application, a video conferencing application, and/or a voice communication application)), the computer system (e.g., 620) outputs, via the one or more speakers of the first audio device, third modified audio (e.g., 640B and/or 640E) of the first audio that has been modified based on the third set of audio characteristics (and, optionally, that has not been adjusted based on the first set of audio characteristics and has not been adjusted based on the second set of audio characteristics). In some embodiments, the remote computer system (e.g., 600) modifies the first audio to produce the third modified audio. In some embodiments, the first audio device (e.g., 620 and/or 620A) modifies the first audio to produce the third modified audio. The third modified audio is different from the first modified audio and the second modified audio. Modify audio using different sets (e.g., first, second, and third) of characteristics based on the type (e.g., source) of the audio enables the computer system to modify the different types of audio in different ways, enabling a user to better hear the various types of audio and/or increasing the quality of each type of audio based on the type of audio, thereby providing an improved man-machine interface.

In some embodiments, the computer system (e.g., 620 and/or 620A) (or, the remote computer system 600) detects (e.g., receiving from a remote device (e.g., 600), such as a mobile phone) input (e.g., based on touch input and/or non-touch input at the remote device) to disable modifying audio of a respective type (e.g., data received from remote computer system 600 based on inputs on 674G, 676G, and/or 678G). In response to detecting the input to disable modifying audio of a respective type: in accordance with a determination that the respective type is the first type of audio, the computer system (e.g., 620 and/or 620A) disables modifying received audio of the first type of audio (e.g., 640D) using the first set of audio characteristics and continues to modify received audio of the second type of audio (e.g., 640F) using the second set of audio characteristics; and in accordance with a determination that the respective type is the second type of audio, the computer system (e.g., 620 and/or 620A) disables modifying received audio of the second type of audio (e.g., 640F) using the second set of audio characteristics and continues to modify received audio of the first type of audio (e.g., 640D) using the first set of audio characteristics. In some embodiments, a user can disable having the computer system automatically modify the first type of audio and/or the second type of audio. Providing the user with an option to disable (and/or enable) automatic modification of the first and/or second type of audio allows the user to select when/whether that type of audio should be modified based on results of the hearing test, thereby improving the user's ability to hear audio and improving the man-machine interface.

In some embodiments, the first set of audio characteristics identifies first amplification values for a first plurality of frequencies (e.g., one value for one frequency, another value for another frequency, and yet another value for an additional frequency) and the second set of audio characteristics identifies second amplification values (e.g., the same as or different from the first amplification values) for a second plurality of frequencies (e.g., one value for one frequency, another value for another frequency, and yet another value for an additional frequency). The first set of audio characteristics and the second set of audio characteristics each identifying amplification values for various frequencies allows the computer system to modify the different types of data using different characteristics (using different sound profiles), thereby improving the quality of the audio output for the user.

In some embodiments, the first amplification values for the first plurality of frequencies includes amplifications values that correspond to a first ear (e.g., right ear of the user) (e.g., for 620B) and amplification values that correspond to a second ear (e.g., left ear of the user) (e.g., for 620A) that is different from the first ear. Thus, in some embodiments, each set of audio characteristics includes different amplification data for different ears of the user. In some embodiments, the second amplification values for the second plurality of frequencies includes amplifications values that correspond to the first ear (e.g., right ear of the user) and amplification values that correspond to the second ear (e.g., left ear of the user). Including different amplification values for different ears enables the computer system to accommodate different types of hearing deficiencies in different ears of the users, thereby improving the hearing of the user.

In some embodiments, the first set of audio characteristics includes a first tone profile (e.g., as modified via 674B at FIGS. 6AT-6AU) and the second set of audio characteristics includes a second tone profile (e.g., as modified via 676B at FIGS. 6AV-6AW), different from the first tone profile. Including different tone profiles for the different sets of audio characteristics enables the computer system to modify different types of audio (e.g., from different sources) using different tone profiles, thereby improving the hearing of the user.

In some embodiments, the first tone profile includes tone information for a first ear (e.g., right ear of the user) and tone information for a second ear that is different from the first ear (e.g., left ear of the user). Thus, in some embodiments, each set of audio characteristics includes tone information for different ears of the user. In some embodiments, the second tone profile includes tone information for the first ear (e.g., right ear of the user) and tone information for the second ear (e.g., left ear of the user). Including different tone information for different ears enables the computer system to accommodate different types of hearing deficiencies in different ears of the users, thereby improving the hearing of the user.

In some embodiments, the first set of audio characteristics includes first audio balance (e.g., left-right balance) information (e.g., as modified via 674C at FIGS. 6AT-6AU) and the second set of audio characteristics includes second audio balance (e.g., left-right balance) information (e.g., as modified via 676C at FIGS. 6AV-6AW) that is different from the first audio balance information. In some embodiments, different sets of audio characteristics include different audio balance between the user's right and left ears. Including balance information in the sets of audio characteristics enables the computer system to accommodate different balance settings for different types of audio, thereby improving the user's ability to hear those types of audio.

In some embodiments, the computer system (e.g., 620 and/or 620A) detects (e.g., receiving from a remote device (e.g., 600), such as a mobile phone) input directed to adjusting (e.g., modifying or changing) the first set of characteristics (e.g., receives data from remote computer system 600 based on inputs 652N-652P). In response to detecting the input directed to adjusting the first set of characteristics, the computer system (e.g., 620 and/or 620A) adjusts the first set of characteristics (e.g., changing a volume, tone, or balance of the first set of characteristics). Subsequent to adjusting the first set of characteristics, the computer system (e.g., 620 and/or 620A) outputs, via the one or more speakers of the first audio device, audio (e.g., 640D) modified using the adjusted first set of characteristics. In some embodiments, the computer system enables the user to independently adjust the first and second set of characteristics. In some embodiments, the input determines how a set of characteristics is adjusted (e.g., the degree to which an adjustment of volume, tone, or balance is made). Enabling the user to modify the first and/or second set of characteristics allows the user to manually change the modifications that the computer system makes to the audio, thereby allowing the user to fine-tune the audio for their preferences and for improved hearing.

In some embodiments, the first audio is audio received using one or more microphones of the first audio device (as in FIGS. 6AO and 6AZ). Modifying, based on the first set of audio characteristics, audio received using microphones of the first audio device enables the computer system to receive audio and provide the user with a live (e.g., real-time or near real-time) modified version of the audio that sounds better to the user.

In some embodiments, the first audio is audio received from an application (e.g., a media application, a music player, a phone application, and/or an audio communication application) running on a remote device (e.g., computer system 600) (e.g., as in FIGS. 6AP-6AQ and 6BA-6BB). Modifying, based on the first set of audio characteristics, audio received from an application enables the computer system to provide the user with a live (e.g., real-time or near real-time) modified version of the audio that sounds better to the user.

Note that details of the processes described above with respect to method 800 (e.g., FIG. 8) are also applicable in an analogous manner to the methods described above and below. For example, method 800 optionally includes one or more of the characteristics of the various methods described with reference to methods 700, 1000, and 1200. For example, the computer system (e.g., 600) of method 700 is the same or similar to the remote computer system of method 800 and/or the computer system of method 1000. For another example, the audio device (e.g., 620) of method 700 is the same or similar to the audio device (e.g., 620A) of method 800 and/or method 1000. For another example, the hearing test in the different processes is the same hearing test. For brevity, these details are not repeated below.

FIGS. 9A-9D illustrate example user interfaces for displaying a visual indication of a current level of environmental noise, in accordance with some embodiments. The user interfaces in these figures are used to illustrate the processes described below, including the processes in FIG. 10.

FIGS. 9A-9D illustrate an example of an environment noise indication and corresponding user interfaces, displayed as environment noise indication 632X as part of user interface 630. In these examples, environment noise indication 632X is displayed during (e.g., in the middle of, and/or after starting and before finishing) a hearing test, but environment noise indication 632X can also be displayed prior to starting the hearing test (e.g., as described with respect to FIGS. 6U1-6U4). In some embodiments, computer system 600 displays user interface 630 of FIG. 9A in response to detecting that the environmental noise level is above a threshold level. At FIGS. 9A-9D, environment noise indication 632X provides visual feedback (e.g., in real-time or near-real-time) about the current level of environmental noise. As shown in FIGS. 9A-9D, environment noise indication 632X visually changes as (e.g., in conjunction with) the current environmental noise changes. In some embodiments, the shape and/or size of environment noise indication 632X changes based on the current environmental noise. In some embodiments, a size of environment noise indication 632X is based on the current environmental noise level. In some embodiments, in accordance with a determination that the environmental noise level is above the threshold level, computer system 600 displays environment noise indication 632X with the first characteristic (e.g., with a first color (e.g., red and/or yellow) and/or a first level of emphasis), as shown in FIGS. 9A-9B. In some embodiments, in accordance with a determination that the environmental noise level is below the threshold level, computer system 600 displays environment noise indication 632X with a second characteristic (e.g., with a second color (e.g., green and/or blue) and/or a second level of emphasis), different from the first characteristic, as shown in FIGS. 9C-9D.

As shown in FIGS. 9A-9B, computer system 600 updates display of environment noise indication 632X based on the current environmental noise level and continue option 632E is optionally disabled (as indicated by being grayed out in FIGS. 9A-9B) to indicate that the hearing test cannot continue until the environmental noise level is reduced to below the noise threshold. At FIG. 9B, while displaying message 630AA indicating that the environmental noise level is above the noise threshold, computer system 600 detects that the environmental noise level has fallen to below the threshold level (e.g., for a threshold duration of time) and, in response, displays the user interface of FIG. 9C. At FIG. 9C, computer system 600 has updated environment noise indication 632X to use the second characteristic, in addition to updating a size and/or shape of environment noise indication 632X. At FIG. 9C, in response to detecting that the environmental noise level is below the threshold level, computer system 600 displays message 630AB indicating that the environmental noise level is acceptable and/or below the noise threshold and continue option 632E is enabled and can be activated by a user to continue the hearing test.

At FIG. 9D, computer system 600 detects an input. In response to detecting the input and in accordance with a determination that the input (e.g., a tap input and/or a tap-and-hold input 650W3) is directed to continue option 632E, computer system 600 continues with the hearing test, as shown (for example) in FIG. 6AC. In response to detecting the input and in accordance with a determination that the input (e.g., a tap input and/or a tap-and-hold input 650W4) is directed to end option 632E2, computer system 600 ends the hearing test (e.g., without continuing the hearing test).

FIG. 10 is a flow diagram illustrating methods of displaying a visual indication of a current level of environmental noise, in accordance with some embodiments. Method 1000 is performed at a computer system (e.g., 100, 300, 500, and/or 600) (e.g., a smart phone, a smart watch, a tablet, a laptop, a desktop, a wearable device, wrist-worn device, and/or head-mounted device) that is in communication with a display generation component (e.g., 602) (e.g., a display, a touch-sensitive display, and/or a display controller) and one or more audio devices (e.g., 620) (e.g., earphones, headphones, and/or a microphone (of the computer system and/or of a remote device) and/or an audio analyzer). Some operations in method 1000 are, optionally, combined, the orders of some operations are, optionally, changed, and some operations are, optionally, omitted.

As described below, method 1000 provides an intuitive way for displaying a visual indication of a current level of environmental noise. The method reduces the cognitive burden on a user for determining the current level of environmental noise, thereby creating a more efficient human-machine interface. For battery-operated computing devices, enabling a user to assess the level of environmental noise faster and more efficiently conserves power and increases the time between battery charges.

During a process (1002) to perform (e.g., using the computer system) a hearing test, the computer system (e.g., 600) displays (1004), via the display generation component (e.g., 602), a visual indication (e.g., 630X at FIGS. 6U1-6U4, 6AA1-6AA4 and/or 632X at FIGS. 9A-9D) of a current level of environmental noise, wherein the visual indication visually changes as (e.g., in conjunction with) the current level of environmental noise changes.

During the process (1002) to perform (e.g., using the computer system) the hearing test, while displaying (1004) the visual indication (e.g., 630X at FIGS. 6U1-6U4, 6AA1-6AA4 and/or 632X at FIGS. 9A-9D) of the current level (e.g., loudness) of environmental noise (e.g., noise in the physical environment in which the user and/or computer system is located), the computer system (e.g., 600) detects (1006), via the one or more audio devices (e.g., 620), a change in the current level of environmental noise. In some embodiments, the computer system receives, via the one or more audio devices, information (e.g., audio information, loudness information, and/or audio) about (e.g., that includes and/or indicates a level of) a current environmental noise and updates the visual indication based on the information.

During the process (1002) to perform (e.g., using the computer system) the hearing test, in response to detecting the change in the current level of environmental noise, the computer system (e.g., 600) updates (1009) display (e.g., visually changing a size, shape, location, and/or color), via the display generation component (e.g., 602), of the visual indication (e.g., 630X at FIGS. 6U1-6U4, 6AA1-6AA4 and/or 632X at FIGS. 9A-9D) of the current level of environmental noise to correspond to the current level of environmental noise (e.g., as shown in FIGS. 6U1-6U4, 6AA1-6AA4, and 9A-9D). Displaying a visual indication that changes based on the loudness of the physical environment of the user provides the user with visual feedback of the amount of noise being detected and informs the user as to whether the loudness of the physical environment will negatively affect the user's ability to properly participate in a hearing test, thereby providing improved visual feedback. The visual indication optionally also prompts the user to act, such as by moving to a quieter environment or reducing the noise in the current environment.

In some embodiments, updating display of the visual indication (e.g., 630X at FIGS. 6U1-6U4, 6AA1-6AA4 and/or 632X at FIGS. 9A-9D) of the current level of environmental noise includes changing a shape (e.g., over time and/or in conjunction with the change in the environmental noise) of the visual indication of the current level of environmental noise (e.g., as shown in FIGS. 6U1-6U4, 6AA1-6AA4, and 9A-9D). Displaying a visual indication that changes shape based on the loudness of the physical environment of the user provides the user with visual feedback of the amount of noise being detected and informs the user as to whether the loudness of the physical environment will negatively affect the user's ability to properly participate in a hearing test, thereby providing improved visual feedback.

In some embodiments, the visual indication (e.g., 630X at FIGS. 6U1-6U4 and/or 6AA1-6AA4) of the current level of environmental noise comprises a plurality of bars (e.g., bars 630X1-630X3) (e.g., 5 bars, 15 bars, 17 bars, or 20 bars) arranged in parallel (e.g., vertical bars placed adjacent to each other). Displaying a visual indication that includes a plurality of bars that change based on the loudness of the physical environment of the user provides the user with visual feedback of the amount of noise being detected and informs the user as to whether the loudness of the physical environment will negatively affect the user's ability to properly participate in a hearing test, thereby providing improved visual feedback.

In some embodiments, updating display of the visual indication (e.g., 630X at FIGS. 6U1-6U4 and/or 6AA1-6AA4) of the current level of environmental noise includes changing a height of one or more (e.g., one, two, five, or all) bars (e.g., bars 630X1-630X3) of the plurality of bars (e.g., 630X) based on the change in the current level of environmental noise. In some embodiments, a first bar of the plurality of bars corresponds to a first frequency or a first range of frequencies of the environmental noise and a second bar of the plurality of bars corresponds to a second frequency or a second range of frequencies (different from the first) of the environmental noise. In some embodiments, the height of a respective bar corresponds to a volume of audio in the environment at the corresponding frequency or range of frequencies. Changing the height of different bars of the visual indication provides the user with visual feedback about the loudness of different frequencies, thereby allowing the user to identify the sounds and the sources of the sounds in the user's environment more easily and providing improved visual feedback.

In some embodiments, updating display of the visual indication (e.g., 630X at FIGS. 6U1-6U4 and/or 6AA1-6AA4) of the current level of environmental noise includes changing a height (e.g., over time and/or in conjunction with the change in the environmental noise) of the visual indication (e.g., 630X at FIGS. 6U1-6U4 and/or 6AA1-6AA4) of the current level of environmental noise. In some embodiments, as the environmental volume increases (e.g., for a particular frequency or range of frequencies), a bar (e.g., corresponding to that particular frequency or range of frequencies) of the plurality of bars increases in height. In some embodiments, as the environmental volume decreases (e.g., for a particular frequency or range of frequencies), a bar (e.g., corresponding to that particular frequency or range of frequencies) of the plurality of bars decreases in height. In some embodiments, the height of a respective bar indicates the volume level of environmental noise of a corresponding frequency or range of frequencies. Displaying a visual indication that changes height based on the loudness of the physical environment of the user provides the user with visual feedback of the amount of noise being detected and informs the user as to whether the loudness of the physical environment will negatively affect the user's ability to properly participate in a hearing test, thereby providing improved visual feedback.

In some embodiments, updating display of the visual indication (e.g., 630X at FIGS. 6U1-6U4 and/or 6AA1-6AA4) of the current level of environmental noise includes changing a characteristic (e.g., over time and/or in conjunction with the change in the environmental noise) of the visual indication of the current level of environmental noise (e.g., from a first color at FIG. 6U4 to a second color at FIG. 6V1; from a first color at FIG. 6AA2 to a second color at FIG. 6AA3; and/or from a first color at FIG. 9B to a second color at FIG. 9C). Displaying a visual indication that changes characteristic based on the loudness of the physical environment of the user provides the user with visual feedback of the amount of noise being detected and informs the user as to whether the loudness of the physical environment will negatively affect the user's ability to properly participate in a hearing test, thereby providing improved visual feedback.

In some embodiments, changing a characteristic of the visual indication (e.g., 630X at FIGS. 6U1-6U4 and/or 6AA1-6AA4) of the current level of environmental noise includes: in accordance with a determination that the current level of environmental noise is above a threshold noise level (e.g., the change in the level of noise causes the noise level to rise above the threshold noise level), displaying, via the display generation component (e.g., 602), the visual indication of the current level of environmental noise in a first color (e.g., in yellow or red) (e.g., as in FIGS. 6U1-6U4, 6AA1-6AA2, and/or 9A-9b); and in accordance with a determination that the current level of environmental noise is below the threshold noise level (e.g., the change in the level of noise causes the noise level to drop below the threshold noise level), displaying, via the display generation component (e.g., 602), the visual indication of the current level of environmental noise in a second color (e.g., in green or blue) that is different from the first color (e.g., as in FIGS. 6V1-6V2, 6AA3-6AA4, and/or 9C-9D). Displaying a visual indication that has a first color or second color based on the loudness of the physical environment of the user provides the user with visual feedback of the amount of noise being detected and informs the user as to whether the loudness of the physical environment will negatively affect the user's ability to properly participate in a hearing test, thereby providing improved visual feedback.

In some embodiments, the visual indication of the current level of environmental noise (e.g., that visually changes as the current level of environmental noise changes) is displayed in response to detecting, via the one or more audio devices, that a change in the current level of environmental noise causes the current level of environmental noise to exceed a threshold noise level (e.g., as in FIG. 6AA1 and/or FIG. 9A). Displaying the visual indication of the current level of environmental noise when the environmental noise level exceeds the threshold noise level provides the user with visual feedback that the threshold noise level has been exceeded, thereby providing improved visual feedback.

In some embodiments, while not displaying the visual indication of the current level of environmental noise (e.g., at FIG. 6Z), the computer system (e.g., 600) detects, via the one or more audio devices (e.g., 620), changes in the current level of environmental noise (e.g., at FIG. 6Z). In response to detecting changes in the current level of environmental noise: in accordance with a determination that the current level of environmental noise exceeds (e.g., for a zero or non-zero threshold duration) a threshold noise level, the computer system (e.g., 600) displays, via the display generation component (e.g., 602), the visual indication (e.g., 630X at FIG. 6AA1) of the current level of environmental noise (and, optionally, interrupting an ongoing hearing test); and in accordance with a determination that the current level of environmental noise does not exceed (e.g., for a zero or non-zero threshold duration) the threshold noise level, the computer system (e.g., 600) forgoes display of the visual indication of the current level of environmental noise (and, optionally, forgoing to interrupt the ongoing hearing test) (e.g., as in FIG. 6AC). Displaying the visual indication of the current level of environmental noise when the environmental noise level exceeds the threshold noise level provides the user with visual feedback that the threshold noise level has been exceeded, thereby providing improved visual feedback.

In some embodiments, the visual indication (e.g., 630X and/or 632X) of the current level of environmental noise (e.g., that visually changes as the current level of environmental noise changes) is displayed during a hearing test (e.g., by stopping or pausing the hearing test and displaying the visual indication and/or by displaying the visual indication while the hearing test continues) (e.g., as in FIGS. 6AA1-6AA4 and/or 9A-9C). Displaying the visual indication of the current level of environmental noise during a hearing test provides the user with visual feedback about the environmental noise at a time when monitoring environmental noise is important, thereby providing the user with improved visual feedback.

In some embodiments, the visual indication (e.g., 630X and/or 632X) of the current level of environmental noise (e.g., that visually changes as the current level of environmental noise changes) is displayed in accordance with (e.g., in response to) a determination that the current level of environmental noise exceeds (e.g., for a zero or non-zero threshold duration) a threshold noise level (e.g., at FIG. 6AA1 and/or FIG. 9A). In some embodiments, during the hearing test: in accordance with (and, optionally, in response to) the determination that the current level of environmental noise exceeds (e.g., for a zero or non-zero threshold duration) the threshold noise level, the computer system (e.g., 600) interrupts (e.g., pausing and/or ending) the hearing test; and in accordance with (and, optionally, in response to) a determination that the current level of environmental noise does not exceed (e.g., for a zero or non-zero threshold duration) the threshold noise level, the computer system (e.g., 600) forgoes interrupting (e.g., pausing and/or ending) the hearing test. Automatically interrupting the hearing test when the environmental noise exceeds the threshold noise level prevents the user from performing the hearing test in undesirable noise conditions (which may produce invalid test results) and gives the user an opportunity to remedy the noise conditions without affecting the test results (e.g., without the user missing test tones that they would otherwise hear), thereby improving the man-machine interface and reducing the number of user inputs required to interrupt the hearing test when needed.

In some embodiments, while the hearing test is interrupted (e.g., paused or stopped), the computer system (e.g., 600) detects, via the one or more audio devices (e.g., 620), changes in the current level of environmental noise. In response to detecting changes in the current level of environmental noise: in accordance with a determination that the current level of environmental noise does not exceed (e.g., for a zero or non-zero threshold duration) the threshold noise level, displaying (e.g., concurrently with the visual indication of the current level of environmental noise), via the display generation component (e.g., 602), an option (e.g., displaying a new user interface element and/or updating an existing user interface element to indicate the user interface element is now selectable) to proceed with (e.g., continue and/or restart) the hearing test (e.g., 632E at FIGS. 6AA3-6AA4 and/or FIGS. 9C-9D); and in accordance with a determination that the current level of environmental noise exceeds (e.g., for a zero or non-zero threshold duration) the threshold noise level, the computer system forgoes display (or, optionally, ceasing to display) of the option to proceed with the hearing test (e.g., 632E is grayed out at FIGS. 6AA1-6AA2 and/or FIGS. 9A-9B). In some embodiments, the computer system detects activation of the option to proceed with the hearing test and, in response, proceeds with the hearing test (e.g., ends the pause of the hearing test and/or continues the hearing test at a level of progress toward completion that is based on the amount of progress made before the hearing test was interrupted). Providing the user with an option to proceed with the hearing test when the environmental noise no longer exceeds the threshold noise level provides the user with visual feedback that the loudness of detected environmental noise has reduced and that the test can continue, thereby providing improved visual feedback.

In some embodiments, while the hearing test is interrupted (e.g., paused or stopped), the computer system (e.g., 600) displays (e.g., when the current level of environmental noise exceeds the threshold noise level or independent of the current level of environmental noise), via the display generation component (e.g., 602), an option (e.g., 632E2 at FIGS. 6AA1-6AA4 and/or FIGS. 9A-9D) to end the hearing test. In some embodiments, the computer system detects activation of the option to end the hearing test and, in response, ends the hearing test (e.g., computer system no longer provides an option to continue the same hearing test and/or to perform a hearing test a new hearing test should be started). Providing the user with an option to end the hearing test (e.g., while the environmental noise exceeds the threshold noise level) provides the user quick and easy access to end the test when the environment conditions are not favorable without the need for the user to provide multiple inputs or navigate complicated menus, thereby reducing the number of inputs required to end the hearing test.

In some embodiments, while the hearing test is interrupted (e.g., paused or stopped) and in accordance with (e.g., in response to) a determination that the interruption of the hearing test exceeds a threshold duration (e.g., 30 seconds, 3 minutes, 10 minutes, or 15 minutes) of time (e.g., user isn't able to get to a quiet space and/or the computer system does not detect select of the option to proceed with the hearing test), the computer system (e.g., 600) ends the hearing test (e.g., automatically and without requiring additional user input). In some embodiments, in accordance with a determination that the interruption of the hearing test has not exceeded the threshold duration of time, the computer system forgoes automatically ending the hearing test. Automatically ending the hearing test when the interruption of the hearing test exceeds the threshold duration of time reduces the number of inputs required from the user and prevents the test from proceeding and producing potentially invalid test results, thereby improving the man-machine interface.

In some embodiments, the visual indication (e.g., 630X) of the current level of environmental noise (e.g., that visually changes as the current level of environmental noise changes) is displayed prior to starting a hearing test (e.g., in response to the computer system detecting that the current level of environmental noise exceeds threshold noise level) (e.g., at FIGS. 6U1-6U4). Displaying a visual indication that changes based on the loudness of the physical environment of the user provides the user with visual feedback of the amount of noise being detected before the user starts the test and informs the user as to whether the loudness of the physical environment is appropriate for starting the test, thereby providing improved visual feedback.

In some embodiments, while displaying the visual indication (e.g., 630X) of the current level of environmental noise, the computer system (e.g., 600) detects, via the one or more audio devices (e.g., 620), changes in the current level of environmental noise. In response to detecting changes in the current level of environmental noise: in accordance with a determination that the current level of environmental noise does not exceed (e.g., for a zero or non-zero threshold duration) a threshold noise level, the computer system (e.g., 600) displays, via the display generation component (e.g., 602), an option (e.g., 632A at FIGS. 6V1-6V2) (e.g., displaying a new user interface element and/or updating an existing user interface element to indicate the user interface element is now selectable) to proceed with (e.g., start) the hearing test; and in accordance with a determination that the current level of environmental noise exceeds (e.g., for a zero or non-zero threshold duration) the threshold noise level, the computer system forgoes display (or, optionally, ceasing to display) of the option to proceed with the hearing test (e.g., and instead displays option 632A as in FIG. 6U1 and/or grayed out option 630Y, as in FIGS. 6U2-6U4). In some embodiments, the computer system detects activation of the option to proceed with the hearing test and, in response, proceeds with (e.g., starts) the hearing test. Providing the user with an option to proceed with the hearing test once the current level of environmental noise drops below the threshold noise level provides the user with visual feedback that the level of environmental noise is sufficiently quiet to proceed with the hearing test, thereby providing improved visual feedback.

In some embodiments, in response to detecting the change in the current level of environmental noise: in accordance with a determination that the current level of environmental noise is below a threshold noise level (e.g., the change in the level of noise causes the noise level to drop below the threshold noise level), the computer system (e.g., 600) displays, via the display generation component (e.g., 602) and concurrently with the visual indication (e.g., 630X) of the current level of environmental noise, text (e.g., 630AB at FIGS. 6V1-6V2 and/or FIGS. 9C-9D) indicating that the current level of environmental noise is sufficiently quiet (e.g., to proceed with the hearing test); and in accordance with a determination that the current level of environmental noise is above the threshold noise level (e.g., the change in the level of noise causes the noise level to rise above the threshold noise level), the computer system (e.g., 600) displays, via the display generation component (e.g., 602), text (e.g., 630AA at FIGS. 6U1-6U4 and/or 9A-9B) indicating that the current level of environmental noise is too loud. Concurrently displaying, with the visual indication, textual information that indicates whether the environmental noise is too loud or sufficiently quiet provides the user with visual feedback about the level of environmental noise, thereby providing improved visual feedback.

In some embodiments, a first audio device (e.g., 620A) of the one or more audio devices (e.g., earphones, headphones, and/or a microphone (of the computer system and/or of a remote device) and/or an audio analyzer) includes one or more microphones and one or more speakers. In some embodiments, the change in the current level of environmental noise is detected using the one or more microphones. In some embodiments, during the hearing test, the computer system (e.g., 600) outputs, via the one or more speakers of the first audio device (e.g., 620A): audio (e.g., a first test tone) with a first volume (e.g., that is independent of a system volume setting) and audio (e.g., a second test tone) with a second volume (e.g., that is independent of the system volume setting) that is different from the first volume. In some embodiments, the computer system uses a microphone of the computer system (e.g., alternative to using the one or more microphones of the first audio device and/or in addition to using the one or more microphones of the first audio device) to detect (e.g., changes in) the current level of environmental noise. In some embodiments, the computer system uses the one or more speakers of the first audio device to output test tones to get user feedback about whether the user has heard the test tones. Using a microphone of the first audio device to detect environmental noise levels and using the speaker of the same first audio device to output test tones during the hearing test enables the computer system to monitor environmental noise and perform the hearing test using the same audio device, thereby improving the man-machine interface.

Note that details of the processes described above with respect to method 1000 (e.g., FIG. 10) are also applicable in an analogous manner to the methods described above. For example, method 1000 optionally includes one or more of the characteristics of the various methods described with reference to methods 700, 800, and 1200. For example, the computer system (e.g., 600) of method 1000 is the same or similar to the computer systems of method 700 and/or method 800. For another example, the audio device (e.g., 620) of method 1000 is the same or similar to the audio devices of methods 700, 800, and 1200. For another example, the hearing test in the different processes is the same hearing test. For brevity, these details are not repeated below.

FIGS. 11A-11N illustrate example user interfaces for performing hearing tests, in accordance with some embodiments. The user interfaces in these figures are used to illustrate the processes described below, including the processes in FIGS. 12A-12B.

FIGS. 11A-11N illustrate example user interfaces for performing a hearing test for one or more ears of a user (e.g., by testing one ear at a time). Computer system 600 provides the user with a user interface that enables the user to adjust the volume of various audio (e.g., that include one or more tones at one or more frequencies) such that the user can identify the faintest/quietest audio that the user can hear for each of the various audio. The user interfaces described with respect to FIGS. 11A-11N can be implemented in conjunction with the user interfaces described with respect to FIGS. 6A-6BH and 9A-9D. In some embodiments, the user interfaces of FIGS. 11A-11N replace one or more of the user interfaces of FIGS. 6A-6BH.

In some embodiments, computer system 600 performs the processes described with respect to FIGS. 6A-6V2, and detects an input (e.g., a tap input and/or a tap-and-hold input 650T at FIG. 6V2) directed to next option 632A at FIG. 6V2 and, in response, displays user interface 11A.

At FIG. 11A, computer system 600 displays user interface 1130, which includes instructions 1130A for providing inputs for the hearing test. In some embodiments, instructions 1130A indicate that during the hearing test the user can provide inputs to change the volume of test audio to adjust to the faintest volume of audio that the user can hear. At FIG. 11A, computer system 600 detects a selection input (e.g., a tap-and-hold input and/or tap input 1150A) directed to next option 1132A and, in response, displays user interface 1110 as shown in FIG. 11B.

At FIG. 11B, user interface 1110 includes instructions 1110A, volume indicators 1140, volume objects 1112A-1112B, next option 1132B, and test details 1110B. Instructions 1110A indicate to the user that the user should adjust the volume of the various test audio to the faintest volume of audio that the user can hear and then proceed with the test. Volume indicators 1140 include a plurality of discrete indicators, with each indicator corresponding to a different audio volume. Volume indicator 1140A corresponds to the lowest volume (e.g., −10 dB, 0 dB, or 5 dB (or dB HL)), volume indicator 1140B corresponds to the highest volume (e.g., 80 dB or 90 dB (or dB HL)), and volume indicator 1140C corresponds to the current volume of the test audio (as indicated by the taller shape and the different color (as compared to volume indicators corresponding to louder volumes) of volume indicator 1140C (e.g., 40 dB or 42 dB). Volume object 1112A, when activated, reduces the volume of the audio (e.g., by one step and/or one level) and volume object 1112B, when activated, increases the volume of the audio (e.g., by one step and/or one level). In some embodiments, each step of volume increase/decrease is determined based on the lowest volume (e.g., the volume corresponding to volume indicator 1140A), the highest volume (e.g., the volume corresponding to volume indicator 1140B), and the number of discrete indicators in volume indicators 1140. For example, when there are 17 discrete volume indicators in volume indicators 1140 and the volume ranges from 0 dB (e.g., lowest volume) to 80 dB (e.g., highest volume), each step corresponds to a change of 5 dB in volume (e.g., increase of 5 dB or decrease of 5 dB). In some embodiments, computer system 600 includes 17 discrete volume indicators, as shown in FIGS. 11B-11K- and 11M. In some embodiments, computer system 600 includes less than or more than 17 discrete volume indicators (e.g., 14, 15, 20, 21, or 25). In some embodiments, each change in volume and/or each discrete volume indicator corresponds to a change (e.g., an increase or decrease) of 1 dB, 2 dB, 2.7 dB, or 3 dB in volume. At FIG. 11B, next option 1132B is optionally not activatable (e.g., as indicated by being grayed out in FIG. 11B) because computer system 600 has not yet received any user inputs requesting to change the volume of first test audio 1134.

At FIG. 11B, computer system 600 is outputting (e.g., via a first speaker of a headphone, headset, and/or earbuds, such as left earbud 620A) first test audio 1134 to a first ear (e.g., a left ear) of the user (e.g., without outputting any audio to a second ear (e.g., via a second speaker or earbud)). In some embodiments, first test audio 1134 includes audio (e.g., a first tone) at a first frequency (e.g., a frequency that is currently being tested), as indicated by test details 1110B. In some embodiments, first test audio 1134 does not include audio (e.g., a second tone, different from the first tone) at a second frequency (e.g., different from the first frequency and/or a frequency that is not currently being tested). In some embodiments, first test audio 1134 includes one or more repeating audio tones (e.g., a tone that pulses repeatedly). In some embodiments, first test audio 1134 is a tone that persists without any breaks in the audio. At FIG. 11B, prior to computer system 600 detecting inputs to change the volume of first test audio 1134, computer system 600 is outputting first test audio 1134 at a volume (e.g., corresponding to volume indicator 1140C) that is independent of the system volume setting (e.g., the computer system starts the hearing test and/or starts the test for first test audio 1134 for the first ear using a volume level that is independent of the system volume of computer system 600). In some embodiments, the volume of first test audio 1134 at FIG. 11B is a volume that is in between the lowest volume and the highest volume (e.g., 1140A corresponds to 0 dB, 1140B corresponds to 80 dB, and 1140C corresponds to 40 dB; computer system 600 outputs first test audio 1134 at 40 dB at FIG. 11B).

At FIG. 11B, while computer system 600 is outputting first test audio 1134 to the first ear of the user via the first speaker, computer system 600 detects an input (e.g., 1150B and/or 1150C) requesting to increase the volume of first test audio 1134 (e.g., because the user cannot hear first test audio 1134). In some embodiments, the input requesting to increase the volume is input 1150B (e.g., a swipe input, a touch input, and/or an air gesture) directed to volume indicators 1140 that includes movement (e.g., away from volume indicator 1140A and toward volume indicator 1140B) and the volume changes based on a magnitude (e.g., distance, speed, and/or acceleration) of the movement of input 1150B (e.g., larger magnitude causes a larger change in volume and smaller magnitude causes a smaller change in volume). In some embodiments, the input requesting to increase the volume is input 1150C (e.g., a tap input, a touch input, and/or an air gesture) directed to volume indicator 1140D and the volume changes based on the location (e.g., being directed to volume indicator 1140D) of input 1150C.

At FIG. 11C, in response to detecting the input (e.g., 1150B and/or 1150C) at FIG. 11B requesting to increase the volume of first test audio 1134, computer system 600 visually updates volume indicators 1140 and increases the volume of first test audio 1134 to a volume corresponding to volume indicator 1140D (e.g., to 75 dB or dB HL) while continuing to output first test audio 1134 to the first ear of the user via the first speaker. Computer system 600 has visually changed (e.g., changed the shape (e.g., increased the height) and/or changed a color of) volume indicator 1140D to indicate that the current volume is at a volume that corresponds to volume indicator 1140D. Volume indicator 1140C has reduced in height to indicate that the current volume does not correspond to volume indicator 1140C. At FIG. 11C, next option 1132B is activatable (e.g., as indicated by not being grayed out). In some embodiments, computer system 600 continuously outputs first test audio 1134 throughout FIGS. 11B-11G.

At FIG. 11C, while outputting first test audio 1134 at the volume corresponding to volume indicator 1140D, computer system 600 detects an input (e.g., 1150D and/or 1150D) requesting to decrease the volume of first test audio 1134 (e.g., because the user can hear first test audio 1134). In some embodiments, the input requesting to decrease the volume is input 1150D (e.g., a swipe input, a touch input, and/or an air gesture) directed to volume indicators 1140 that includes movement (e.g., toward volume indicator 1140A and away from volume indicator 1140B) and the volume changes based on a magnitude (e.g., distance, speed, and/or acceleration) of the movement of input 1150D (e.g., larger magnitude causes a larger change in volume and smaller magnitude causes a smaller change in volume). In some embodiments, the input requesting to decrease the volume is input 1150E (e.g., a tap input, a touch input, and/or an air gesture) directed to volume indicator 1140E and the volume changes based on the location (e.g., being directed to volume indicator 1140E) of input 1150E.

At FIG. 11D, in response to detecting the input (e.g., 1150D and/or 1150E) at FIG. 11C requesting to decrease the volume of first test audio 1134, computer system 600 visually updates volume indicators 1140 and decreases the volume of first test audio 1134 to a volume corresponding to volume indicator 1140E (e.g., to 60 dB or dB HL) while continuing to output first test audio 1134. Computer system 600 has visually changed (e.g., changed the shape (e.g., increased the height) and/or changed a color of) volume indicator 1140E to indicate that the current volume is at a volume that corresponds to volume indicator 1140E. Volume indicator 1140D has reduced in height to indicate that the current volume does not correspond to volume indicator 1140D.

At FIG. 11D, while outputting first test audio 1134 at the volume corresponding to volume indicator 1140E, computer system 600 detects an input (e.g., 1150F) requesting to decrease the volume of first test audio 1134 (e.g., because the user can hear first test audio 1134). In response to detecting the input requesting to decrease the volume, computer system 600 decreases the volume of first test audio 1134 by one step while continuing to output first test audio 1134 to the first ear of the user via the first speaker. In some embodiments, the input requesting to decrease the volume is an input (e.g., a swipe input, a touch input, and/or an air gesture) directed to volume indicators 1140 that includes movement (e.g., toward volume indicator 1140A and away from volume indicator 1140B) and the volume changes based on a magnitude (e.g., distance, speed, and/or acceleration) of the movement of the input (e.g., larger magnitude causes a larger change in volume and smaller magnitude causes a smaller change in volume). In some embodiments, the input requesting to decrease the volume is an input (e.g., a tap input, a touch input, and/or an air gesture) directed to volume indicator 1140F and the volume changes based on the location (e.g., being directed to volume indicator 1140F) of the input. In some embodiments, the input requesting to decrease the volume is input 1150F (e.g., a tap input, a touch input, and/or an air gesture) directed to volume object 1112A and the volume is reduced by one step (e.g., reduced by 5 dB or dB HL), as shown in FIG. 11E.

At FIG. 11E, in response to detecting the input (e.g., 1150F) at FIG. 11D requesting to decrease the volume of first test audio 1134 by one step, computer system 600 visually updates volume indicators 1140 and decreases the volume of first test audio 1134 by one step to a volume corresponding to volume indicator 1140F (e.g., to 55 dB or dB HL) while continuing to output first test audio 1134. Computer system 600 has visually changed (e.g., changed the shape (e.g., increased the height) and/or changed a color of) volume indicator 1140F to indicate that the current volume is at a volume that corresponds to volume indicator 1140F. Volume indicator 1140E has reduced in height to indicate that the current volume does not correspond to volume indicator 1140E.

At FIG. 11E, while outputting first test audio 1134 at the volume corresponding to volume indicator 1140F, computer system 600 detects an input (e.g., 1150G) requesting to decrease the volume of first test audio 1134 (e.g., because the user can hear first test audio 1134). In response to detecting the input requesting to decrease the volume, computer system 600 decreases the volume of first test audio 1134 by one step while continuing to output first test audio 1134 to the first ear of the user via the first speaker. In some embodiments, the input requesting to decrease the volume is an input (e.g., a swipe input, a touch input, and/or an air gesture) directed to volume indicators 1140 that includes movement (e.g., toward volume indicator 1140A and away from volume indicator 1140B) and the volume changes based on a magnitude (e.g., distance, speed, and/or acceleration) of the movement of the input (e.g., larger magnitude causes a larger change in volume and smaller magnitude causes a smaller change in volume). In some embodiments, the input requesting to decrease the volume is an input (e.g., a tap input, a touch input, and/or an air gesture) directed to volume indicator 1140G and the volume changes based on the location (e.g., being directed to volume indicator 1140G) of the input. In some embodiments, the input requesting to decrease the volume is input 1150G (e.g., a tap input, a touch input, and/or an air gesture) directed to volume object 1112A and the volume is reduced by one step (e.g., reduced by 5 dB or dB HL), as shown in FIG. 11F.

At FIG. 11F, in response to detecting the input (e.g., 1150G) at FIG. 11E requesting to decrease the volume of first test audio 1134 by one step, computer system 600 visually updates volume indicators 1140 and decreases the volume of first test audio 1134 by one step to a volume corresponding to volume indicator 1140G (e.g., to 50 dB or dB HL) while continuing to output first test audio 1134. Computer system 600 has visually changed (e.g., changed the shape (e.g., increased the height) and/or changed a color of) volume indicator 1140G to indicate that the current volume is at a volume that corresponds to volume indicator 1140G. Volume indicator 1140F has reduced in height to indicate that the current volume does not correspond to volume indicator 1140F.

At FIG. 11F, while outputting first test audio 1134 at the volume corresponding to volume indicator 1140F, computer system 600 detects an input (e.g., 1150H) requesting to increase the volume of first test audio 1134 (e.g., because the user can no longer hear first test audio 1134). In response to detecting the input requesting to increase the volume, computer system 600 increases the volume of first test audio 1134 by one step while continuing to output first test audio 1134 to the first ear of the user via the first speaker. In some embodiments, the input requesting to increase the volume is an input (e.g., a swipe input, a touch input, and/or an air gesture) directed to volume indicators 1140 that includes movement (e.g., away from volume indicator 1140A and toward volume indicator 1140B) and the volume changes based on a magnitude (e.g., distance, speed, and/or acceleration) of the movement of the input (e.g., larger magnitude causes a larger change in volume and smaller magnitude causes a smaller change in volume). In some embodiments, the input requesting to decrease the volume is an input (e.g., a tap input, a touch input, and/or an air gesture) directed to volume indicator 1140F and the volume changes based on the location (e.g., being directed to volume indicator 1140F) of the input. In some embodiments, the input requesting to decrease the volume is input 1150H (e.g., a tap input, a touch input, and/or an air gesture) directed to volume object 1112B and the volume is increased by one step (e.g., increased by 5 dB or dB HL), as shown in FIG. 11G.

At FIG. 11G, in response to detecting the input (e.g., 1150H) at FIG. 11F requesting to increase the volume of first test audio 1134 by one step, computer system 600 visually updates volume indicators 1140 and increases the volume of first test audio 1134 by one step to a volume corresponding to volume indicator 1140F (e.g., to 55 dB or dB HL) while continuing to output first test audio 1134. Computer system 600 has visually changed (e.g., changed the shape (e.g., increased the height) and/or changed a color of) volume indicator 1140F to indicate that the current volume is at a volume that corresponds to volume indicator 1140F. Volume indicator 1140G has reduced in height to indicate that the current volume does not correspond to volume indicator 1140G. The volume of first test audio 1134 has been increased to a level that the user can again hear. At FIG. 11G, computer system 600 detects user activation (e.g., via tap input 1150I) of next option 1132B, thereby indicating to computer system 600 that the volume corresponding to volume indicator 1140F is the faintest/lowest volume at which the user can hear first test audio 1134 using their first ear via the first speaker.

Accordingly, computer system 600 enables the user to make both course multi-step volume changes (e.g., with tap or swipe inputs directed to volume indicators 1140) and precise single-step volume changes (e.g., with tap or tap-and-hold inputs directed to volume objects 1112A-1112B) to identify the faintest volume at which the user can hear the first test audio. This process also allows the user to focus on the several volumes that are closest to the faintest audible volume for the user, enabling the user to more easily, quickly, and accurately identify the faintest volume at which the user can hear the first test audio to computer system 600.

At FIG. 11H, in response to detecting activation of next option 1132B, computer system 600 ceases to output first test audio 1134 and outputs second test audio 1136 to the first ear of the user via the first speaker. Second test audio 1136 includes audio (e.g., a second tone that is different from the first tone) at a second frequency (e.g., that is different from the first frequency and/or a frequency that is currently being tested), as indicated by test details 1110C. In some embodiments, second test audio 1136 does not include the first tone and/or audio at the first frequency. In some embodiments, second test audio 1136 includes one or more repeating audio tones (e.g., a tone that pulses repeatedly). In some embodiments, second test audio 1136 is a tone that persists without any breaks in the audio. At FIG. 11B, prior to computer system 600 detecting inputs to change the volume of second test audio 1136, computer system 600 is outputting second test audio 1136 at a volume (e.g., corresponding to volume indicator 1140C) that is independent of the system volume setting (e.g., the computer system starts the test for second test audio 1136 for the first ear using a volume level that is independent of the system volume of computer system 600). In some embodiments, the volume of second test audio 1136 at FIG. 11H is a volume that is in between the lowest volume and the highest volume (e.g., 1140A corresponds to 0 dB, 1140B corresponds to 80 dB, and 1140C corresponds to 40 dB; computer system 600 outputs second test audio 1136 at 40 dB at FIG. 11H).

At FIG. 11H, while computer system 600 is outputting second test audio 1136 to the first ear of the user via the first speaker, computer system 600 detects an input (e.g., 1150J, 1150K, and/or 1150L) requesting to decrease the volume of second test audio 1136 (e.g., because the user can hear second test audio 1136). In some embodiments, the input requesting to decrease the volume is input 1150J (e.g., a swipe input, a touch input, and/or an air gesture) directed to volume indicators 1140 that includes movement (e.g., toward volume indicator 1140A and away from volume indicator 1140B) and the volume changes based on a magnitude (e.g., distance, speed, and/or acceleration) of the movement of input 1150J (e.g., larger magnitude causes a larger change in volume and smaller magnitude causes a smaller change in volume). In some embodiments, the input requesting to decrease the volume is input 1150K (e.g., a tap input, a touch input, and/or an air gesture) directed to volume indicator 1140H and the volume changes based on the location (e.g., being directed to volume indicator 1140H) of input 1150K. In some embodiments, the input requesting to decrease the volume is input 1150L (e.g., a tap input, a touch input, and/or an air gesture) directed to volume object 1112A and the volume is reduced by one step (e.g., reduced by 5 dB or dB HL), as shown in FIG. 11I.

At FIG. 11I, in response to detecting the input (e.g., 1150J, 1150K, and/or 1150L) at FIG. 11H requesting to decrease the volume of second test audio 1136, computer system 600 visually updates volume indicators 1140 and decreases the volume of second test audio 11346 to a volume corresponding to volume indicator 1140H (e.g., to 35 dB or dB HL) while continuing to output second test audio 1136 to the first ear of the user via the first speaker. Computer system 600 has visually changed (e.g., changed the shape (e.g., increased the height) and/or changed a color of) volume indicator 1140H to indicate that the current volume is at a volume that corresponds to volume indicator 1140H. Volume indicator 1140C has reduced in height to indicate that the current volume does not correspond to volume indicator 1140C. At FIG. 11I, next option 1132C is activatable (e.g., as indicated by not being grayed out). In some embodiments, computer system 600 continuously outputs second test audio 1136 throughout FIGS. 11H-11K.

At FIG. 11I, while outputting second test audio 1136 at the volume corresponding to volume indicator 1140H, computer system 600 detects an input (e.g., 1150M, 1150N, and/or 1150O) requesting to decrease the volume of second test audio 1136 (e.g., because the user can hear second test audio 1136). In some embodiments, the input requesting to decrease the volume is input 1150M (e.g., a swipe input, a touch input, and/or an air gesture) directed to volume indicators 1140 that includes movement (e.g., toward volume indicator 1140A and away from volume indicator 1140B) and the volume changes based on a magnitude (e.g., distance, speed, and/or acceleration) of the movement of input 1150M (e.g., larger magnitude causes a larger change in volume and smaller magnitude causes a smaller change in volume). In some embodiments, the input requesting to decrease the volume is input 1150N (e.g., a tap input, a touch input, and/or an air gesture) directed to volume indicator 1140I and the volume changes based on the location (e.g., being directed to volume indicator 1140I) of input 1150N. In some embodiments, the input requesting to decrease the volume is input 1150O (e.g., a tap input, a touch input, and/or an air gesture) directed to volume object 1112A and the volume is reduced by one step (e.g., reduced by 5 dB or dB HL), as shown in FIG. 11J.

At FIG. 11J, in response to detecting the input (e.g., 1150M, 1150N, and/or 1150O) at FIG. 11I requesting to decrease the volume of second test audio 1136, computer system 600 visually updates volume indicators 1140 and decreases the volume of second test audio 1136 to a volume corresponding to volume indicator 1140I (e.g., to 30 dB or dB HL) while continuing to output second test audio 1136. Computer system 600 has visually changed (e.g., changed the shape (e.g., increased the height) and/or changed a color of) volume indicator 1140I to indicate that the current volume is at a volume that corresponds to volume indicator 1140I. Volume indicator 1140H has reduced in height to indicate that the current volume does not correspond to volume indicator 1140H.

At FIG. 11J, while outputting second test audio 1136 at the volume corresponding to volume indicator 1140I, computer system 600 detects an input (e.g., 1150P, 1150Q, and/or 1150R) requesting to increase the volume of second test audio 1136 (e.g., because the user cannot hear second test audio 1136). In response to detecting the input requesting to increase the volume, computer system 600 increases the volume of second test audio 1136 by one step while continuing to output second test audio 1136 to the first ear of the user via the first speaker. In some embodiments, the input requesting to increase the volume is input 1150P (e.g., a swipe input, a touch input, and/or an air gesture) directed to volume indicators 1140 that includes movement (e.g., away from volume indicator 1140A and towards volume indicator 1140B) and the volume changes based on a magnitude (e.g., distance, speed, and/or acceleration) of the movement of input 1150P (e.g., larger magnitude causes a larger change in volume and smaller magnitude causes a smaller change in volume). In some embodiments, the input requesting to increase the volume is input 1150Q (e.g., a tap input, a touch input, and/or an air gesture) directed to volume indicator 1140H and the volume changes based on the location (e.g., being directed to volume indicator 1140H) of the input. In some embodiments, the input requesting to increase the volume is input 1150R (e.g., a tap input, a touch input, and/or an air gesture) directed to volume object 1112B and the volume is increased by one step (e.g., increased by 5 dB or dB HL), as shown in FIG. 11K.

At FIG. 11K, in response to detecting the input (e.g., 1150P, 1150Q, and/or 1150R) at FIG. 11J requesting to increase the volume of second test audio 1136, computer system 600 visually updates volume indicators 1140 and increases the volume of second test audio 1136 to a volume corresponding to volume indicator 1140H (e.g., to 35 dB or dB HL) while continuing to output second test audio 1136. Computer system 600 has visually changed (e.g., changed the shape (e.g., increased the height) and/or changed a color of) volume indicator 1140H to indicate that the current volume is at a volume that corresponds to volume indicator 1140H. Volume indicator 1140I has reduced in height to indicate that the current volume does not correspond to volume indicator 1140I. At FIG. 11K, computer system 600 detects user activation (e.g., via tap input 1150S) of next option 1132C, thereby indicating to computer system 600 that the volume corresponding to volume indicator 1140H is the faintest/lowest volume at which the user can hear second test audio 1136 using their first ear via the first speaker.

Accordingly, computer system 600 enables the user to make both course multi-step volume changes (e.g., with tap or swipe inputs directed to volume indicators 1140) and precise single-step volume changes (e.g., with tap or tap-and-hold inputs directed to volume objects 1112A-1112B) to identify the faintest volume at which the user can hear the second test audio. This process also allows the user to focus on the several volumes that are closest to the faintest audible volume for the user, enabling the user to more easily, quickly, and accurately identify the faintest volume at which the user can hear the second test audio to computer system 600.

In some embodiments, in response to detecting user activation (e.g., via tap input 1150S) of next option 1132C, the technique continues to test the user's first ear using the first speaker using one or more additional test audio and having the user adjust the volume to the faintest volume at which the user can hear each respective test audio. In some embodiments, computer system 600 tests the user's first ear using the first speaker with 6, 8, or 10 different test audio (e.g., with each test audio including a different frequency of audio). In the example of FIGS. 11B-11K, computer system 600 tests for a total of 6 different test audio, as indicated by test details 1110B and 1110C.

At FIG. 11L, after completing testing of the 6, 8, or 10 test audio for the user's first ear using the first speaker, computer system 600 displays user interface 1114, which indicates that testing of the first ear is complete and that testing of the user's second ear (e.g., using a second speaker that is optionally different from the first speaker) will begin next. In some embodiments, computer system 600 also outputs spoken audio 1138A indicating that testing of the first ear is complete and that testing of the user's second ear will begin next. At FIG. 11L, computer system 600 detects a selection input (e.g., a tap-and-hold input and/or tap input 1150T) directed to next option 1132D and, in response, displays user interface 1116 as shown in FIG. 11M.

At FIG. 11M, computer system 600 begins testing the user's second ear (e.g., the user's right ear) using a second speaker (e.g., 620B). At FIG. 11M, user interface 1116 includes instructions 1116A, volume indicators 1140, volume objects 1112A-1112B, next option 1132E, and test details 1116B. Instructions 1116A indicate to the user that the user should adjust the volume of the various test audio to the faintest volume of audio that the user can hear and then proceed with the test. Volume indicators 1140 operate the same as described above and include a plurality of discrete indicators, with each indicator corresponding to a different audio volume. Volume indicator 1140A corresponds to the lowest volume (e.g., −10 dB, 0 dB, or 5 dB (or dB HL)), volume indicator 1140B corresponds to the highest volume (e.g., 80 dB or 90 dB (or dB HL)), and volume indicator 1140C corresponds to the current volume of the test audio (as indicated by the taller shape and the different color (as compared to volume indicators corresponding to louder volumes) of volume indicator 1140C (e.g., 40 dB or 42 dB). Volume object 1112A, when activated, reduces the volume of the audio (e.g., by one step and/or one level) and volume object 1112B, when activated, increases the volume of the audio (e.g., by one step and/or one level). Similar to the technique described above, the user provides inputs to change the volume of first test audio 1134 being output to the user's second ear using the second speaker to identify the faintest/quietest audio that the user can hear. Computer system 600 detects the user's inputs, changes the volumes, and then logs the faintest volume that the user can hear. Once computer system 600 has tested the user's second ear using the second speaker with the 6, 8, or 10 different test audio (e.g., with each test audio including a different frequency of audio) and identified the faintest volume that the user can hear for each test audio, computer system 600 displays user interface 1118 and optionally outputs spoken audio 1138B.

At FIG. 11N, computer system 600 detects user activation (e.g., via tap input 1150U) of show results option 1132F and, in response, initiates a process to show the results of the testing, such as an audiogram. In some embodiments, in response to detecting user activation of show results option 1132F, computer system 600 displays hearing test results, as shown in user interface 660 at FIG. 6AI. In some embodiments, computer system 600 further proceeds to receive additional user inputs to navigate among the user interfaces of FIGS. 6AI-6BH. In some embodiments, during the hearing testing described with respect to FIGS. 11B-11M, computer system 600 determines that the environment is too loud for continuing the hearing test and pauses (or ends) the hearing test, as shown in and described with respect to FIGS. 6AA1-6AA4.

FIGS. 12A-12B are a flow diagram illustrating methods of performing hearing tests, in accordance with some embodiments. Method 1200 is performed at a computer system (e.g., 100, 300, 500, and/or 600) (e.g., a smart phone, a smart watch, a tablet, a laptop, a desktop, a wearable device, wrist-worn device, and/or head-mounted device) that is in communication with one or more display generation components (e.g., 602) (e.g., one or more displays, touch-sensitive displays, display controllers, head-mounted displays, and/or projectors), one or more input devices (e.g., 602) (e.g., one or more touch-sensitive surfaces, touch-sensitive displays, buttons, rotatable input mechanisms, depressible and rotatable input mechanisms, cameras, and/or accelerometers), and one or more audio devices (e.g., 620A-620B) (e.g., one or more earphones, headphones, and/or speakers). Some operations in method 1200 are, optionally, combined, the orders of some operations are, optionally, changed, and some operations are, optionally, omitted.

As described below, method 1200 provides an intuitive way for performing hearing tests. The method reduces the cognitive burden on a user for conducting the hearing tests, thereby creating a more efficient human-machine interface. For battery-operated computing devices, enabling a user to conduct hearing tests faster and more efficiently conserves power and increases the time between battery charges.

During a process (1202) to perform (e.g., using the computer system) a hearing test (e.g., during the hearing test and/or during a process that includes outputting audio as part of an audiometry test), displaying (1204), via the one or more display generation components (e.g., 602), a user interface (e.g., 1110) that includes a plurality of discrete volume indicators (e.g., 1140) and a visual indication (e.g., 1140C at FIG. 11B and/or 1140D at FIG. 11C) of a current volume for outputting audio (e.g., 1134 and/or 1136) (e.g., test tones and/or audio for testing a user's hearing) during the hearing test, wherein the plurality of discrete volume indicators include: a volume indicator (1206) (e.g., 1140A) that corresponds to a minimum volume for outputting audio (e.g., test tones and/or audio for testing a user's hearing) during the hearing test, a volume indicator (1208) (e.g., 1140B) that corresponds to a maximum volume for outputting audio (e.g., test tones and/or audio for testing a user's hearing) during the hearing test, and a volume indicator (1210) (e.g., 1140C) that corresponds to the current volume (e.g., the first volume) for outputting audio (e.g., test tones and/or audio for testing a user's hearing) during the hearing test.

During the process (1202) to perform (e.g., using the computer system) the hearing test, while displaying the user interface (e.g., 1110) that includes the plurality of discrete volume indicators (e.g., 1140) and the visual indication of a current volume for outputting audio during the hearing test, outputting (1212), via a first speaker (e.g., 620A and/or 620B) (e.g., a left speaker, a speaker corresponding to a left ear of the user, a right speaker, and/or a speaker corresponding to a right ear of the user) of the one or more audio devices, a first audio (e.g., 1134) (e.g., a first test tone and/or a first test audio) with a first volume (e.g., 1134 at FIGS. 11B and/or 11D) (e.g., that is independent of a system volume setting and/or that is the same as the current volume for outputting audio during the hearing test).

During the process (1202) to perform (e.g., using the computer system) the hearing test, while outputting, via the first speaker (e.g., 620A and/or 620B) of the one or more audio devices, the first audio (e.g., at the first volume), detecting (1214), via the one or more input devices, a user input (e.g., 1150B, 1150C, 1150F, and/or 1150H) (e.g., a swipe input, a tap input, a touch input, and/or an air gesture) directed to the user interface (e.g., 1110) (e.g., directed to the plurality of discrete volume indicators and/or to a different portion of the user interface).

During the process (1202) to perform (e.g., using the computer system) the hearing test, in response (1216) to detecting the user input (e.g., 1150B, 1150C, 1150F, and/or 1150H) directed to the user interface (and, optionally, while continuing to output the first audio): changing (1218) (e.g., increasing and/or decreasing), based on the user input (e.g., the amount and/or magnitude of the change in volume of the first audio is based on (e.g., a magnitude of and/or a location of) the user input), a volume of the first audio (e.g., 1134) from the first volume to a second volume (e.g., 1134 at FIGS. 11C and/or 11E) that is different from the first volume and updating (1220), via the one or more display generation components (e.g., 602), the user interface (e.g., 1140 of 1110) (e.g., the plurality of discrete volume indicators and/or the visual indication of the current volume) to indicate that the current volume for outputting audio during the hearing test is the second volume. Changing the volume of the first audio and updating the user interface to indicate the current volume provides the user with visual feedback about the current volume at which the first audio is being output, thereby providing improved visual feedback, and enables the user to adjust the current volume to various levels while visualizing the various volume levels (e.g., via the indication of the current volume and/or plurality of discrete volume indicators include).

In some embodiments, the computer system (e.g., 600) continuously outputs the first audio (e.g., 1134) (e.g., a first test tone and/or a first test audio) while changing the volume of the first audio from the first volume to the second volume. In some embodiments, the computer system changes the volume of the first audio in response to the user inputs while the computer system continuously outputs the first audio via one or more speakers. Continuously outputting the first audio while the volume changes provide the user with audio feedback about the change in volume of the first audio and enables the user to concentrate and listen for the first audio, knowing that the first audio is being output, thereby improving the man-machine interface.

In some embodiments, the first audio (e.g., 1134) includes audio at a first frequency (e.g., 250 Hz, 500 Hz, 1 kHz, 2 kHz, 4 kHz, or 8 kHz) that is continuously output while changing the volume of the first audio from the first volume to the second volume. In some embodiments, the first audio includes only audio at the first frequency. In some embodiments, the computer system changes the volume of the first audio while maintaining the frequency (ies) of the audio while the computer system continuously outputs the first audio via one or more speakers. In some embodiments, first audio includes audio at a plurality of frequencies (e.g., 250 Hz, 500 Hz, 1 kHz, 2 kHz, 4 kHz, and/or 8 kHz) that are continuously output while changing the volume of the first audio from the first volume to the second volume. Continuously outputting audio at the first frequency while the volume changes provide the user with audio feedback about the change in volume of the audio and enables the user to concentrate and listen for the audio at the first frequency, knowing that the audio is being output, thereby improving the man-machine interface.

In some embodiments, detecting the user input (e.g., 1150B and/or 1150D) directed to the user interface includes detecting directional movement (e.g., via a mouse pointer, via a touch-sensitive surface, and/or via an air gesture) of the user input (e.g., in relation to the user interface) (e.g., a swipe input and/or a drag input). In some embodiments, the change in the volume is based on the direction of the directional movement of the user input. In some embodiments, the amount of volume change is based on a magnitude (e.g., distance, speed, and/or acceleration) of the movement of the user input. In some embodiments, left and right movements of the input correspond to changing the volume. In some embodiments, up and down movements of the input correspond to changing the volume. In some embodiments, the movement of the input directed to the user interface is in a direction that is toward the volume indicator that corresponds to a minimum volume for outputting audio during the hearing test or toward the volume indicator that corresponds to a maximum volume for outputting audio during the hearing test. In some embodiments, the user input directed to the user interface includes movement across one or more discrete volume indicators of the plurality of discrete volume indicators. Changing the volume of the first audio based on user input that includes movement enables the user to more quickly and easily change the volume of the first audio among the various available volumes to identify the faintest volume at which the user can hear the first audio, thereby providing improved audio feedback and improving the man-machine interface.

In some embodiments, changing, based on the user input, the volume of the first audio from the first volume to the second volume includes change the volume in discrete (e.g., distinct) steps (e.g., in a single step) from the first volume to the second volume (e.g., without changing to a volume that is between the first volume and the second volume). In some embodiments, the volume change from the first volume to the second volume is not a continuous volume change that happens smoothly and/or gradually. In some embodiments, the volume remains at the first volume during a first portion of movement of the user input, changes to the second volume during a second portion of movement of the user input, and remains at the second volume during a third portion of the movement of the user input. In some embodiments, the changes in volume of the first audio are discrete changes, rather than continuous changes. Changing the volume of the first audio in discrete steps makes it easier for the user to perceive that the volume has changed, thereby providing improved audio feedback. The discrete changes in volume also enables the user to more easily identify a faintest volume at which the user can hear the first audio, thereby improving the man-machine interface.

In some embodiments, the user input (e.g., a tap input and/or a pinch air gesture) is directed to a respective user interface object (e.g., 1140E at FIG. 11C, 1112A at FIG. 11D, and/or 1112B at FIG. 11E) of the user interface (e.g., 1110) (e.g., is directed to a respective volume indicator of the plurality of discrete volume indicators and the second volume corresponds to the respective volume indicator or is directed to a volume up or down user interface object). In some embodiments, the change in volume is not based on a magnitude of the user input directed to the respective user interface object. In some embodiments, the change in volume is based on a location of the user input (e.g., a location of a touch on a touch-sensitive surface and/or a location of a user's gaze when a pinch gesture is detected). In some embodiments, the user input is directed to an increase volume user interface object or a decrease volume user interface object and, in accordance with a determination that the user input is directed to the increase volume user interface object, the volume increases from the first volume to the second volume and in accordance with a determination that the user input is directed to the decrease volume user interface object, the volume decreases from the first volume to the second volume.

In some embodiments, the computer system (e.g., 600) displays (e.g., via the one or more display generation components and during the hearing test), as part of the user interface and concurrently with the plurality of discrete volume indicators (e.g., 1140) and the visual indication of the current volume (e.g., increased height of 1140E at FIG. 11D), an increase volume user interface object (e.g., 1112A) and a decrease volume user interface object (e.g., 1112B). In some embodiments, changing, based on the user input, the volume of the first audio from the first volume to the second volume includes: in accordance with a determination that the user input is directed to the increase volume user interface object (e.g., a tap input and/or a pinch air gesture), increasing the volume from the first volume to the second volume (e.g., as in the transition from FIG. 11F to FIG. 11G); and in accordance with a determination that the user input is directed to the decrease volume user interface object (e.g., a tap input and/or a pinch air gesture), decreasing the volume from the first volume to the second volume (e.g., as in the transition from FIG. 11E to 11F). In some embodiments, changing, based on the user input, the volume of the first audio from the first volume to the second volume includes: in accordance with a determination that the user input is directed to the plurality of discrete volume indicators (e.g., a tap input, a swipe input, and/or a drag input), changing the volume based on a location and/or distance of the user input. Providing the user with ability to change the volume discretely by selecting volume up and/or volume down buttons in the user interface enables the user to more accurately identify the faintest volume at which the first audio can be heard, thereby improving the man-machine interface.

In some embodiments, changing (e.g., increasing and/or decreasing), based on the user input (e.g., the amount and/or magnitude of the change in volume of the first audio is based on (e.g., a magnitude of and/or a location of) the user input), the volume of the first audio from the first volume to the second volume includes making a plurality of discrete volume changes (e.g., increase and/or decrease in dB). In some embodiments, a magnitude of a first discrete volume change of the plurality of discrete volume changes is based on the minimum volume for outputting audio during the hearing test, the maximum volume for outputting audio during the hearing test, and the number of (e.g., user-accessible, supported, and/or available) discrete volume levels between the minimum volume and the maximum volume. In some embodiments, each discrete change in volume (e.g., increase/decrease in dB) is based on (e.g., has a magnitude equal to): (dB of the maximum volume-dB of the minimum volume)/number of discrete volume levels between the minimum volume and the maximum volume. In some embodiments, the number of discrete volume levels between the minimum volume and the maximum volume corresponds to (e.g., equals) the number of discrete volume indicators in the plurality of discrete volume indicators. In some embodiments, the number of discrete volume levels and the number of discrete volume indicators (e.g., bars and/or objects) is 25, 28, or 31. In some embodiments, the discrete change in volume is 2 dB, 2.7 dB, or 3 dB (e.g., each bar corresponds to 2 dB, 2.7 dB, or 3 dB). Basing the discrete levels of volume change (e.g., in dB) on the minimum volume for outputting audio during the hearing test, the maximum volume for outputting audio during the hearing test, and the number of discrete volume levels between the minimum volume and the maximum volume enables the computer system to provide consistent volume changes for each discrete volume change, thereby enabling the user to more accurately identify the faintest volume at which the first audio can be heard and improving the man-machine interface.

In some embodiments, each discrete change in volume level between the minimum volume and the maximum volume is by a same amount (e.g., in dB). In some embodiments, each discrete volume indicator in the plurality of discrete volume indicators corresponds to an equal change in volume. Changing the volume using equal discrete changes enables the user to more accurately identify the faintest volume at which the first audio can be heard and improves the man-machine interface.

In some embodiments, displaying the visual indication of the current volume for outputting audio (e.g., test tones and/or audio for testing a user's hearing) during the hearing test includes visually differentiating (e.g., having a different color, a different height, a different width, and/or a different shape) a respective discrete volume indicator (e.g., 1140F at FIG. 11G), of the plurality of discrete volume indicators (e.g., 1140), that corresponds to the current volume from respective discrete volume indicators (e.g., 1140G and/or 1140E), of the plurality of discrete volume indicators (e.g., 1140), that do not correspond to the current volume. Visually differentiating a particular discrete volume indicator (in comparison to the other discrete volume indicators) to indicate the current volume provides the user with visual feedback about the volume and how many discrete volume steps the volume can be increased and/or decreased, thereby providing improved visual feedback.

In some embodiments, the computer system (e.g., 600) displays (e.g., via the one or more display generation components and during the hearing test), as part of the user interface and concurrently with the plurality of discrete volume indicators and the visual indication of the current volume, a confirm user interface option (e.g., 1132B, 1132C, and/or 1132E). While outputting, via the first speaker, the first audio, the computer system (e.g., 600) detects, via the one or more input devices, an input (e.g., 1150I and/or 1150S) (e.g., a tap input and/or a pinch air gesture) directed to the confirm user interface option (e.g., 1132B, 1132C, and/or 1132E). In response to detecting the input (e.g., 1150I and/or 1150S) directed to the confirm user interface option: in accordance with a determination that the current volume is a first respective volume, the computer system (e.g., 600) identifies the first respective volume as the faintest (e.g., lowest and/or quietest) volume at which a user can hear (e.g., using a first ear that corresponds to the first speaker) the first audio (e.g., the first test tone and/or the first test audio) and in accordance with a determination that the current volume is a second respective volume that is different from the first respective volume, the computer system (e.g., 600) identifies the second respective volume as the faintest (e.g., lowest and/or quietest) volume (e.g., different from the first volume and/or the second volume) at which the user can hear (e.g., using the first ear that corresponds to the first speaker) the first audio (e.g., the first test tone and/or the first test audio). In some embodiments, while the first audio is being output by the first speaker (e.g., to a first ear of the user), the computer system receives one or more user inputs that change the volume of the first audio. Once the user identifies the faintest volume at which the user can hear the first audio and the current volume is set to that faintest volume, the user selects the confirm user interface option and the computer system stores an indication that the current volume is the faintest volume at which the user can hear the first audio using their first ear. Identifying the current volume as the faintest volume at which the user can hear the first audio when the user provides input directed to the confirm user interface option allows the user to repeatedly change the volume of the first audio (e.g., both up and down) to make sure the user has properly selected faintest volume at which the user can hear the first audio, thereby making the test more accurate.

In some embodiments, subsequent to detecting the input (e.g., 1150I at FIG. 11G) (e.g., a tap input and/or a pinch air gesture) directed to the confirm user interface option while outputting, via the first speaker, the first audio: the computer system (e.g., 600) displays, via the one or more display generation components, the plurality of discrete volume indicators (e.g., 1140 at FIG. 11H) and concurrently outputting, via the first speaker (e.g., a left speaker, a speaker corresponding to a left ear of the user, a right speaker, and/or a speaker corresponding to a right ear of the user) of the one or more audio devices, a second audio (e.g., 1136 at FIG. 11H) (e.g., a second test tone and/or a second test audio), different from the first audio (e.g., with a volume (e.g., with the first volume) that is independent of the second respective volume identified as the faintest volume at which the user can hear the first audio and/or with a volume that is independent of a system volume setting). In some embodiments, once user confirms the faintest volume at which the user can hear the first tone with their first ear, the computer system transitions to testing to identify the faintest volume at which the user can hear the second tone with their first ear. Subsequently, the computer system transitions to testing to identify the faintest volumes at which the user can hear the first tone and the second tone with their second ear (that is different from their first ear) (e.g., using a second speaker that is different from the first speaker). In some embodiments, the computer system performs testing on 4, 6, 8, or 10 different audios for the first ear before proceeding to perform testing on the same 4, 6, 8, or 10 different audios for the second ear. Concurrently displaying the plurality of volume indicators and outputting a second audio enables the computer system to test the user's hearing for a different frequency, thereby enabling the computer system to more accurately determine the user's hearing sensitivity at different frequencies and improves the accuracy of the test.

Note that details of the processes described above with respect to method 1200 (e.g., FIGS. 12A-12B) are also applicable in an analogous manner to the methods described above. For example, method 1200 optionally includes one or more of the characteristics of the various methods described with reference to methods 700, 800, and 1100. For example, the computer system (e.g., 600) of method 1200 is the same or similar to the computer systems of methods 700, 800, and 1100. For another example, the audio device (e.g., 620) of method 1000 is the same or similar to the audio devices of methods 700, 800, 1200. For another example, the hearing test in the different processes is the same hearing test. For brevity, these details are not repeated below.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying drawings, it is to be noted that various changes and modifications will become apparent to those skilled in the art. Such changes and modifications are to be understood as being included within the scope of the disclosure and examples as defined by the claims.

As described above, one aspect of the present technology is the gathering and use of data available from various sources to improve the delivery to users of audio content. The present disclosure contemplates that in some instances, this gathered data may include personal information data that uniquely identifies or can be used to contact or locate a specific person. Such personal information data can include demographic data, location-based data, telephone numbers, email addresses, social network IDs, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to provide clearer audio. Accordingly, use of such personal information data enables users to have better hearing. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, hearing aid services, the present technology can be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, hearing preferences can be selected by inferring preferences based on non-personal information data or a bare minimum amount of personal information, such as the content being requested by the device associated with a user, other non-personal information available to the system, or publicly available information.

What is claimed is:

1. A computer system configured to communicate with one or more input devices, comprising:
one or more processors; and
memory storing one or more programs configured to be executed by the one or more processors, the one or more programs including instructions for:
while not paired with a first audio device, detecting, via the one or more input devices, a request to pair the first audio device with the computer system; and
in response to detecting the request to pair the first audio device with the computer system, initiating a process to pair the first audio device with the computer system, wherein the process to pair the first audio device with the computer system includes:
pairing the first audio device with the computer system;
in accordance with a determination that the first audio device is a first type of audio device, providing an option to perform a hearing test that uses the first audio device; and
in accordance with a determination that the first audio device is a second type of audio device that is different from the first type of audio device, forgoing providing the option to perform the hearing test that uses the first audio device.

2. The computer system of claim 1, wherein:
the process to pair the first audio device with the computer system includes:
in accordance with a determination that the first audio device is a first type of audio device, providing an option to not perform the hearing test; and
the one or more programs further including instructions for:
detecting, via the one or more input devices, an option selection user input; and
in response to detecting the option selection user input:
in accordance with a determination that the option selection user input is directed to the option to perform the hearing test, proceeding with a process to perform the hearing test; and
in accordance with a determination that the option selection user input is directed to the option to not perform the hearing test, forgoing proceeding with the process to perform the hearing test.

3. The computer system of claim 1, the one or more programs further including instructions for:
generating, based on performance of the hearing test, audiogram data.

4. The computer system of claim 1, the one or more programs further including instructions for:
subsequent to pairing the first audio device with the computer system, performing the hearing test using the first audio device.

5. The computer system of claim 1, wherein the computer system is configured to communicate with one or more display generation components, the one or more programs further including instructions for:
subsequent to performing the process to pair the first audio device with the computer system, displaying, via the one or more display generation components, a settings user interface that includes an option to perform a hearing test using the first audio device;
detecting, via the one or more input devices, an input directed to the option to perform the hearing test using the first audio device; and
in response to detecting the input directed to the option to perform the hearing test using the first audio device, proceeding with a process to perform the hearing test.

6. The computer system of claim 1, wherein the first audio device includes one or more speakers and one or more microphones, the one or more programs further including instructions for:
prior to perform the hearing test:
initiating a process to perform an audio input and audio output assessment of the first audio device using the one or more speakers and the one or more microphones of the first audio device; and
after performing the audio input and audio output assessment of the first audio device:
in accordance with a determination that the first audio device passed the audio input and audio output assessment, proceeding with a process to perform the hearing test that uses the first audio device; and
in accordance with a determination that the first audio device did not pass the audio input and audio output assessment, forgoing proceeding with the process to perform the hearing test.

7. The computer system of claim 1, wherein the first audio device includes one or more microphones, the one or more instructions further including instructions for:
during the hearing test, detecting, via the one or more microphones of the first audio device, a level of environmental noise; and
in response to detecting the level of environmental noise:
in accordance with a determination that the level of environmental noise is more than a threshold level, interrupting the hearing test; and
in accordance with a determination that the level of environmental noise is less than the threshold level, forgoing interrupting the hearing test.

8. The computer system of claim 7, the one or more programs further including instructions for:
in response to detecting the level of environmental noise:
in accordance with the determination that the level of environmental noise is more than the threshold level, providing an option to continue the hearing test.

9. The computer system of claim 1, wherein the first audio device includes one or more speakers and one or more microphones, the one or more programs further including instructions for:
subsequent to completing the hearing test, outputting, via the one or more speakers of the first audio device, first audio using a volume setting that is based on a user-selected volume.

10. The computer system of claim 1, wherein the first audio device includes one or more speakers, the one or more programs further including instructions for:
during the hearing test:
detecting, via the one or more input devices, a volume change user input;
in response to detecting the volume change user input, changing, based on the volume change user input, a volume setting from a first value to a second value that is different from the first value; and
while the volume setting is at the second value, outputting, via the one or more speakers, audio that is not based on the second value of the volume setting.

11. The computer system of claim 1, the one or more programs further including instructions for:
changing a volume setting from a user-selected volume to a system-selected volume; and
during the hearing test, maintaining the volume setting at the system-selected volume.

12. The computer system of claim 11, wherein the first audio device includes one or more speakers, the one or more programs further including instructions for:
during the hearing test, outputting, via the one or more speakers of the first audio device:
audio with a first volume that is independent of a system volume setting; and
audio with a second volume, different from the first volume, that is independent of the system volume setting.

13. The computer system of claim 1, wherein the first audio device includes one or more speakers and one or more microphones, the one or more programs further including instructions for:
performing, by outputting test audio via the one or more speakers of the first audio device, the hearing test to generate a set of audio characteristics using audiogram data;
receiving, via the one or more microphones of the first audio device, audio; and
outputting, via the one or more speakers of the first audio device, modified audio of the received audio, wherein the modified audio has been adjusted based on the set of audio characteristics.

14. The computer system of claim 1, wherein the first audio device includes one or more speakers, the one or more programs further including instructions for:
during the hearing test:
outputting, via the one or more speakers of the first audio device, first audio; and
subsequent to outputting the first audio, monitoring for user input indicating that a user of the computer system heard the first audio.

15. A non-transitory computer-readable storage medium storing one or more programs configured to be executed by one or more processors of a computer system that is in communication with one or more input devices, the one or more programs including instructions for:
while not paired with a first audio device, detecting, via the one or more input devices, a request to pair the first audio device with the computer system; and
in response to detecting the request to pair the first audio device with the computer system, initiating a process to pair the first audio device with the computer system, wherein the process to pair the first audio device with the computer system includes:
pairing the first audio device with the computer system;
in accordance with a determination that the first audio device is a first type of audio device, providing an option to perform a hearing test that uses the first audio device; and
in accordance with a determination that the first audio device is a second type of audio device that is different from the first type of audio device, forgoing providing the option to perform the hearing test that uses the first audio device.

16. A method, comprising:
at a computer system that is in communication with one or more input devices:
while not paired with a first audio device, detecting, via the one or more input devices, a request to pair the first audio device with the computer system; and
in response to detecting the request to pair the first audio device with the computer system, initiating a process to pair the first audio device with the computer system, wherein the process to pair the first audio device with the computer system includes:
pairing the first audio device with the computer system;
in accordance with a determination that the first audio device is a first type of audio device, providing an option to perform a hearing test that uses the first audio device; and
in accordance with a determination that the first audio device is a second type of audio device that is different from the first type of audio device, forgoing providing the option to perform the hearing test that uses the first audio device.
